(19) United States
(12) Patent Application Publication
(10) Pub. No.: US 2012/0028879 A1
(43) Pub. Date: Feb. 2, 2012

(54) HIGHLY SENSITIVE DETECTION METHOD FOR HIGHLY VIRULENT ORAL CAVITY BACTERIA

(75) Inventors: Kazuo Umemura, Hamamatsu-shi (JP); Kazuya Hokamura, Hamamatsu-shi (JP); Kazuhiko Nakano, Osaka (JP); Takashi Ooshima, Osaka (JP); Ryota Nomura, Osaka (JP); Koichiro Wada, Osaka (JP)

(73) Assignee: NATIONAL UNIVERSITY CORPORATION HAMAMATSU UNIVERSITY SCHOOL OF MEDICINE, SHIZUOKA (JP)

(21) Appl. No.: 13/260,840

(22) PCT Filed: Mar. 15, 2010
(86) PCT No.: PCT/JP2010/054364

§ 371 Date: Sep. 28, 2011

(30) Foreign Application Priority Data
Mar. 31, 2009 (JP) 2009-088239

(51) Int. Cl. A61K 38/00; C12Q 1/04; C07K 14/00; G01N 33/554

(52) U.S. Cl. 514/1.1; 435/7.2; 435/34; 530/350

(57) ABSTRACT

Provided is a method that involves the detection of protein antigen (PA) and/or collagen-binding protein (CBP) of oral cavity bacteria in a sample, and in which oral cavity bacteria that exacerbate hemolysis are detected for and/or subjects at high-risk for hemolysis aggravation are screened anchor the level of risk of the hemolysis aggravation in a subject is assessed if a PA is not detected anchor a CBP is detected in the sample. Also provided are a detection reagent and kit for use in the method.

### TECHNICAL FIELD

The present invention is directed to a method of detecting an oral bacterium that causes hemorrhage aggravation, a method of screening for a subject at a high risk of hemorrhage aggravation, a method of determining the risk of hemorrhage aggravation in a subject, as well as detection reagents and kits for the use in these methods.

### BACKGROUND ARTS

Conditions which involve hemorrhage through vascular injuries include such as hemorrhage by a rupture of a blood vessel that caused by a traumatic injury or pressure, hemorrhage at delivery and intracerebral hemorrhage. In a case of intracerebral hemorrhage, for instance, a severe disorder may be brought about by an injury of the neuronal tissue due to compression or necrosis of the brain associated with hemorrhage, or by neurologic symptoms due to a vascular spasm in cerebrum induced by bleeding in a case of subarachnoid hemorrhage etc. In order to improve the prognosis of hemorrhage, an effective treatment of hemorrhage (hemostasis) as well as the prevention of hemorrhage aggravation is necessary, and diagnosis of the risk of hemorrhage aggravation is important.

Markers used in the diagnosis of a disease which involves hemorrhage include, for example, Apo C-III, serum amyloid A, Apo C-I, antithrombin III fragment and Apo A-I (Patent literature 1) for the diagnoses of the possibility of a stroke, cerebrospinal fluid markers of cerebral ischemia such as adenylate kinase as well as β-thromboglobulin, vascular cell adhesion molecule (VCAM) and atriuretic peptide for the diagnoses of the prognosis of a stroke and cerebral injury, and von Willebrand factor (vWF), vascular endothelial growth factor (VEGF) and matrix-metalloprotease-9 (MMP-9) for the prediction of cerebral vascular spasm which occurs later (Patent literature 2). However, these are all markers for detecting already-happening bleeding in vivo, and cannot diagnose the risk of hemorrhage aggravation.

Accordingly, there have been needs for the establishment of a method of determining or screening a risk of causing aggravation of hemorrhage or a subject with such a risk, and a method of preventing or treating.

### PRIOR ART LITERATURES

### Patent Literatures

[Patent literature 1] JP A No. 2007-502401
[Patent literature 2] JP A No. 2005-522669

### SUMMARY OF INVENTION

### Problems to be Solved by the Invention

Accordingly, an object of the invention is to identify the responsible factor that causes aggravation of hemorrhage, and to construct a system for rapidly and readily specifying a patient having a risk of hemorrhage aggravation. Another object of the invention is to prevent the aggravation of hemorrhage in an individual having such a risk.

### Means for Solving the Problems

The inventors carried out an intensive study to achieve the aforementioned objects and found that hemorrhage is aggravated in a subject who has been infected with a particular strain of S. mutans. By additional studies the inventors found that the most severe virulence is exerted by bacterial strains that do not carry a protein antigen PA (Protein Antigen, also known as PAc, SpaP, antigen I/II, antigen B, SR, IF, P1, MSL-1), i.e., a major bacterial surface protein having a molecular weight of about 190 kDa, and that carry a collagen binding protein CBP (Collagen Binding Protein, also known as Cnm) having a molecular weight of about 120 kDa, and also discovered that all these virulent bacterial strains have low cell surface charge. The influences of S. mutans on hemorrhage has never been reported so far, and the findings that particular strains of S. mutans exacerbate the prognosis of hemorrhage and that PA and CBP as well as cell surface charge are involved in such virulence were therefore surprising results. Based on these findings, the inventors further proceeded with the study, and found that CBP-positive bacterium has an ability to inhibit platelet aggregation, thereby completed the invention.

Accordingly, the present invention relates to a method of detecting a hemorrhage aggravating oral bacterium, the method comprising detecting PA and/or CBP and/or cell surface charge of oral bacteria in a sample, wherein the presence of the hemorrhage aggravating oral bacterium is determined if PA is not detected and/or CBP is detected and/or the cell surface charge is negative.

Moreover, the present invention relates to a method of screening a subject at a high risk of hemorrhage aggravation, the method comprising detecting PA and/or CBP and/or cell surface charge of oral bacteria in a biological sample obtained from the subject, wherein a high risk of hemorrhage aggravation is determined if PA is not detected and/or CBP is detected and/or the cell surface charge is negative

Alternatively, the present invention relates to a method of judging the risk of hemorrhage aggravation in a subject, the method comprising detecting PA and/or CBP and/or cell surface charge of oral bacteria in a biological sample obtained from the subject, wherein a high risk of hemorrhage aggravation in the subject is determined if PA is not detected and/or CBP is detected and/or the cell surface charge is negative.

Furthermore, the present invention relates to any one of said methods wherein the hemorrhage is hemorrhage by diabrosis.

The present invention also relates to any one of said methods wherein the oral bacterium is Streptococcus mutans.

The present invention further relates to any one of said methods wherein PA is selected from the group consisting of:
(1) a polypeptide comprising an amino acid sequence according to SEQ ID NO. 1, 17, 19, 21 or 23;
(2) a polypeptide comprising one or more mutations in the polypeptide of (1) but having an equal function to the polypeptide of (1);
(3) a polypeptide comprising an amino acid sequence encoded by a nucleic acid sequence that hybridizes with a nucleic acid sequence according to SEQ ID NO. 2, 18, 20, 22 or 24 or its complementary sequence or its fragment under stringent condition, and having an equal function to the polypeptide of (1); and
(4) a polypeptide comprising an amino acid sequence having 70% or more homology with an amino acid sequence according to SEQ ID NO. 1, 17, 19, 21 or 23.

The present invention further relates to any one of said methods wherein PA comprises a polypeptide consisting of an amino acid sequence according to SEQ ID NO. 1, 17, 19, 21 or 23.

The present invention also relates to any one of said methods wherein CBP is selected from the group consisting of:
(1) a polypeptide comprising an amino acid sequence according to SEQ ID NO. 5, 9, 27 or 31;
(2) a polypeptide comprising one or more mutations in the polypeptide of (1) but having an equal function to the polypeptide of (1);
(3) a polypeptide comprising an amino acid sequence encoded by a nucleic acid sequence that hybridizes with a nucleic acid sequence according to SEQ ID NO. 6, 10, 28 or 32 or its complementary sequence or its fragment under stringent condition, and having an equal function to the polypeptide of (1);
(4) a polypeptide comprising an amino acid sequence having 70% or more homology with an amino acid sequence according to SEQ ID NO. 5, 9, 27 or 31.

The present invention also relates to any one of said methods wherein CBP comprises a polypeptide consisting of an amino acid sequence according to SEQ ID NO. 5, 9, 27 or 31.

Also, the present invention relates to a reagent for detection of a hemorrhage-aggravating oral bacterium, the reagent comprising an oral bacterial PA-detecting agent and/or CBP-detecting agent.

Furthermore, the present invention relates to an oral bacterial PA-specific antibody for detection of a hemorrhage-aggravating oral bacterium.

Moreover, the present invention relates to a kit for detection of a hemorrhage-aggravating oral bacterium and/or for screening of a subject at a high risk of hemorrhage aggravation and/or for determination of the risk of hemorrhage aggravation in the subject, the kit comprising at least:

a PA-detecting reagent, and

a CBP-detecting reagent.

Also, the present invention relates to a hemostatic agent comprising PA protein of an oral bacterium or nucleic acid encoding the PA protein.

Also, the present invention relates to an inhibitor of platelet aggregation comprising a substance that binds to an oral bacterium PA protein or to a nucleic acid encoding the PA protein.

Also, the present invention relates to a hemorrhage aggravation inhibitor comprising a substance that binds to an oral bacterium CBP or to a nucleic acid encoding the CBP protein.

Alternatively, the present invention relates to an agent for detecting collagen-denuded site in tissue comprising CBP of an oral bacterium.

Also, the present invention relates to a carrier for delivering a substance to the collagen-denuded site comprising CBP of an oral bacterium.

The present invention also relates to a therapeutic agent for hemorrhage comprising CBP of an oral bacterium and a hemostatic agent.

Moreover, the present invention relates to said therapeutic agent for hemorrhage for a subject having low sensitivity of platelet to collagen.

Also, the present invention relates to a prophylactic agent for hemorrhage aggravation comprising an oral bacterium-removing agent.

### THE EFFECTS OF THE INVENTION

The present invention allows rapidly and easily diagnosing the risk of causing hemorrhage aggravation in a subject. Also, the method of the present invention enables detecting responsible factors of hemorrhage aggravation using readily-available biological samples such as saliva and plaque without employing any special analyzers. As such, the present invention allows to specify a high-risk population of hemorrhage aggravation, to treat the individuals belonging to this population with a regimen such as removing virulent bacteria and advising dental hygiene, and thereby to effectively prevent a hemorrhage aggravation.

### BRIEF DESCRIPTION OF THE DRAWINGS

FIG. 1 is a flow-chart of a system to detect a S. mutans strain which may cause hemorrhage aggravation.

FIG. 2 is a scheme of methods of culturing and detecting S. mutans.

FIG. 3 is a scheme of methods of detecting PA-deleted S. mutans and CBP-carrying S. mutans.

FIG. 4 is a diagram showing the results of infecting mice with S. mutans strains and inducing an aggravation of cerebral hemorrhage. (a-b) Representative macro images of (a) mouse whole blains and (b) coronal slices 24 hours after inducing cerebral hemorrhage and administering either PBS (control) or S. mutans TW295 strain. Arrowheads indicate the breeding sites. (c) A graph showing the differences in breeding areas between various S. mutans strains used for infection. Each column represents the mean±SEM from 10 to 21 independent experiments (*P<0.05, **P<0.01). (d) Typical microscopic photographs of the vessel-damaged hemisphere from the control or TW295-administered mouse isolated 3 hours after the induction of hemorrhage. The enlarged view indicates microvascular hemorrhages. (e) Samples were collected 24 hours after administration of the bacteria, and the MMP-9 activity was detected by gelatin gel zymography. The MMP-9 activity was consistently high in the vessel-damaged hemisphere in TW295-administered group compared with those in the control group (Ips: vessel-damaged hemisphere of the brain, Ctr: contralateral hemisphere).

FIG. 5 is a diagram showing the accumulation of administered bacteria to the damaged vessel and their interaction with collagen surrounding the vessel. (a) 24 hours after infecting mice with TW295 strain, cellular DNA of the infected bacteria was detected in each organ by PCR using specific primers. Labels on each lane is: M: molecular marker (100 by ladder), Std: positive control (genomic DNA extracted from TW295 strain), Ips: vessel-damaged hemisphere of the brain, Ctr: contralateral hemisphere of the brain, respectively. (b) Tissues isolated from damaged and undamaged area are plated onto culture dishes to harvest S. mutans. Germ numbers of isolated bacteria are expressed in CFU/mg protein. (c) Representative in vivo scanning electron microscopic images showing interaction between an injured blood vessel and infecting bacterial cells observed in a sample prepared from cerebral tissue resected 3 hours after the administration of TW295 strain. The right panel is enlarged image of the boxed part. White arrowheads indicate leaking of accumulated bacteria at the damaged site. (d) An in vivo SEM image showing the interaction between the bacteria and collagen fibers surrounding the vessel in damaged hemisphere. White arrowheads indicate the bacteria interacting with collagen fibers.

FIG. 6 is a diagram showing the detection of Gram staining of bacteria accumulating in the damaged brain area. White arrowheads indicate accumulated bacteria. Scale bar: 25 μm.

FIG. 7 is a diagram summarizing the effects of the presence of collagen binding protein (CBP) and the expression of protein antigen (PA) on the virulence.

FIG. 8 is a diagram showing the impacts of CBP-expressing S. mutans on collagen binding activity and cerebral hemorrhage. (a) Collagen binding activities of various S. mutans strains were assessed under certain condition using 2 mg Type I collagen and 1×10¹⁰ bacterial cells. The result from each strain was expressed as a percentage to that from TW871 strain. (b) The areas of hemorrhage in mice infected either with TW295, a TW295CND strain generated from TW295, or a MT8141PD strain generated from MT8141 or MT8141. Each column represents the mean ±SEM from 11 to 16 independent experiments. (c) Correlation between collagen binding activity and total hemorrhage area.

FIG. 9 is a diagram showing the relationship between collagen binding activity and platelet aggregation in various S. mutans strains. (a) Platelet aggregation activity of various S. mutans strains. The assay was performed using mouse whole blood and an aggregometer under certain conditions using 4 μg collagen and 1×10⁷ bacterial cells. The result from each strain was expressed as a percentage to that of the case wherein collagen was included but no bacterial cells were included. (b) Correlation between collagen binding activity and platelet aggregation.

FIG. 10 is a diagram showing the effects of various S. mutans strain on platelet aggregation. (a) Platelet aggregation rates after the addition of either standard strain MT8148 or virulent strain TW871 in various cell numbers. Aggregation in mouse whole blood was measured after the addition of S. mutans and expressed as a percentage. (b-c) Platelet aggregation rates for 58 clinical strains were assessed by the same method as (a). Results are expressed (b) by their serotypes (c; n=20, e; n=15, f; n=10, and k; n=13), or (c) by the sources of the isolated strains (oral cavity; n=45, blood; n=13). White and black circles indicate the strain isolated from oral cavity and blood, respectively. Bold horizontal bars indicate the mean value of each group (*p<0.05, **p<0.01, ***p<0.001). (d) A typical chart of the platelet aggregation assay using human platelet-rich plasma. MT8141 or TW871 bacterial cells (10⁷ CFU) and human platelet-rich plasma were incubated, then collagen (4 μg) was added after 5 minutes. (e) The effect of bacteria on arachidonic acid-induced platelet aggregation. In whole blood aggregation, collagen was substituted by arachidonic acid as aggregating reagent. Each column represents the mean±SEM of 8 to 14 independent experiments.

FIG. 11 is a diagram showing the relationship between the difference in bacterial cell surface conditions and collagen-induced platelet aggregation. (a) Representative observation of scanning electron microscopy of the reaction of S. mutans strain and platelets to collagen. Platelet fraction was collected after reacting with collagen, and observed with scanning electron microscope. (b) Zeta potential values of S. mutans strain. Zeta potential values of the standard strain MT8148 and its isogenic mutant strain MT8148PD were measured and expressed in mV. Moreover, 7 oral cavity-isolated strains and 7 blood isolated strains were subjected to the measurement. Each column represents the data from 3 to 5 independent experiments. (c) Transmission electron microscopy observation of bacterial surface. (d) Scanning electron microscopy observation of bacterial surface.

FIG. 12 is a diagram showing correlation of platelet aggregation and zeta potential value. Each point represents one bacterial strain.

FIG. 13 is a diagram showing three-dimensional reconstructed images of the bacterial surface. Bacterial membranes were compared using three-dimensional reconstructed images generated by TEM computerized tomography. (a) MT8148 peptidoglycan layer is observed as a transparent and very smooth layer in the three-dimensional image obtained by TEM. (b) The bacterial surface peptidoglycan of the virulent strain TW295 is opaque and its outer shape is obscure (arrowheads).

FIG. 14 is a diagram showing the effects of the bacteria isolated from human stroke patients on collagen binding activity and platelet aggregation. (a) Collagen binding activity of S. mutans strains isolated from stroke patients (SMH2 and SMH4). The activity was assessed under certain conditions using 2 mg Type I collagen and 1×10¹⁰ bacterial cells. The result for each strain is expressed as a percentage to that for TW871. (b) Platelet aggregation activity of S. mutans strains isolated from stroke patients. Assay was performed by impedance method on aggregometer using mouse whole blood under certain condition using 4 pg Type I collagen and 1×10⁷ bacterial cells. The result for each strain is expressed as a percentage to that for the case where collagen was added but no bacterial cells are added.

FIG. 15 is a diagram showing the effects of CBP-expressing S. mutans isolated from stroke patients on cerebral hemorrhage. (a) A representative macro image of whole brain of a mouse to which SMH4 isolated from a stroke patient was administered, 24 hours after the onset of cerebral hemorrhage. (b) Representative macro images of brain sections of a mouse to which SMH4 was administered. (c) Sizes of cerebral hemorrhage regions in groups of mice infected with CBP-expressing S. mutans isolated from stroke patients (SMH2 and SMH4). Each column represents the mean±SEM of 11 to 16 independent experiments (*p<0.05).

FIG. 16 is a schematic diagram showing a putative mechanism of aggravation of cerebral hemorrhage by S. mutans cells. (a) Normal hemostasis induced by platelet aggregation at damaged site of an endothelial cell. (b) S. mutans cells with a high negative charge accumulate onto denuded collagen with a positive charge. Moreover, S. mutans cells carrying collagen binding protein have a high affinity to denuded collagen. Both these factors result in an activation of MMP-9 and inhibition of platelet aggregation at the damaged site of the endothelial cell, thereby causing a sustained bleeding.

FIG. 17 is a schematic diagram of the experimental protocols to photochemically induce damage onto mouse mesencephalic artery endothelial cells. (a) A schematic diagram of the hypothesis and the experimental protocols. (b) Time schedule of the experimental procedures.

FIG. 18 is a diagram showing examples of the results of the analysis to determine the presences of PA-deleted S. mutans and CBP-carrying S. mutans.

FIG. 19 is a graph showing the results of the investigation on optimum conditions for culturing S. mutans (culture in an aerobic/anaerobic condition, concentration of antibiotics, nutrient concentration).

FIG. 20 is a graph showing the results of the investigation on the possible stock period for saliva to be used for the detection of virulent S. mutans.

### DESCRIPTION OF EMBODIMENTS

The present invention provides a method of detecting a hemorrhage-aggravating oral bacterium, the method comprising detecting PA and/or CBP and/or cell surface charge of oral bacteria in a sample, wherein the presence of the hemorrhage-aggravating oral bacterium is determined by that PA is not detected and/or that CBP is detected and/or that the cell surface charge is negative.

The present invention provides, in another embodiment, a method of screening a subject at a high risk of hemorrhage aggravation, the method comprising detecting PA and/or CBP and/or cell surface charge of oral bacteria in a biological sample obtained from a subject, wherein a high risk of hemorrhage aggravation is determined by that PA is not detected and/or that CBP is detected and/or that the cell surface charge is negative.

The present invention further provide, in another embodiment, a method of determining the risk of hemorrhage aggravation in a subject, the method comprising detecting PA and/or CBP and/or cell surface charge of oral bacteria in a biological sample obtained from a subject, wherein a high risk of hemorrhage aggravation is determined in the subject by that PA is not detected and/or that CBP is detected and/or that the cell surface charge is negative.

A mutans streptococci Streptococcus mutans, an oral bacterium that is a major pathogenic bacteria of dental caries, are known to have four serotypes (c, e, f and k). S. mutans is also known to be a pathogenic bacterium of bacteremia and infective endocarditis, and reported to be relevant to cardiovascular diseases since bacterial DNA of S. mutans was detected from the specimens of cardiac valve and aortic aneurysm (Nakano et al., 2008, Japanese Dental Science Review, 44: 29-37). However, association of S. mutans to other diseases, for example its impact on cerebrovascular diseases, have never been investigated so far.

Studies by the inventors disclosed herein revealed that the intravenous administration of some of different S. mutans strains inhibits spontaneous hemostatic action and induces aggravation of hemorrhage, when mild cerebral hemorrhage has been induced by damaging the middle cerebral artery. A MT8148 strain generally isolated from the oral cavity (serotype (Minami et al., 1990, Oral Microbiol. Immunol., 5: 189-194) does not cause such effects, thought there are strains among serotype k that evokes hemorrhage aggravation. In particular, TW295 strain and TW871 strain (Nakano et al., 2004, Journal of Clinical Microbiology, 42(1); 198-202), SA53 strain (Nakano et al., 2007, J. Clin. Microbiol., 45: 2614-2625), and LJ32 strain (Nakano, K. et al., 2008, J. Dent. Res. 87: 964-968) cause a significant hemorrhage aggravation.

The inventors found that those highly virulent S. mutans strains lack PA, a major bacterial surface protein. The inventors also found that among the PA-deficient strains, the virulence of the strains carrying CBP, another bacterial surface protein, was particularly high. The inventors further confirmed that TW295 strain-like hemorrhage aggravation is not exhibited when CBP-encoding gene of TW295 strain has been deleted by genetic engineering; and that a strain in which PA-encoding gene has been deleted from MT8148 strain exhibits hemorrhage aggravation, confirming that CBP and PA are involved in hemorrhage aggravating activity of S. mutans. The inventors further found that CBP-carrying S. mutans strains are detected in the oral cavity of human patients with hemorrhagic stroke, and further confirmed that CBP-carrying S. mutans strains isolated from such patients cause aggravation of cerebral hemorrhage in vivo. The inventors further found that the cell surface charge of a highly virulent S. mutans strain is negative. Based on these findings, the inventors demonstrated that these bacterial surface protein and cell surface charge can be utilized as useful markers for detection of a S. mutans strain that exacerbates hemorrhage, for screening of a subject at a high risk of hemorrhage aggravation, and for determination of the risk of hemorrhage aggravation of a subject.

The oral bacterium detected according to the method of the present invention may exacerbate any bleeding, though, in particular, would exacerbate a hemorrhage by diabrosis caused by the occurrence of damage on the vascular wall due to a traumatic injury, an ulcer or a ruptured aneurysm. Representative examples of hemorrhage by diabrosis include such as cerebral hemorrhage (intracerebral hemorrhage, subarachnoid hemorrhage, chronic subdural hematoma), bleeding due to traumatic injury or compression, hemorrhage after delivery, subcutaneous hemorrhage associated with diseases. Also, diseases which cause bleeding tendency include connective tissue disorders (such as allergic purpura), thrombocytopenia (such as disseminated intravascular coagulation and aplastic anemia) or platelet disorders (such as thrombasthenia), or disorders in coagulation system (such as coagulation disorders associated with liver diseases and vitamin K deficiency). Endogenous or exogenous circulating anti-coagulation substances (such as lupus anticoagulant and VIII factor anti-coagulation substance) may also cause bleeding tendency.

Hemorrhage aggravation herein means that the spontaneous hemostatic action against bleeding caused by such endogenous or exogenous factor is delayed, decreased or lost as compared to a normal subject. Also, a subject at a high risk of hemorrhage aggravation means that, in said subject, the spontaneous hemostatic action by platelets is highly likely to be delayed, decreased or lost as compared to a normal subject upon the bleeding due to an endogenous or exogenous factor.

PA (Protein Antigen) is a surface protein of approximately 190 kDa found in MT8148 strain, a S. mutans wild-type strain, and also known in various other names such as PAc (Protein Antigen c), SpaP, Antigen I/II and Antigen B, P1 and MSL-1. PA polypeptide comprises 3 alanine-rich repeat domains (A-region) at N-terminal side and 3 proline-rich repeat domains (P-region) at central part, and has cell wall/membrane-spanning domain at C-terminal. It has been reported that the A-regions are involved in the attachment of bacterial cells to teeth (Matsumoto-Nakano et al., 2008, Oral Microbiology and Immunology, 23:265-270). Also, there have been reports that PA is involved in infective endocarditis by S. mutans (Nakano et al., 2008, Japanese Dental Science Review, 44: 29-37); that an antibody against PA inhibits the attachment of bacterial cells to a hydroxyapatite substrate (Kawato et al., 2008, Oral Microbiology and Immunology, 23:14-20); and that an antiserum against PA is useful as a vaccine for dental caries (Okahashi et al., 1989, Molecular Microbiology, 3(2): 221-228). Although there is a region between A-region and P-region of PA, in which amino acid sequences are highly variable between strains (for example, in MT8148 strain, residues from 679 to 827), the repeat domain and transmembrane domain are highly conserved among strains.

Also, it is reported that strains of serotype k, which are often detected in patients with infective endocarditis, lacks PA in a high percentage, and that both the hydrophobicity of the bacterial body sensitivity to phagocytosis are low in this serotype (Nakano et al., 2008, Journal of Dental Research, 87(10): 964-968).

Known PA includes, for example, PA of serotype c MT8148 (DDBJ Accession No. X14490, amino acids: SEQ ID NO. 1, nucleic acids: SEQ ID NO. 2), PA of LJ23 strain (DDBJ Accession No. AB364261, amino acids: SEQ ID NO. 17, nucleic acids: SEQ ID NO. 18), PA of SA98 strain (DDBJ Accession No. AB364285, amino acids: SEQ ID NO. 19, nucleic acids: SEQ ID NO. 20), as well as spaP gene of antigen I/II (DDBJ Accession No. X17390, Kelly et al., 1989, FEBS Lett. 258(1), 127-132, amino acids: SEQ ID NO. 21, nucleic acids: SEQ ID NO. 22) and a meningococcus Neisseria meningitidis iron binding protein fbp gene (X53469, Berish et al., 1990, Nucleic Acid Research, 18(15): 4596-4596, amino acids: SEQ ID NO. 23, nucleic acids: SEQ ID NO. 24).

CBP, i.e., another anchor protein of S. mutans (also denoted as Cnm), is a Type I collagen binding protein of approximately 120 kDa molecular weight, and has a collagen binding domain (CBD, residues from 152 to 316), B repeat domain (residues from 328 to 455) and LPXTG motif (residues from 507 to 511) (Sato et al., 2004, Journal of Dental Research, 83(7): 534-539). CBP gene-carrying frequency of S. mutans is about 10 to 20%, and CBP-positive strain is predominantly expressed in serotype f and k (Nakano et al., 2007, J. Clin. Microbiol., 45: 2616-2625).

The studies by the inventors revealed that, for CBP of serotype k TW295 strain (DDBJ Accession No. AB102689, amino acids: SEQ ID NO. 3, nucleic acids: SEQ ID NO. 4), CBD (amino acids: SEQ ID NO. 5, nucleic acids: SEQ ID NO. 6) and LPXTG motif are highly conserved between strains, whereas the number of repeats in the B repeat domain varies between strains (Nomura et. al., 2009, J. Med. Microbiol., 58:469-75).

In one embodiment of the present invention, PA is defined as:
(1) a polypeptide comprising an amino acid sequence expressed by SEQ ID NOs. 1, 17, 19, 21 or 23;
(2) a polypeptide comprising one or more, preferably 1 to 20, 1 to 15, 1 to 10, or one or several mutations in polypeptide of (1), but having an equal function as the polypeptide of (1);
(3) a polypeptide comprising an amino acid sequence encoded by a nucleic acid sequence that hybridizes under stringent condition with a nucleic acid sequence expressed by SEQ ID NOs. 2, 18, 20, 22 or 24 or its complementary sequence or its fragment, and having an equal function as the polypeptide of (1); or
(4) a polypeptide comprising an amino acid sequence having 70% or more, preferably 80% or more, more preferably 90% or more, still more preferably 95% or more homology to an amino acid sequence expressed by SEQ ID NOs. 1, 17, 19, 21 or 23, and having an equal function as the polypeptide of (1).

Preferably, PA comprises a polypeptide consisting of an amino acid sequence expressed by SEQ ID NOs. 1, 17, 19, 21 or 23. More preferably, PA comprises a polypeptide consisting of an amino acid sequence expressed by SEQ ID NO. 1.

PA that can be used in the method of the present invention may be a polypeptide comprising one or more amino acid mutations (deletions, substitutions and/or additions), as long as it comprises an amino acid sequence encoded by a nucleic acid sequence that hybridizes under stringent condition with a nucleic acid sequence expressed by SEQ ID NOs. 2, 18, 20, 22 or 24 (nucleic acid encoding the PA protein sequence) or its complementary sequence or its fragment, and has a equal function as a polypeptide comprising an amino acid sequence expressed by SEQ ID NOs. 1, 17, 19, 21 or 23 (amino acid sequence of PA protein). Mutations may be naturally occurring mutations or mutations generated by any known procedures, e.g., cleavage or insertion of a nucleic acid by restriction enzyme, site-specific mutagenesis, or radiation or ultraviolet irradiation. Moreover, the number of mutated amino acids may be 1 to 20, 1 to 15, 1 to 10, or 1 to several, for example.

Furthermore, in one embodiment of the present invention, CBP is defined as:
(1) a polypeptide comprising an amino acid sequence expressed by SEQ ID NOs. 5, 9, 27 or 31;
(2) a polypeptide comprising one or more, preferably 1 to 20, 1 to 15, 1 to 10, or one or several mutations in the polypeptide of (1), but having an equal function as the polypeptide of (1);
(3) a polypeptide comprising an amino acid sequence encoded by a nucleic acid sequence that hybridizes under stringent condition with a nucleic acid sequence expressed by SEQ ID NOs. 6, 10, 28 or 32 or its complementary sequence or its fragment, and an equal function as the polypeptide of (1); or
(4) a polypeptide comprising an amino acid sequence having 70% or more, preferably 80% or more, more preferably 90% or more, still more preferably 95% or more homology with an amino acid sequence expressed by SEQ ID NOs. 5, 9, 27 or 31, and having an equal function as the polypeptide of (1).

CBP polypeptide that can be used in the method of the present invention may be a polypeptide comprising one or more, e.g., 1 to 20, 1 to 15, 1 to 10, or one or several amino acid mutations (deletions, substitutions and/or additions), as long as it comprises an amino acid sequence encoded by a nucleic acid sequence that hybridizes under stringent condition with a nucleic acid sequence expressed by SEQ ID NOs. 6, 10, 28 or 32 (nucleic acid sequence encoding CBD of S. mutans TW295, TW871, SA53 or LJ32 strains) or its complementary sequence or its fragment, and has an equal function as a polypeptide comprising an amino acid sequence expressed by SEQ ID NOs. 5, 9, 27 or 31 (CBD amino acid sequence of S. mutans TW295, TW871, SA53 or LJ32 strain).

For instance, CBP polypeptide may be a polypeptide comprising an amino acid sequence encoded by a nucleic acid sequence that hybridizes under stringent condition with a nucleic acid sequence expressed by SEQ ID NOs. 4, 8, 26 or 30 (a nucleic acid sequence encoding CBP of S. mutans TW295 strain, TW871 strain (DDBJ Accession No. AB469914), SA53 strain (AB465299) or LJ32 strain (AB465263)) or its complementary sequence or its fragment, and has an equal function as a polypeptide comprising an amino acid sequence expressed by SEQ ID NOs. 3, 7, 25 or 29 (an amino acid sequence of CBP protein of S. mutans TW295, TW871, SA53 or LJ32 strain).

Preferably, CBP comprises a polypeptide consisting of an amino acid sequence expressed by SEQ ID NOs. 5, 9, 27 or 31.

Whether a PA or CBP mutant has an equal function as PA or CBP or not may be confirmed using any known means. For instance, the ability of PA mutant making the bacterial cell adhere to a hydroxyapatite substrate may be determined by raising a specific antibody against the mutant peptide by a known method, and assaying the inhibition of adhesion of bacteria to the hydroxyapatite by said antibody according to a method described in Kawato et al., 2008, Oral Microbiology and Immunology, 23:14-20. Alternatively, the biding ability of a CBP mutant to Type I collagen may be determined by collagen binding assay described in Nomura et al., 2009, J. Med. Microbiol., 58(4): 469-475. By such means, the ability of a mutant can be assessed in comparison with an appropriate negative control, or with PA or CBP as a positive control. For instance, certain mutant is considered as a functional mutant when at least one function described above is better, e.g., 10% or better, 25% or better, 50% or better, 75% or better, or even 100% or better, than the negative control, and/or when said function is 1/100 or less, 1/50 or less, 1/25 or less, 1/10 or less, ⅕ or less, or even ½ or less, than the positive control.

In the method of the present invention, the surface charge of a bacterial cell can be measured by any known method, e.g., zeta potential measuring method. Zeta potential, also called as electrokinetic potential, is a potential difference that arises on the interface between a solid and a liquid contacting to each other in a relative motion, which may be used as an index for the surface charge of a bacterial cell. Zeta potential can be calculated from electrophoretic mobility of bacterial cells using an equation of Smoluchowski:

ζ=ηu/ε0εr

wherein, ζ indicates the zeta potential, η indicates the viscosity of the solvent, u indicates the electrophoretic mobility, ε0 indicates the dielectric constant of a vacuum, εr indicates the dielectric constant of the solvent.

Methods of electrophoresis suitable for measuring zeta potential are not particularly limited as long as it can measure the migrating speed of bacterial cells, and include, for example, capillary electrophoresis, microscopic electrophoresis, rotating diffraction gating method and laser Doppler electrophoresis.

In the method of the present invention, a negative surface charge of the bacterial cell is an index for a highly virulent oral bacterium, and is a criterion for the presence of a hemorrhage-aggravating oral bacterium and a risk of hemorrhage aggravation. Namely, collagen fibers denuded within a damaged vessel are positively charged, and if bacterial cell surface is negatively charged, the bacterial cell may easily interact with denuded collagen fibers, thereby resulting in hemorrhage aggravation due to the inhibition of platelet aggregation. Typically, an oral bacterium is determined to be highly virulent when the surface charge measured as zeta potential is −1.0 mV or below, more preferably −3.0 mV or below, still more preferably −4.0 mV or below, even more preferably −5.0 mV or below, particularly preferably −8.0 mV or below.

In aforementioned methods of the present invention, oral bacterial PA, CBP and cell surface charge may be used either alone or in combination. Accordingly, either PA alone, CBP alone, or cell surface charge alone may be detected, or any combination of PA, CBP and cell surface charge, namely, both PA and CBP, both PA and cell surface charge, both CBP and cell surface charge, or, all of PA, CBP and cell surface charge may be detected. Furthermore, each of the criteria, i.e., that PA is not detected, that CBP is detected and that the cell surface charge is negative, may be used alone or in combination, according to the items to be detected.

Major bacteria species that are identified as hemorrhage-aggravating oral bacteria include mutans streptococci such as Streptococcus mutans, Streptococcus sobrinus, Streptococcus cricetus, Streptococcus rattus, Streptococcus downei, Streptococcus sanguinis, Streptococcus oralis, Streptococcus gordonii, and Streptococcus salivarius. Particularly, S. mutans TW295 strain, TW871 strain, SA53 strain, and LJ32 strain would cause severe hemorrhage aggravation.

Screening of other bacteria that could induce hemorrhage aggravation can be carried out utilizing databases such as NCBI GenBank®, DDBJ (DNA Data Bank of Japan, http://www.ddbj.nig.ac.jp/) and EMBL, and publicly available search tools such as BLAST.

The present invention provides, in one embodiment, a reagent for the detection of a hemorrhage-aggravating oral bacterium comprising an oral bacterial PA detecting agent and/or an oral bacterial CBP detecting agent.

In one embodiment, the PA detecting agent comprises an oral bacterial PA-specific antibody. Using the PA-specific antibody developed by the inventors, the presence or absence of a highly virulent S. mutans can rapidly and easily detected. The PA-specific antibody is preferably an antibody or its fragment induced from polypeptide comprising an amino acid sequence of SEQ ID NO. 1 or its immunogenic fragment. Alternatively, the PA-specific antibody may be an antibody or its fragment induced from a polypeptide having 70% or more, preferably 80% or more, more preferably 90% or more, still more preferably 95% or more homology with an amino acid sequence of SEQ ID NOs. 1, 17, 19, 21 or 23, and having an immunogenicity to induce an antibody production against a polypeptide comprising an amino acid sequence of SEQ ID NOs. 1, 17, 19, 21 or 23. For example, a recombinant PA comprising the polypeptide (see, e.g., Nakano et al., 2006, Microbes and Infection, 8:114-121) may be used as an antigen to produce a monoclonal or polyclonal antibody.

In one embodiment, CBP detecting agent comprises a substrate (such as a microplate, test tube or slide glass) coated with Type I collagen. The binding affinity of CBP to Type I collagen (Nomura et al., 2009, J. Med. Microbiol., 58(4): 469-475) can be utilized to allow CBP-expressing bacterial cell to attach a substrate coated with Type I collagen, which can easily be detected.

In another embodiment, the CBP detecting agent comprises a specific antibody against an oral bacterial CBP. The CBP-specific antibody may be a specific antibody against the collagen binding domain of CBP, preferably, an antibody or its fragment induced from a polypeptide comprising an amino acid sequence of SEQ ID NOs. 5, 9, 27 or 31 or its immunogenic fragment. Alternatively, the CBP-specific antibody may be an antibody or its fragment induced from a polypeptide having 70% or more, preferably 80% or more, more preferably 90% or more, still more preferably 95% or more homology with an amino acid sequence of SEQ ID NOs. 5, 9, 27 or 31, and having an immunogenicity to induce an antibody production against a polypeptide comprising an amino acid sequence of SEQ ID NOs. 5, 9, 27 or 31.

In the present invention, the antibody fragment comprises, for example, without limitation, various functional fragments such as Fab, Fab′, F(ab′)2, scFv, dsFv (disulfide-stabilized V region fragment), and CDR-containing fragment.

The present invention provides, in one embodiment, a kit for the detection of a hemorrhage-aggravating oral bacterium, and/or for the screening of a subject at a high risk of hemorrhage aggravation, and/or for the determination of the risk of hemorrhage aggravation in a subject. The kit comprises at least a PA-detecting reagent and a CBP-detecting reagent.

In one embodiment, the kit comprises as a PA-detecting reagent an oral bacterial PA-specific antibody.

In one embodiment, the kit comprises as a CBP-detecting reagent a substrate coated with Type I collagen (such as a microplate, test tube or slide glass).

In another embodiment, the kit comprises as a CBP-detecting reagent a CBP-specific antibody.

The kit of the present invention may further comprise one or more of the followings for culturing S. mutans:
An instrument for collecting saliva such as a spitz for collecting saliva (the material and shape is not particularly limited as long as it is sterilized and suitable for collecting and seeding).
A collecting instrument such as a dropper capable of collecting saliva of approximately 10 μl.
S. mutans selection medium (Special Medium A). For example, sterile substrate coated with MSB agar medium (Mitis-salivariusagar medium (e.g., Difco Laboratories) supplemented with an antibiotic (e.g., bacitracin SIGMA-ALDRICH)) and sucrose (e.g., Wako Pure Chemical Industries, Ltd.)). The substrate is not particularly limited as long as it is such as a dish or well plate, though typically a plate of about 24-well (e.g., 24 well with Lid MICROPLATE (IWAKI)) is used. Bacitracin is preferably used at about 100 unit/ml. Sucrose is preferably used at about 15%.
A sealing and/or deoxygenating instrument for culturing under an anaerobic condition such as Anaero Pack® or a CO2 chamber.
A sterile stick for picking up bacterial colonies (such as a toothpick or tip).
A liquid medium for culturing the picked-up colonies (Special Medium B). For example, sterilized Brain Heart Infusion (BHI) liquid medium (Difco Laboratories) contained in a disposable test tube.

The kit of the present invention may further contain one or more of the followings for detecting S. mutans:
A collecting instrument suitable for collecting bacterial solution of approximately 10 μl such as a dropper.
A special medium for detecting S. mutans (Special Medium C). For example, sucrose (Wako Pure Chemical Industries, Ltd.)-containing BHI solution 100 μl added to a substrate. The substrate is not particularly limited as long as it is such as a well plate or test tube, though typically a 96-well plate (e.g., MULTI WELL PLATE for ELISA (SUMIRON)) is used. Sucrose is used at about 1%.
A wash buffer (Wash Buffer A: PBS solution or sterile water may be used, though preferably PBS solution is used.)
A Gram-positive bacteria detecting reagent (Buffer 1: for example, a solution in which to sterile distilled water about 0.5% crystal violet (e.g., Wako Pure Chemical Industries, Ltd.) is added as the Gram-positive bacteria detecting reagent.)
A mordanting reagent (Buffer 2: a suitable mordanting reagent may be selected depending on the bacteria detecting reagent. For example, 7% acetate (e.g., Wako Pure Chemical Industries, Ltd.) solution or sterile water may be used for crystal violet, though preferably acetate solution is used.)

The kit of the present invention may further comprise one or more of the followings for detecting PA-deleted S. mutans:
A plate for detecting PA-deleted S. mutans. It is not particularly limited as long as sterile it is a well plate, though typically a 96-well plate (e.g., MICROTEST U-Bottom (BECTON DICKINSON)) is used.
A wash buffer (Wash Buffer B: a solution in which to PBS solution or sterile water about 0.05% of a surfactant such as Triton X-100 (e.g., Wako Pure Chemical Industries, Ltd.) are added. Preferably PBS solution is used.)
A buffer (Buffer 3: a mixture of Tris buffered saline (pH6.8), 100 mM dithiothreitol (e.g., Wako Pure Chemical Industries, Ltd.) and 20% glycerin (e.g., Wako Pure Chemical Industries, Ltd.).)
A blocking solution (Buffer 4: a PBST solution containing approximately 5% of skimmed milk (e.g., BECTON DICKINSON).)
A primary antibody (Buffer 5: a PBST solution containing approximately 0.1% of anti-PA antiserum.)
A secondary antibody (Buffer 6: a PBST solution containing approximately 0.1% of a primary antibody against the immunoglobulin (e.g., Dakopatts).)
A color-developing reagent (Buffer 7: AP (100 mM 2-amino-2-hydroxymethyl-1,3-propanediol, 5 mM magnesium chloride, 100 mM sodium chloride) buffer supplemented with NBT solution (Wako Pure Chemical Industries, Ltd.) at final concentration of 0.6% and BCIP solution (Wako Pure Chemical Industries, Ltd.) at final concentration of 0.33%.)

The kit of the present invention may further comprise one or more of the followings for detecting CBP-carrying S. mutans:
A special medium for detecting CBP-carrying S. mutans (Special Medium D: a mixed solution of 0.6% acetate-containing sterile distilled water and Type I collagen (Sigma) in 9:1 ratio contained in the Special Plate used in Analysis 3.)
A wash buffer (Wash Buffer A: PBS solution or sterile water may be used, though preferably PBS solution is used.)
A buffer (Buffer 8: Wash Buffer A containing approximately 5% bovine albumin (Sigma).)
A wash buffer (Wash Buffer C: PBS solution or sterile water containing a surfactant such as approximately 0.01% Tween 20 (Wako Pure Chemical Industries, Ltd.). Preferably, PBS solution is used.)
A fixative solution (Buffer 9: for example, sterile distilled water containing approximately 25% formaldehyde (Wako Pure Chemical Industries, Ltd.).)
A Gram-positive bacteria detecting reagent (e.g., above Buffer 1: a solution in which to sterile distilled water approximately 0.5% crystal violet (Wako Pure Chemical Industries, Ltd.) is added as a Gram-positive bacteria detecting reagent.)
A mordanting reagent (e.g., above Buffer 2: 7% acetate (e.g., Wako Pure Chemical Industries, Ltd.) solution or sterile water may be used, though preferably acetate solution is used.)

A skilled person in the art may appropriately adjust the concentration of above-mentioned component, e.g., antiserum, secondary antibody, formaldehyde or crystal violet, to be optimum depending on the experimental condition.

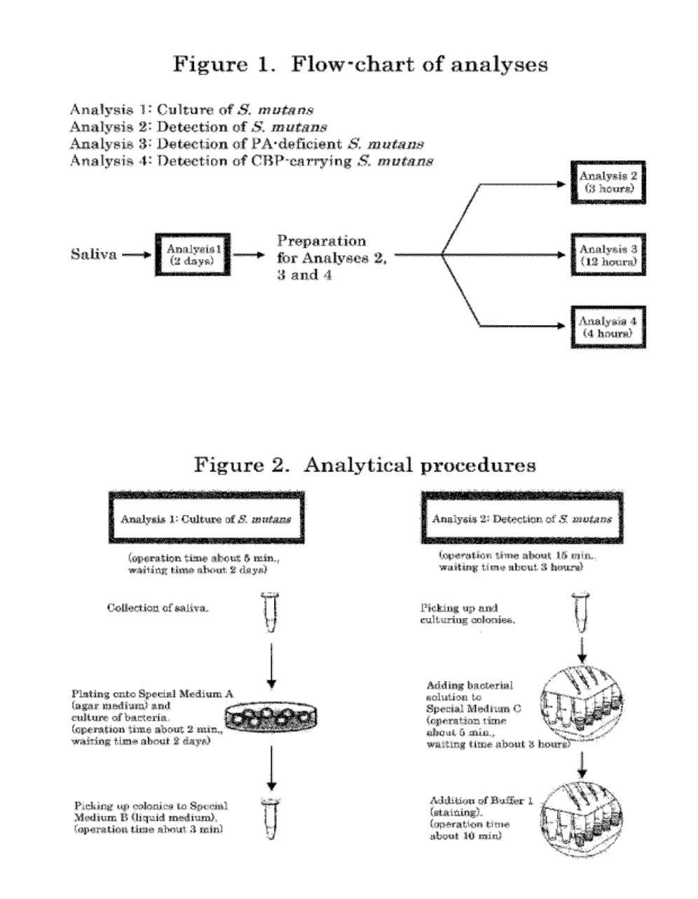

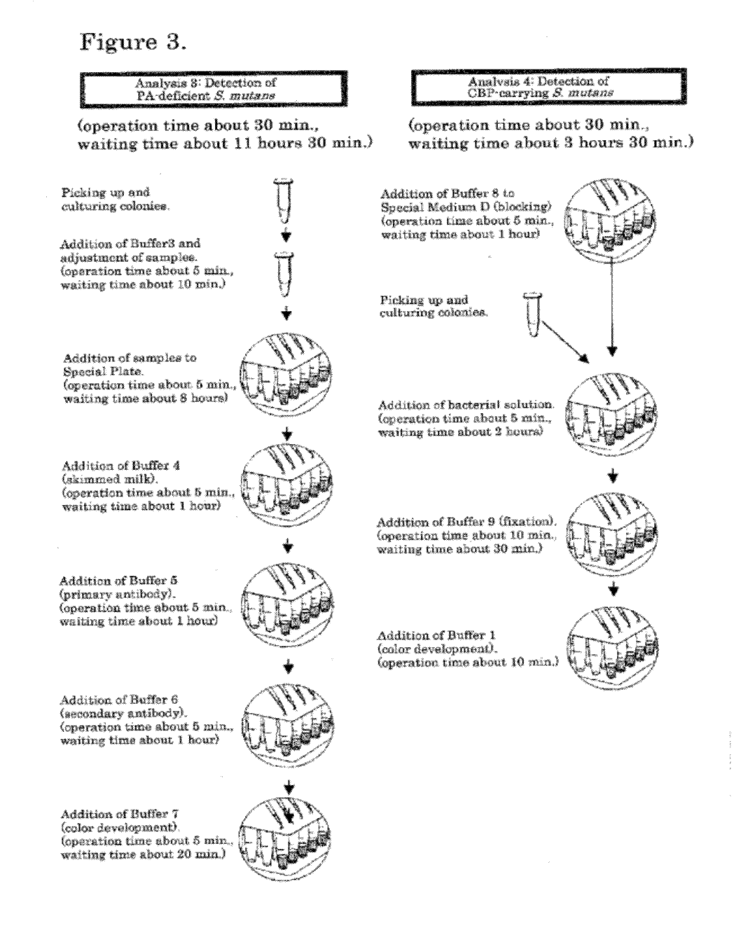

The method of the present invention for the detection of a hemorrhage-aggravating oral bacterium is carried out, specifically, for example in a scheme comprising following four steps as shown in FIGS. 1 to 3:

| | |
|---|---|
| Analysis 1. | Culturing of S. mutans |
| Analysis 2. | Detection of S. mutans |
| Analysis 3. | Detection of PA-deleted S. mutans |
| Analysis 4. | Detection of CBP-carrying S. mutans |

In Analysis 1, culturing of bacteria is carried out by following procedures using for example instruments and reagents in the aforementioned kit for culturing mutans streptococci.

The saliva of the subject is collected in a small amount using a spitz for collecting saliva. 10 μl of the saliva is taken from the spitz using a dropper, plated onto a S. mutans selection agar medium (e.g., above-mentioned Special Medium A), and cultured at 37° C. for 48 hours, preferably under an anaerobic condition. After culturing, the presence of bacterial colonies are grossly confirmed, colonies are picked up and added to a liquid medium (e.g., above-mentioned Special Medium B) and cultured for 37° C. for 18 hours, then used for the following Analysis 2, 3 and 4. Preferably, rough colonies are picked up, since S. mutans forms rough colonies, whereas S. sobrinus forms smooth colonies.

In Analysis 2, detection of S. mutans is carried out by following procedures using for example instruments and reagents in the aforementioned kit for detecting S. mutans.

10 μl of the bacterial solution cultured from the method of Analysis 1 is added to a medium (e.g., above-mentioned Special Medium C), incubated at 37° C. for 3 hours. The medium is washed with a wash buffer (e.g., above Wash Buffer A) for three times, then left still about 15 minutes with the last wash buffer. The wash buffer is removed, and again the medium is washed with Wash Buffer A for once, then a buffer containing a Gram-positive bacteria staining reagent (e.g., above Buffer 1) is added and left still for 1 minute. It is washed with the wash buffer for three times, and a buffer containing a mordanting agent (e.g., above Buffer 2) is added. If the color of the medium was changed, it is determined to be S. mutans-positive, if the color of the medium is unchanged, it is determined to be S. mutans-negative. A reagent in which a staining reagent and a mordanting agent are already combined may also be used.

In Analysis 3, detection of PA-deleted S. mutans is carried out by following procedures using for example instruments and reagents in the aforementioned kit for detecting PA-deleted S. mutans.

### (1) Sample Preparation

To the bacterial solution cultured by the method of Analysis 1 above a suitable buffer (e.g., above-mentioned Buffer 3) is added, which is then immersed in boiling water for 10 minutes, and frozen if it is to be stored.

### (2) Detection of PA-Deleted S. Mutans

1) The sample produced from (1) above is added to a plate, left still overnight at 4° C.
2) The plate is washed three times with a wash buffer (e.g., above Wash Buffer B), then skimmed milk (e.g., above Buffer 4) is added thereto, and left still at room temperature for 1 hour.
3) The plate is washed three times with the wash buffer, then a primary antibody (e.g., above Buffer 5) is added, reacted at room temperature for 1 hour.
4) The plate is washed three times with the wash buffer, then a labeled secondary antibody (e.g., above Buffer 6) is added, reacted at room temperature for 1 hour.
5) The plate is washed three times with the wash buffer, then a color-developing reagent (e.g., above Buffer 7) is added, and after appropriate time period, changes in the color of the solution are observed. When the color of the solution is changed, it is determined to be PA-positive, when the color of the solution is not changed, it is determined to be PA-negative.

In Analysis 4, detection of CBP-carrying S. mutans is carried out by following procedures using for example instruments and reagents in the aforementioned kit for detecting CBP-carrying S. mutans.
(1) The medium (e.g., above Special Medium D) is washed three times with a wash buffer (e.g., above Wash Buffer A), then albumin-containing buffer (e.g., above Buffer 8) is added, and left still at 37° C. for 1 hour.
(2) After washing three times with a wash buffer containing a surfactant (e.g., above

Wash Buffer C), bacterial solution cultured by'the method of Analysis 1 above is added, and incubated at 37° C. for 2 hours.
(3) After washing three times with the wash buffer (e.g., above Wash Buffer A), the fixative solution (e.g., above Buffer 9) is added and left still at room temperature for 30 minutes.
(4) After washing three times with the wash buffer, the Gram-positive bacteria staining reagent (e.g., above Buffer 1) is added and left still for 1 minute.
(5) After washing three times with Wash Buffer A, the mordanting agent (e.g., above Buffer 2) is added.

It is determined to be CBP-positive then the color of the solution is changed, and it is determined to be CBP-negative when the color of the solution is not changed.

In any of the detecting methods described above, the detection is possible if bacterial concentration is 1 CFU or more.

Moreover, a culture of e.g., S. sobrinus, S. sanguinis, S. oralis, S. gordonii, and S. salivarius may be used as a control to confirm in Analysis 1 that any bacterium other than S. mutans and S. sobrinus grows; in Analysis 3 that any bacterium other than PA-carrying S. mutans shows a positive reaction; and in Analysis 4 that any bacterium other than CBP-carrying S. mutans shows a positive reaction, respectively

A skilled person in the art may appropriately modify the method of the present invention according to its object. For example, for detecting PA-deleted S. mutans, a substrate to which a specific antibody for PA or CBP is attached may be contacted with a bacterial solution, washed to remove the bacteria which are not attached to the substrate, then only the bacterial cells that are attached to the substrate can be detected by the Gram-positive bacteria staining reagent. Alternatively, primers or probes for a PA or CBP-coding nucleic acid may be used to detect whether the cultured bacterium has the gene of PA or CBP.

In preferred embodiment of the present invention, S. mutans MT8148 strain may be used as a positive control for detection of a PA-deleted oral bacterium, and/or as a negative control for detection of a CBP-carrying oral bacterium. As a positive control for detection of a PA-deleted oral bacterium, depending on the detection method, an isolated PA protein, a nucleic acid or vector comprising a DNA encoding PA or its fragment, a cell transformed with said vector may also be used. As a negative control for detection of a CBP-carrying oral bacterium, CND strain, which is a TW295 strain in which CBP-encoding gene has been knocked out, and a Gram-positive bacterium that does not express CBP may also be used.

The present invention provides, in one embodiment, a hemostatic agent comprising an oral bacterial PA protein or a nucleic acid encoding the PA protein. When the subject has been infected with a PA-deficient, highly virulent bacterium, a hemostatic effect through the induction of platelet aggregation will be provided by supplying PA protein or expressing PA in the subject or bacterium.

Accordingly, the present invention also provides a use of an oral bacterial PA protein or a nucleic acid encoding the PA protein for the production of a hemostatic agent, as well as a method of hemostatic method comprising a step of administering an oral bacterial PA protein or a nucleic acid encoding the PA protein.

The present invention provides, in another embodiment, an inhibitor of platelet aggregation caused by a PA-expressing oral bacterium, the inhibitor comprising a substance that binds to an oral bacterial PA protein or a nucleic acid encoding the PA protein. When the subject has been infected with a PA-expressing oral bacterium, PA in the bacterial cell surface layer may be blocked by a substance that binds to PA protein, or the production of PA by the bacterial cell may be inhibited by a substance that inhibits the expression of PA protein, thereby inhibiting the platelet aggregation effect of the bacterium can be inhibited.

Accordingly, the present invention also provides a use of a substance that binds to an oral bacterial PA protein or a nucleic acid encoding the PA protein for the production of an inhibitor of platelet aggregation caused by a PA-expressing oral bacterium, as well as a method of inhibiting platelet aggregation caused by a PA-expressing oral bacterium comprising a step of administering a substance that binds to an oral bacterial PA protein or a nucleic acid encoding the PA protein.

The present invention provides, in another embodiment, an inhibitor of hemorrhage aggravation comprising a substance that binds to an oral bacterial CBP or a nucleic acid encoding the CBP protein. When the subject has been infected with a CBP-expressing hemorrhage-aggravating oral bacterium, using a substance that binds to CBP, e.g., a CBP-specific antibody, the CBP protein in the bacterial cell surface layer may be blocked and the binding of the bacterial cell to collagen-denuded site (i.e., the damaged site of vascular endothelia) may be inhibited, thereby treating or preventing hemorrhage aggravation. Alternatively, by using a nucleic acid encoding a substance that binds to CBP protein (e.g., an siRNA, antisense nucleic acid), CBP production by a bacterial cell can be inhibited, thereby inhibiting the binding of the bacterial cell to collagen-denuded site.

Accordingly, the present invention also provides a use of a substance that binds to an oral bacterial CBP or a nucleic acid encoding the CBP protein for the production of a hemorrhage aggravation inhibitor, as well as a method of inhibiting hemorrhage aggravation comprising a step of administering a substance that binds to an oral bacterial CBP or a nucleic acid encoding the CBP protein.

The present invention provides, in another embodiment, an agent for detecting collagen-denuded site in tissue comprising CBP of an oral bacterium. When connective tissue collagen is denuded due to vascular endothelia injury, the damaged site can be detected using the detecting agent of the present invention. Particularly, the detecting agent of the present invention allows noninvasive detection of the damaged site even if the hemorrhage site is in an area difficult to be detected, e.g., in head. Various labels may be added to the detecting agent for the convenience of detection. The label may be selected from any known labels, e.g., any radioisotopes, magnetic bodies, a substance that binds to the above-mentioned components (e.g., an antibody), biotin, fluorescent substances, fluorophores, chemiluminescent substances, elements that induce nuclear magnetic resonance (e.g., hydrogen, phosphorus, sodium and fluorine) and enzymes.

Accordingly, the present invention also provides a use of oral bacterial CBP for the production of an agent for detecting collagen-denuded site in tissue, as well as a method of detecting a collagen-denuded site in tissue comprising a step of administering an oral bacterial CBP.

Furthermore, the present invention provides, in another embodiment, a carrier for delivering a substance to the collagen-denuded site comprising an oral bacterial CBP. The hemostatic agent of the present invention or other drugs (e.g., an antibiotic or an anti-inflammatory agent) can be incorporated into the delivering carrier and administering it to an organism to target the hemostatic agent and the drugs to the damaged site, thereby expecting a damaged site-specific therapy. The carrier may be, for example, a liposome fused with a CBP protein or its collagen binding domain (CBD). To the carrier of the present invention, the hemostatic agent of the present invention or other drugs may be incorporated. Alternatively, the carrier of the present invention may be the CBP protein itself, and in this case, the therapeutic agent can directly be bound to the CBP protein or CBD.

The present invention provides, in another embodiment, a therapeutic agent for hemorrhage comprising an oral bacterial CBP and a hemostatic agent. The therapeutic agent for hemorrhage of the present invention is particularly useful in a subject having low platelet sensitivity to collagen. A subject having low platelet sensitivity to collagen includes a subject suffering such as aplastic anemia, acute leukemia, thrombocytopenic purpura, disseminated intravascular coagulation, thrombotic thrombocytopenic purpura, systemic lupus erythematosus, thrombasthenia or storage pool syndrome. Also, the therapeutic agent for hemorrhage of the present invention is particularly useful in a subject having a disease caused by a disorder of coagulation factor, such as hemophilia.

The CBP to be used for the carrier for substance delivery to the collagen-denuded site and therapeutic agent for hemorrhage of the present invention may be obtained, for example, by incorporating a nucleic acid construct comprising CBP gene into a suitable expression vector, and expressing CBP protein in the suitable host cell. Such techniques are well known in the art. For example, a plasmid, cosmid, phage, virus, YAC or BAC vector system comprising CBP gene can be incorporated into a host cell by various nucleic acid introducing method, e.g., calcium phosphate method, lipofection method, ultrasonic introduction method, electroporation method, particle gun method, microinjection method, liposome method (e.g., by cationic liposome), competent cell method or protoplast method to express CBP gene. CBP may also be the CBP-positive bacterium itself, or the CBP-containing component of the CBP-positive bacterium. Such component may be isolated by, for example, lysing and/or homogenizing CBP-positive bacteria and exposing to a substrate coated with Type I collagen. If the CBP-positive bacterium itself is to be used, said bacterium may be inactivated by a conventional method.

Moreover, the present invention relates to, in another embodiment, a prophylactic agent for hemorrhage aggravation comprising an agent for removing an oral bacterium.

According to the method of the present invention, in a case if a hemorrhage-aggravating oral bacterium has been detected, the hemorrhage-aggravating oral bacterium should be removed from the subject in order to alleviate the risk of hemorrhage aggravation and prevent it. As an oral bacterium-removing agent e.g., beta-lactam antibiotic may be used. A beta-lactam antibiotic includes, e.g., penicillin, methicillin, cephalosporin, cephamycin and carbapenems.

The hemostatic agent, platelet aggregation inhibitor, hemorrhage aggravation inhibitor, prophylactic agent for hemorrhage aggravation, therapeutic agent for hemorrhage, collagen-denuded site detecting agent and the carrier for substance delivery to the collagen-denuded site of the present invention may be administered by various routes encompasses oral and parenteral routes, such as, for example, oral, buccal, intravenous, intramuscular, subcutaneous, topical, rectal, intraarterial, intraportal, intraventricular, transmucosal, transdermal, intranasal, intraperitoneal, intrapulmonary and intrauterine routes, and may be formulated into a dosage form suitable for each administration route. Any known dosage form and method for formulation may be employed as appropriate (see, e.g., Watanabe et al., eds., 2003, HYOJUN YAKUZAIGAKU, Nanzando).

For example, formulations suitable for oral administration include, without limitation, a powder, granule, tablet, capsule, liquid, suspension, emulsion, gel and syrup. Formulations suitable for parenteral administration include injections such as an injectable solution, injectable suspension, injectable emulsion, and preparation-at-use injection. A formulation for parenteral administration may be in a form of aqueous or nonaqueous isotonic sterile solution or suspension. Specifically, for example, it may be formulated into a suitable unit dosage form, by combining appropriately with a pharmacologically acceptable carrier or medium such as, in specific, sterile water or physiological saline, vegetable oil, emulsifier, surfactant, stabilizing agent, excipient, vehicle, preservative or a binder. The amount of the effective ingredient in these formulations may be determined as appropriate so that a therapeutically effective amount can be provided to the subject in the defined dosage frequency.

Injectable aqueous solutions include, for example, a physiological saline, an isotonic solution comprising glucose and other adjuvant, e.g., D-sorbitol, D-mannose, D-mannitol and sodium, chloride. Appropriate solubilizing agent such as alcohol, specifically ethanol, a polyalcohol such as propyleneglycol, polyethyleneglycol, or a nonionic surfactant such as polysorbate 80 or HCO-50 may be used in combination.

Oily solutions includes e.g., a sesame oil and soy bean oil, which may be used in combination with a solubilizer such as benzyl benzoate or benzyl alcohol. Moreover, a buffering agent, e.g., a phosphate buffer, sodium acetate buffer, soothing agent, e.g., procaine hydrochloride, stabilizing agent, e.g., benzyl alcohol, phenol or antioxidant may be mixed. The injection prepared is usually filled in an appropriate container such as an ampoule, vial, tube, bottle or a pack.

Administration of hemostatic agent, platelet aggregation inhibitor, hemorrhage aggravation inhibitor, prophylactic agent for hemorrhage aggravation, therapeutic agent for hemorrhage, collagen-denuded site detecting agent and the carrier for substance delivery to the collagen-denuded site of the present invention into the body of subject may be via any of the above-mentioned routes, though, preferably, it is parenteral administration, more preferably topical or intravenous administration, particularly preferably intraportal or intratumoral administration. The frequency of dosage is preferably at once, though plurality of dosage may be used depending on the situation. The duration of dosage may be short, or may be sustained for a long time. More specifically, the composition of the present invention may be administered by injection or transdermally. The examples of administration by injection include but not limited to, e.g., by local injection, intravenous injection, intra-arterial injection, selective arterial infusion, portal vein injection, intramuscular injection, intraperitoneal injection, subcutaneous injection, intratumoral injection, intrathecal injection, intra-articular injection, intraventricular injection. An intravenous injection allows an administration in a manner of an ordinal blood transfusion, requiring neither a surgical operation to the subject nor local anesthesia, thus enabling alleviating the burden of both the subject and the operator. Moreover, it is advantageous that administration can be carried out elsewhere out of an operation room.

Furthermore, the present invention relates to, in one embodiment, a method of treating hemorrhage comprising administering an effective amount of the hemostatic agent, hemorrhage aggravation inhibitor, prophylactic agent for hemorrhage aggravation and/or therapeutic agent for hemorrhage described above to a subject. The present invention also relates to, in one embodiment, a method of treating a disease condition caused by platelet aggregation comprising administering an effective amount of the platelet aggregation inhibitor described above to a subject. Disease conditions caused by platelet aggregation include thrombosis and disseminated intravascular coagulation.

Moreover, the present invention relates to, in one embodiment, a method for diagnosing the site of hemorrhage comprising administering the collagen-denuded site detecting agent described above to a subject. Furthermore, the present invention relates to, a method of treating a disease associated with hemorrhage comprising administering an effective amount of the carrier for delivering a substance to the collagen-denuded site to a subject.

In the method of treatment or diagnosis of the present invention, the administration of the composition for treatment or diagnosis of the present invention to a subject may appropriately performed according to, for example, above-mentioned administration method. Also, a physician or veterinarian may appropriately modify the administration method described above to administrate the agent of the invention to a subject. Here, an effective amount is an amount of the hemostatic agent, hemorrhage aggravation inhibitor and/or therapeutic agent for hemorrhage described above that inhibits, alleviates or prevents the hemorrhage, or an amount of the platelet aggregation inhibitor that decreases the onset of, alleviates the symptoms or preventing the progress of a disease condition caused by platelet aggregation. It is preferably an amount that does not cause an adverse effect that exceeds the benefit by the administration. Such amount may be determined as appropriate by an in vitro examination using cultured cell, etc., or an examination in an animal model of such as a mouse, rat, dog or pig.

Specific amount of the composition for treatment or diagnosis of the present invention to be administered in the method of treatment or diagnosis of the present invention may be determined in consideration of various conditions associated with the subject in need of such treatment, e.g., the severity of the symptom, general health conditions of the subject, age, body weight and sexuality of the subject, diet, timing and frequency of administration, combination therapies, reactivity to the treatment, and the compliance to the treatment, etc., and thus may differ from the general effective amount, though, even in such cases, these methods are still encompassed within the scope of the present invention.

Routes of administration include various routes encompassing both oral and parenteral routes, e.g., oral, buccal, intravenous, intramuscular, subcutaneous, topical, intratumoral, rectal, intraarterial, intraportal, intraventricular (cardiac), transmucosal, transdermal, intranasal, intraperitoneal, intrathecal, intraarticular, intraventricular (brain), intrapulmonary and intrauterine routes.

The frequency of administration may vary depending on the characteristics of the composition to be used and the conditions of the subject as described above, though, for example, it may be plurality of times a day (namely, twice, three times, four times or five times or more a day), or once a day, once per several days (namely, e.g., every 2, 3, 4, 5, 6 or 7 days), once a week, once per several weeks (namely, e.g., every 2, 3 or 4 weeks).

Moreover, in the method of treating hemorrhage of the present invention, a drug other than the hemostatic agent, hemorrhage aggravation inhibitor and/or therapeutic agent for hemorrhage of the present invention which is effective for the treatment of a hemorrhage-associated disease described above may be used in combination. Also, in the method of treating a disease condition caused by platelet aggregation of the present invention, a drug other than the platelet aggregation inhibitor of the present invention which is effective for the treatment of a disease condition caused by platelet aggregation may be used in combination.

The term “subject” in the present invention means any living organism, preferably an animal, still more preferably a mammal, still more preferably a human individual.

### EXAMPLES

Hereinafter, the present invention is more specifically illustrated by way of examples, though the present invention is not to be limited by these examples.

### Materials and Methods

### Animal Experiments and Human Subject

All animal experiments in the present study has been carried out in accordance with the Guide for the Care and Use of Laboratory Animals published by US National Institutes of Health (NIH), and approved by the Institutional Animal Care and Use Committee of the Graduate School of Dentistry Osaka University and Hamamatsu University School of Medicine.

Study protocols using human samples has been approved by the ethics committee of the Graduate School of Dentistry Osaka University, Hamamatsu University School of Medicine and Suita Municipal Hospital (Suita City, Osaka, Japan). Before entry, all subjects were asked to sign to a consent form after the explanation about the protocols.

### S. Mutans Bacterial Strains and Culture Conditions

Major S. mutans strains used in the present study are shown in Table 1 (Reference 11, 21, 24, 25 and 29). Furthermore, 58 clinical S. mutans strains (strains isolated from blood: n=13, strains isolated from oral cavity: n=45) were used in the present study. All strains were cultured in Brain Heart Infusion (BHI) broth (Difco Laboratories, Detroit, Mich., USA), and erythromycin was added for the selection of the mutant strains. For each assay, bacterial cells were washed with PBS, and diluted to adjust the cell number.

TABLE 1
S. mutans used in this study.

| | | Protein | | |
|---|---|---|---|---|
| Strains | Serotypes | expressions | Features | References |
| TW295 | k | PA (−) Cnm (+) | Blood isolate from Japanese subject with | Fujiwara et |
| | | | bacteremia after tooth extraction | al. (2001) |
| TW295- | k | PA (−) Cnm (−) | Isogenic mutant with defect of Cnm | Nomura et |
| CND | | | constructed by TW295 | al. |
| TW871 | k | PA (+) Cnm (+) | Blood isolate from Japanese subject with | Fujiwara et |
| | | | Infective endocarditis complicated with | al. (2001) |
| | | | subarachnoid hemorrhage | |
| MT8148 | c | PA (+) Cnm (−) | Oral isolate from Japanese subject | Ooshima et |
| | | | | al. (1983) |
| MT8148- | c | PA (−) Cnm (−) | Isogenic mutant with defect of PA | Nakano et |
| PD | | | constructed by MT8148 | al. (2006) |
| SA53 | k | PA (−) Cnm (+) | Oral isolate from Finnish subject | Nakano et |
| | | | | al. (2008) |
| LJ32 | f | PA (−) Cnm (+) | Oral isolate from Japanese subject | Nakano et |
| | | | | al. (2008) |

### Collagen Binding Assay

The collagen binding properties of the mutant strain and parent strain were assessed by a modified version of the method of Reference 27 (Reference 22). The result for each strain was shown in a percentage relative to the binding of TW871.

### Platelet Aggregation Assay

Platelet aggregation assay were carried out using mouse whole blood by the impedance method with an aggregometer (Whole-blood aggregometer C540, Baxter Ltd., Tokyo, Japan). In brief, whole blood were taken from mice (ICR, male, 8 weeks old, body weight 35 to 40 g, CLEA Japan, Inc., Tokyo, Japan), and the mixture of the whole blood and various amount (10³, 10⁵ or 10⁷ CFU) of the bacterial cells were incubated at 37° C. for 5 minutes, then 4.0 pg collagen (native collagen fibril (Type I), Chrono-log Co., Havertown, Pa., USA) were added. The aggregation rate for each strain were calculated by the impedance (Ω) values in the presence or absence of the bacterial cells, and expressed as a percentage to that of the vehicle (where only collagen were added). Also, the platelet aggregation properties of 58 clinical strains and 3 MT8148 isogenic mutant strains were analyzed in the presence of 10⁷ bacterial cells.

### Assessment of Bacterial Cell Surface Charge (Zeta Potential)

The cell surface charge of the bacteria tested was measured using zeta potential analyzer (ELSZ-2, Otsuka Electronics, Co., Ltd., Hirakawa, Osaka, Japan). Said analyzer automatically calculates the zeta potential from the electrophoretic mobility using Smoluchowski equation. The bacterial cells cultured overnight were washed with PBS, adjusted to be 10⁷ CFU, loaded onto the analyzer, which automatically measured the zeta potential of the cells at five standard points. The results are shown as the mean values.

### Mouse Model of Brain Artery Injury

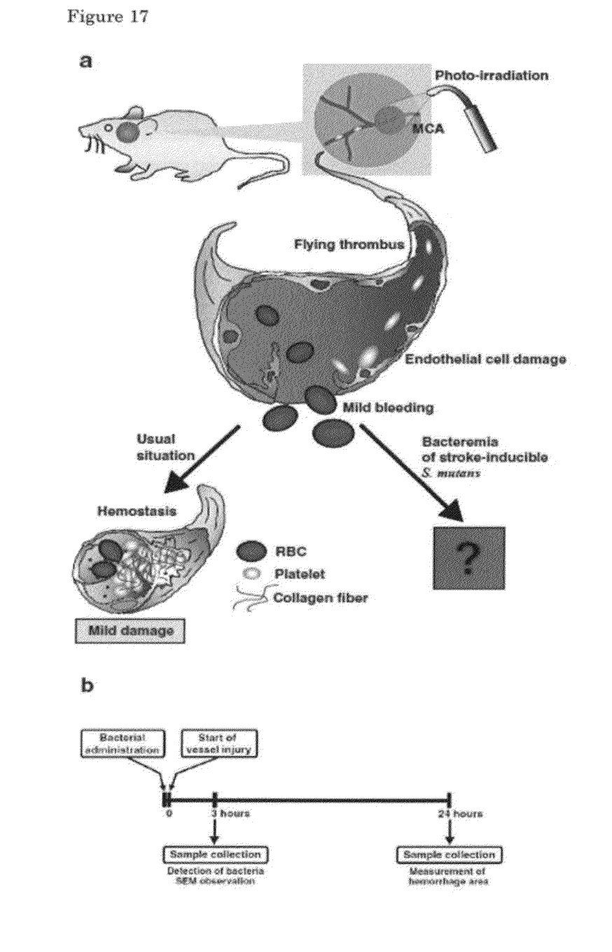

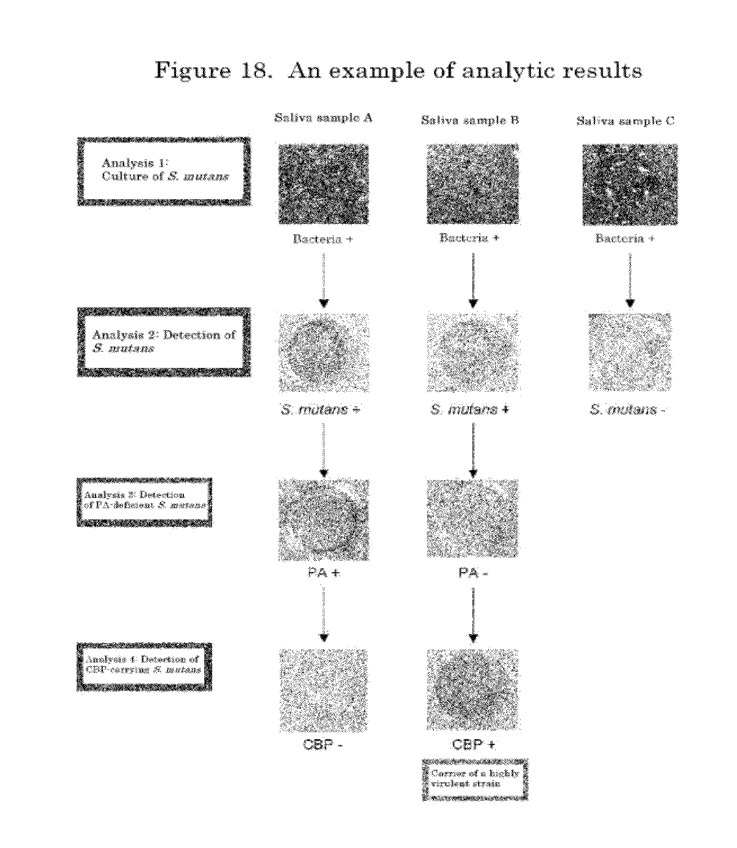

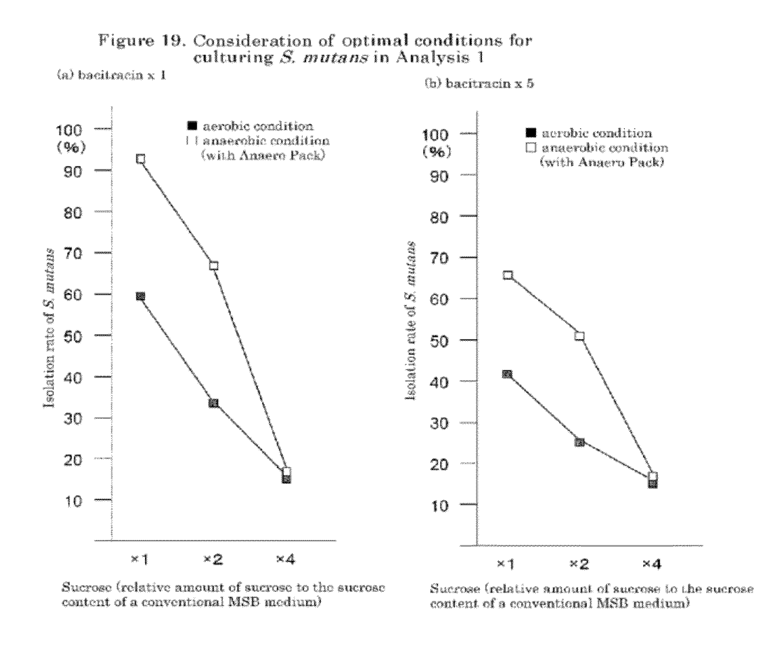

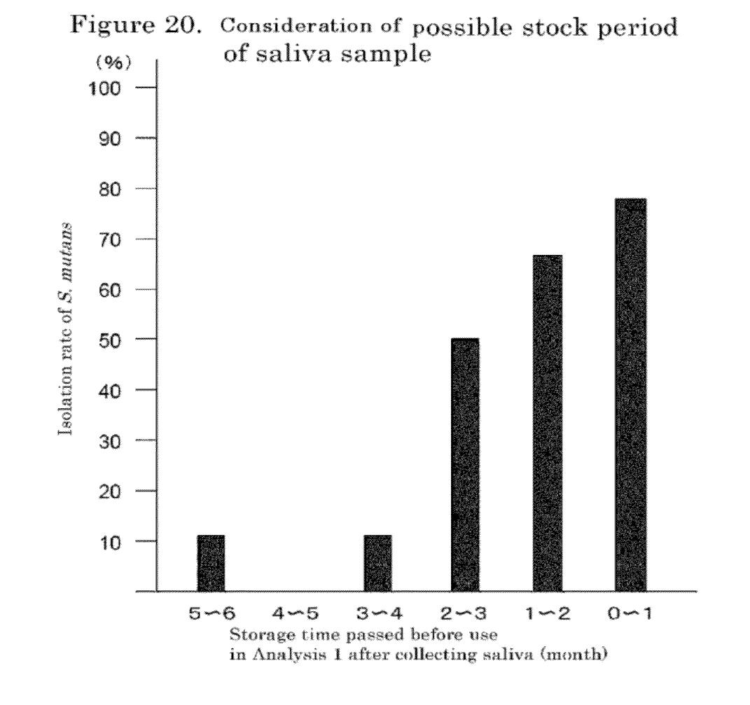

In mice, an injury was induced in vascular endothelial cells of the middle cerebral artery using a modified version of the already-described photochemical method (References 12, 28, FIG. 17). BALB/c mice (8 weeks old, male, body weight 20 to 30 g) were infected with the suspension of the test bacteria at 1×10⁷ CFU/mouse. Subsequently, Rose Bengal was administered, and a damage was given to the middle cerebral artery at one side via photosensitization for 10 minutes using a xenon lamp equipped with a heat absorption filter (0.04 W/cm², wavelength at 540 nm, Hamamatsu Photonics, Hamamatsu, Japan) and an optic fiber of 1.5 mm diameter installed onto the micromanipulator, thereby inducing the onset of a mild cerebral hemorrhage. The animals were euthanized 24 hours after the bacterial infection, and the resected brain tissue was sliced at certain intervals, and the area of total hemorrhage site from all brain slices was quantified in mm² unit by computer analysis according to the already-described method (References 12, 28, DP controller, Model DP70, OLYMPUS).

### Activation of Matrix-Metalloprotease (MMP-9)

Gelatin gel zymography was carried out by a modified version of already-described method (Reference 13). In brief, the tissue sample collected 24 hours after the administration of either tested bacteria or a vehicle was homogenized in a buffer containing 50 mM Tris-HCl, 150 mM NaCl, 1% Nonidet P-40, 0.1% SDS and 0.1% deoxycholic acid, pH7.4, supplemented with a protease inhibitor. Subsequently, the sample was separated using gelatin-zymo electrophoresis kit (Cosmo Bio., Tokyo, Japan).

### In Vivo Electron Microscopic Observation

Three hours after the induction of cerebral hemorrhage, the brain tissue was resected from the mouse, and the region of cerebral hemorrhage was observed with an electron microscope. In brief, the brain having a hemorrhage was fixed with 2% glutaraldehyde and dissected so that the section included a part of the obstacle, which was then fixed again with 1% osmium tetraoxide and dehydrated through an ethanol series. The sample was frozen, fractured into 2 to 4 pieces using a freeze-fracturing device filled with liquid nitrogen. The torn surface was perpendicular to the cerebral surface and included the hemorrhage site. Fractured samples were desiccated with a freeze-drying apparatus using t-butyl alcohol, then attached to the sample stage using a conductive paste so that the section came on top, and coated with osmium in order to confer conductance. The samples were observed with SEM.

Three-Dimensioned Computerized Tomography of Bacterial Cells Using Transmission electron Microscopy

Bacterial cell membranes were compared using three-dimensioned reconstructed images generated by a TEM CT (JEM 1220: JEOL Co., Tokyo). The TEM images of the bacterial cells were taken at ×150,000 magnification, at every 1° in a tilt range from −60° to +60°. The three-dimensioned reconstructed CT images were generated using Radon transform software. These CT images can be displayed in any direction.

### Detection of Bacteria in a Tissue Sample

Detection of bacterial infection in several organs was carried out as follows using PCR. Total DNA was extracted from resected tissues such as the damaged and undamaged hemispheres of the brain, lung, liver and intestine, and examined by PCR method using S. mutans-specific primers (Reference 9) below.

S. mutans-specific primers:
Forward:
(SEQ ID NO.: 11)
5′-GGC ACC ACA ACA TTG GGA AGC TCA GTT-3′
Reverse:
(SEQ ID NO.: 12)
5′-GGA ATG GCC GCTAAG TCAACA GGA T-3′

The detection limit of bacteria was from 5 to 50 cells in each sample. In order to confirm the presence of the viable cells in tissue, each tissue resected was compressed in PBS, then the stock and diluted solutions were streak-cultured on a bacitracin (100 units/ml; Sigma-Aldrich, St. Louis, Mo. USA)-containing Mitis-Salivariusagar plate (Difco) which is an agar plate for selective culture.

### Production of CBP Gene Knockout Strain (CND Strain): TW295CD

TW295 strain cnm gene fragment was amplified using following primers designed based on the full length sequence of cnm gene encoding CBP of TW295 strain (SEQ ID NO. 4: DDBJ Accession No. AB469913)

Primers for amplification of cnm:
(SEQ ID NO.: 13)
cnm1F 5′-GAC AAA GAA ATG AAA GAT GT-3′
(SEQ ID NO.: 14)
cnm1R 5′-GCA AAG ACT CTT GTC CCT GC-3′

Amplified fragment was incorporated into pGEM-T Easy vector (Promega, Madison, Wis., USA) to generate the plasmid pTN11. pTN11 was treated with the restriction enzyme BsmI to digest the middle part of the open reading flame of cnm and generated the plasmid pTN12, in which an erythromycin-resistant gene fragment obtained from the plasmid pKN100 was incorporated. pTN12 was disassembled into single strands using the restriction enzyme PstI, and homologously recombined into TW295 strain by a chemical procedures using horse serum. The screening of a strain having an erythromycin resistant gene in the middle part of cnm gene (CND strain) was carried out using an erythromycin-containing S. mutans-selection medium. Generated strain was confirmed by Southern hybridization and measurement of collagen binding ability.

### Production of PA Gene Knockout Strain (PD Strain): MT8148PD

According to the method described in Nakano et al. Microbes Infect. 2006 8(1)114-21, PD strain was generated and confirmed by a similar method as the CND strain above using primers based on the full sequence of pac gene encoding PA of MT8148 strain (SEQ ID NO. 2: DDBJ Accession No. X14490).

Primers for the amplification of pac:
pacF
(SEQ ID NO.: 15)
5′-GCG CGC ATG CTT TAT TCA GAT TTG GAG GAT-3′
pac-R
(SEQ ID NO.: 16)
5′-GCG AAA GCG CAT GCT GTG ATT TAT CGC TTC-3′

### Statistical Analysis

Statistical Analysis was performed using Prism 4 software (GraphPad Software Inc., San Diego, Calif., USA). Fisher's PLSD, Student's t-test, regression analysis and ANOVA was performed. The result was considered significant if p<0.05.

### Statistics of the Frequency of Carrying S. Mutans Bacterial Surface Protein

For 170 strains of S. mutans isolated from 170 child patients who consulted to Osaka University Graduate School of Dentistry, Department of Pediatric Dentistry from 2002 to 2003, frequencies of carrying PA and CBP. Furthermore, the malignancy of hemorrhage aggravation in representative bacterial strains was determined in mouse cerebral hemorrhage model.

### Results

### Example 1

### Examination of the Virulence of Streptococcus Mutans in Mouse Cerebral Hemorrhage Model

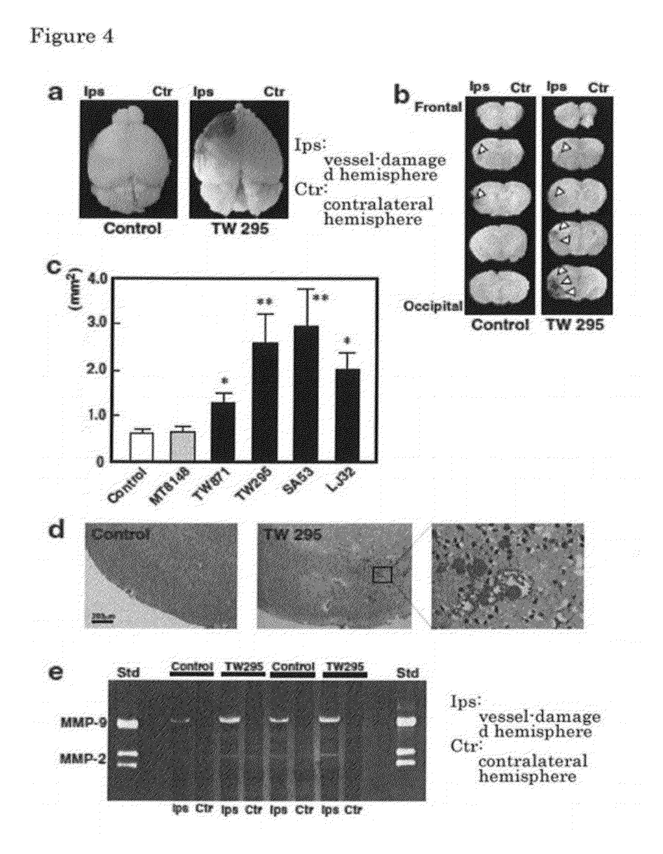

At 24 hours after the onset of cerebral hemorrhage, in the control group which had not been given the bacteria at all, a mild cerebral hemorrhage was confirmed in the downstream of middle cerebral artery in the vessel-damaged hemisphere (FIGS. 4a [symbol] 4b). This cerebral hemorrhage induces cerebral infarction (Reference 12). In the group which had been infected with MT8148 strain, no exacerbation of cerebral hemorrhage was confirmed as compared to vehicle control (FIG. 4c). On the other hand, in the group which had been given a serotype k TW871 strain isolated from a patient with subarachnoid hemorrhage, a dramatic exacerbation of cerebral hemorrhage was confirmed (FIG. 4c). Similarly, in groups of mice which had been infected with other serotype k strains TW295, SA53 or LJ32 strain, a significant increase in hemorrhage area was observed as compared to the control group and the group infected with MT8148 strain (FIG. 4). TW295 and SA53 strain induced the maximum increase in cerebral hemorrhage area. However, administration of TW295 strain itself did not cause any changes in blood pressure, heart rate and cerebral blood flow (Tables 2 and 3). Microscopic observation confirmed an evident hemorrhage 3 hours after the induction of cerebral hemorrhage in a mouse infected with TW295 strain (FIG. 4d).

TABLE 2
Parameters of circulation.

| | | Time after administration (min) | | | | | |
|---|---|---|---|---|---|---|---|
| Parameters | Pre | 5 | 10 | 15 | 20 | 30 | 40 |
| Systolic blood pressure (mmHg) | | | | | | | |
| Control | 83.0 ± 2.5 | 77.3 ± 3.7 | 78.0 ± 2.0 | 79.7 ± 2.8 | 80.3 ± 2.6 | 79.0 ± 1.5 | 78.0 ± 2.5 |
| TW295 | 75.5 ± 1.3 | 74.3 ± 4.8 | 76.0 ± 1.0 | 74.3 ± 1.4 | 73.3 ± 1.4 | 74.5 ± 0.9 | 73.8 ± 0.6 |
| Diastolic blood pressure (mmHg) | | | | | | | |
| Control | 73.3 ± 2.4 | 68.7 ± 3.5 | 68.7 ± 1.9 | 70.7 ± 2.8 | 70.0 ± 2.3 | 68.7 ± 1.9 | 66.7 ± 2.2 |
| TW295 | 68.0 ± 1.4 | 66.3 ± 4.1 | 66.8 ± 1.1 | 65.3 ± 1.6 | 64.8 ± 1.3 | 65.5 ± 0.3 | 64.5 ± 0.3 |
| Mean blood pressure (mmHg) | | | | | | | |
| Control | 76.0 ± 2.1 | 71.7 ± 3.4 | 72.7 ± 2.4 | 72.7 ± 2.3 | 73.3 ± 1.8 | 72.3 ± 1.5 | 71.0 ± 2.1 |
| TW295 | 69.0 ± 1.0 | 68.8 ± 4.7 | 70.0 ± 0.4 | 68.5 ± 1.2 | 67.3 ± 1.3 | 68.6 ± 0.9 | 67.3 ± 1.1 |
| Heart rate (beats/min) | | | | | | | |
| Control | 428.3 ± 30.6 | 403.0 ± 33.8 | 409.3 ± 32.7 | 409.3 ± 29.8 | 407.7 ± 29.8 | 403.0 ± 28.0 | 399.3 ± 28.1 |
| TW295 | 439.0 ± 14.5 | 426.0 ± 9.3 | 425.5 ± 10.9 | 424.8 ± 11.6 | 421.3 ± 11.3 | 414.5 ± 12.4 | 411.5 ± 14.5 |

Mean ± SEM (n = 3-4).

TABLE 3
Cerebral blood flow.

| Parameters | Control | TW295 |
|---|---|---|
| Occlusion time (sec) | 396.7 ± 126.7 | 469.0 ± 101.8 |
| Total flow time (sec) | 1612.0 ± 644.9 | 1749.3 ± 287.6 |

Mean ± SEM (n = 3-4).

Moreover, the effect of TW295 strain on activation of matrix-metalloprotease (MMP)-9 was investigated. Destruction of vascular obstacle by activated MMP-9 is an important amplifying route that causes further hemorrhage (References 12, 13). As shown in FIG. 4e, in the vessel-damaged hemisphere of the damaged mouse brain, an activation of MMP-9 was confirmed. The administration of TW295 strain stimulated further activation of MMP-9 in the vessel-damaged hemisphere as compared to the control. However, it should be noted that the administration of TW295 strain in a mouse having no cerebral artery injury did not cause any damage to the tissue of the mouse even at 24 hours after the onset of cerebral hemorrhage (FIGS. 4a and 4b). These results suggest that both a cerebrovascular event and the presence of a serotype k bacterium are necessary for aggravation of cerebral hemorrhage.

### Example 2

### Examination of the Relation Between Collagen Binding Activity and Cerebral Hemorrhage

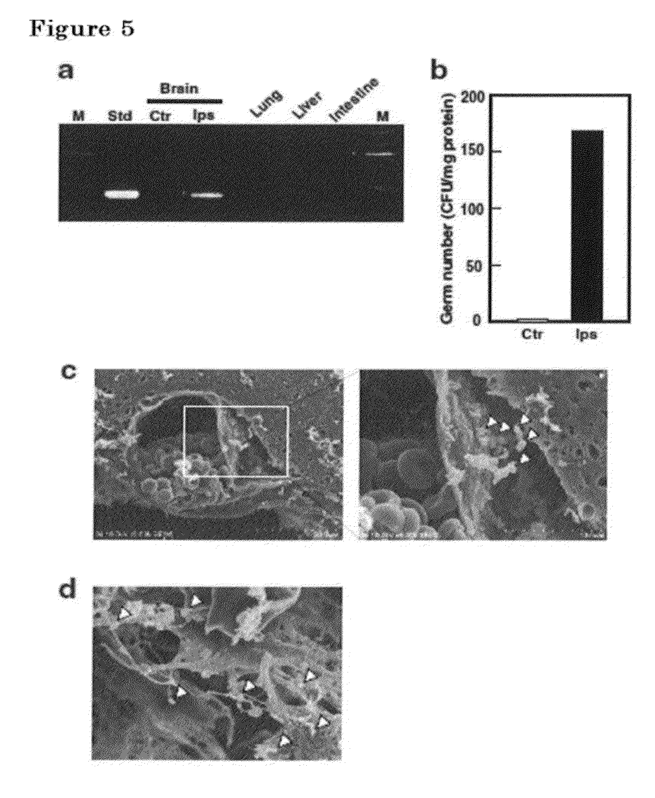

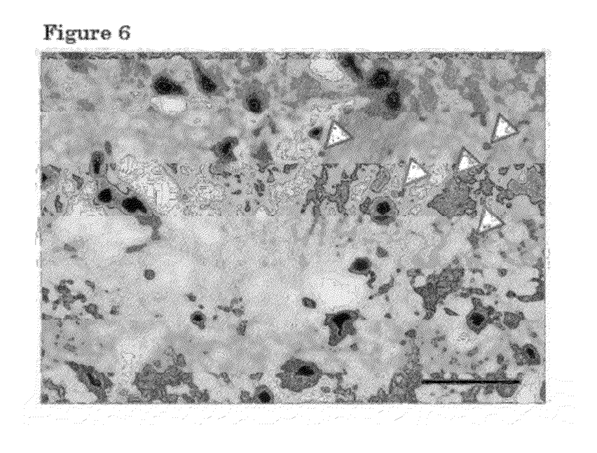

In order to testify the hypothesis that the administered bacteria are localized specifically to the damaged site, the localization of S. mutans in the damaged tissue after bacterial administration was investigated. The transfer of the bacteria to each organ was examined by PCR method, and transfer of the administered TW295 strain was observed only the ipsilateral hemisphere of the vascular injury, but not in other parts of the brain or in other organs (FIGS. 5a and 5b). Furthermore, in vivo electron microscopic observation (FIG. 5c) and optical microscopy observation (FIG. 6) confirmed the localization of the bacterial cells in the vessels in the damaged hemisphere in which vascular endothelia had been damaged and collagen fibers had been denuded. Moreover, attachment of the bacterial cells to collagen fibers in the damaged vessels (FIG. 5d). These results suggest that the administered bacteria specifically interact in vivo with the damaged vessels, especially via denuded collagen fibers.

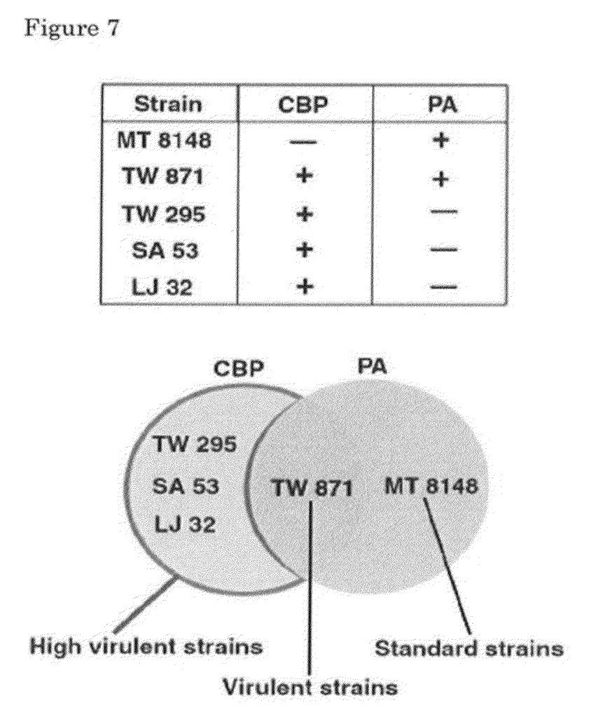

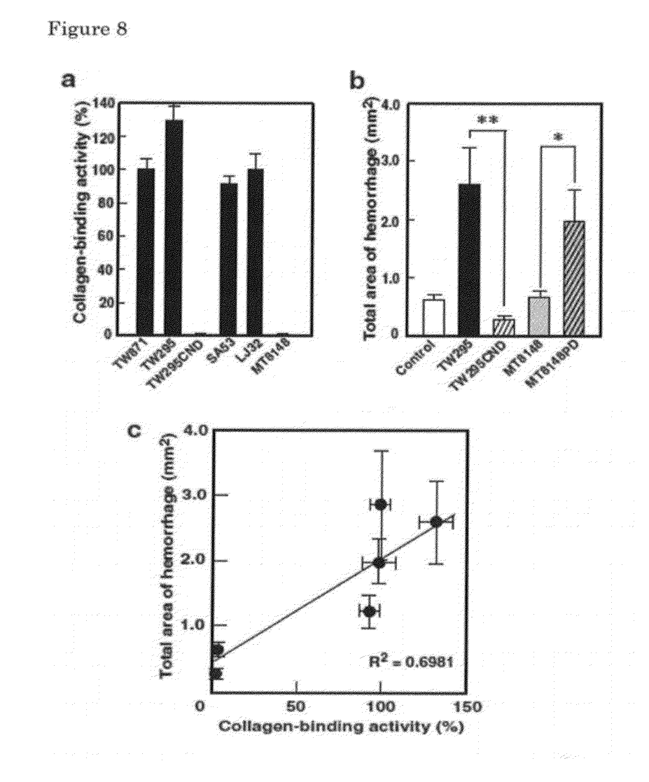

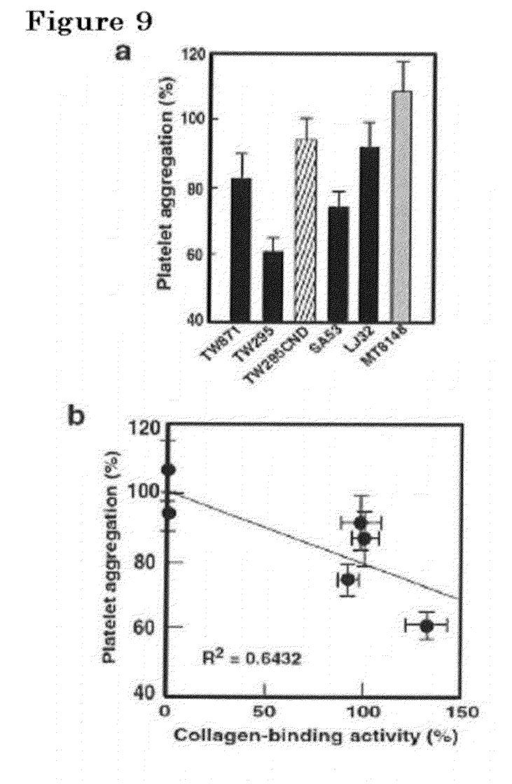

Accordingly, the inventors focused on the direct interaction of serotype k S. mutans and collagen fibers. It has been known that denuded collagen fibers are present in the vascular surface of the vessel damaged by the disruption of endothelial cells, and that the interaction of the collagen fibers and platelets is important for platelet aggregation. Recently, a cell surface collagen binding protein of 120 kDa on (CBP, also known as collagen binding adhisin and Cnm) has been identified in S. mutans, and its coding gene (cnm) has been cloned and its sequence has been disclosed (Reference 14). Among S. mutans clinical strain, about 10% are carrying CBP, and their distribution is dominant in serotype k or f strain (Reference 15 and 16). Interestingly, all of the highly virulent strain observed in the cases of human cerebral hemorrhage described hereinbelow (TW871, TW295, SA53 and LJ32, see, FIG. 4c) have this surface protein (FIG. 7). In fact, it was shown that the collagen binding activity in vitro of the highly virulent strain was dramatically higher than MT8148 strain (FIG. 8a). Furthermore, it was evidenced that in vitro treatment of blood with highly virulent bacterium decreases the level of platelet aggregation as compared to the case when MT8148 strain is used (FIG. 9a).

The inventors generated a mutant strain (TW295CND, Table 1) that is deficient in expression of collagen binding adhisin, from TW295 strain. Suppression of platelet aggregation observed in TW295 strain was completely recovered in TW295CND strain (FIG. 9a). These results indicate collagen binding protein is necessary for collagen binding activity and platelet aggregation inhibitory activity of TW295 strain.

Subsequently, the inventors administered TW295CND strain to a mouse cerebral hemorrhage model. As shown in FIG. 8b, the area of cerebral hemorrhage in the TW295CND-administered mouse was dramatically lower than those in the TW295 strain-administered mice. There was an evident interaction between collagen binding activity and hemorrhage area (FIG. 8c), as well as between collagen binding activity and platelet aggregation inhibitory ability (FIG. 9b), respectively. These results indicate that collagen binding protein in serotype k S. mutans is a major cause of the high virulence of the bacterium of this serotype in cerebral hemorrhage. Also, the PA-knockout mutant strain (MT8148PD) derived from the S. mutans standard strain MT8148 exacerbated cerebral hemorrhage as compared to the control (FIG. 8b). This result indicates that the deficiency in PA expression is involved in aggravation of cerebral hemorrhage by a highly virulent strain that expresses collagen binding protein.

### 3. Inhibition of Platelet Aggregation by Serotype k S Mutans

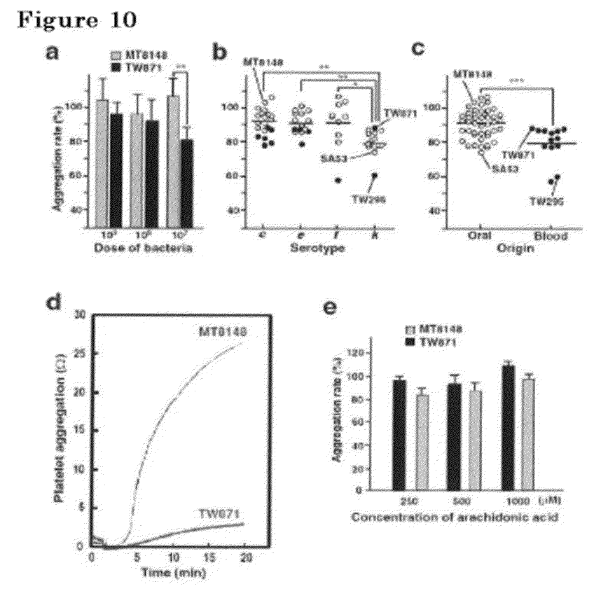

Platelet aggregation is the most important step to hemostasis after a vessel injury. Effects of S. mutans of various serotypes on platelet aggregation induced by collagen were examined using mouse whole blood. The standard strain MT8148 did not show any platelet aggregation inhibitory effect in whole blood as compared to the vehicle control (FIG. 10a). On the contrary, serotype k TW871 strain showed a significant inhibition of platelet aggregation when 10⁷ cells were added to the whole blood (FIG. 10a).

Also, the effects of clinically isolated 58 other S. mutans strains on platelet aggregation were investigated. The platelet aggregation rate in the presence of a serotype k strain was significantly lower than other serotypes (p<0.05; FIG. 10b). Among these, TW295 strain showed the most potent platelet aggregation inhibition. Interestingly, the platelet aggregation rate in the presence of a blood-isolated strain was significantly lower than an oral cavity-isolated strain (p<0.001; FIG. 10c). A similar result was observed when TW871 strain was added to a platelet aggregation using platelet-rich plasma. Moreover, it was observed that the duration of hemorrhage tends to be longer after administrating TW295 strain to a mouse (data not shown).

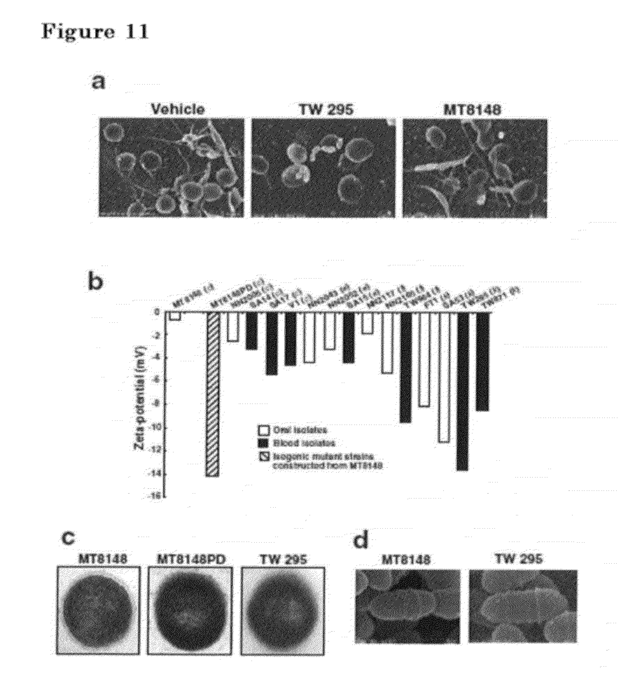

However, arachidonic acid-induced platelet aggregation was not inhibited by administration of TW295 strain (FIG. 10e). The inventors made a hypothesis that TW295 strain inhibits collagen thereby inhibiting platelet aggregation. As shown in the scanning electron microscope (SEM) images in FIG. 11a, in the platelet fraction collected after the stimulation by collagen, an interaction between platelets and collagen which results in platelet activation was observed (left panel, vehicle). In the vehicle control group, morphological changes associated with platelet aggregation such as pseudopodia and platelet adhesion were observed. Addition of MT8148 strain did not show any effects on the interaction between platelets and collagen as compared to the control (FIG. 11a, right panel). On the contrary, it was shown that addition of TW295 strain clearly suppressed the interaction between platelets and collagen, and resulted in the inhibition of platelet activation (FIG. 11a, middle panel). These results clearly indicate that the attachment of TW295 to collagen inhibits the interaction between platelets and collagen, thereby inhibiting the aggregation.

### 4. Assessment of Bacterial Surface Ionic Charge

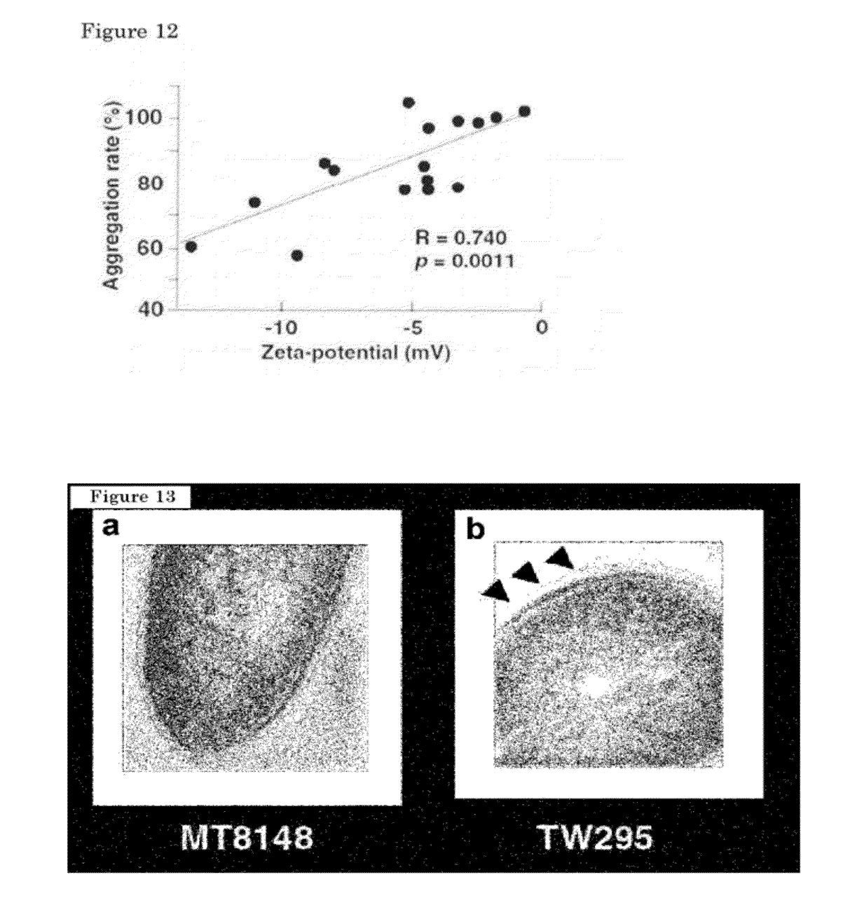

The ionic charge of the platelet surface is an important factor that induces an interaction with the denuded collagen fibers of the damaged vessel. The anionicity of the platelet surface provides an interaction with the cation charge of collagen (References 17 to 19). Accordingly, the ionic charge of bacterial cell surface which may influence the interaction with collagen was measured. The mean value of the zeta potential (which is used as an index of the cell surface ionic charge) of MT8148 cells lysed in physiological saline was −0.75 mV, which is almost nonionic (FIG. 11b). On the contrary, the zeta potential of serotype k strains such as TW295 strain and TW871 strain was −13.51 mV and −8.42 mV, respectively, showing much lower values than that of MT8148 strain, indicating that the cell surface condition of a serotype k strain is anionic (FIG. 11b). The regression analysis between the zeta potential value and the platelet aggregation rate indicated a significant positive correlation (FIG. 12).

Studies have been done in order to elucidate the role of S. mutans surface protein antigen as a virulence factor of dental caries, and a 190 kDa protein antigen (PA) has been known to be relevant to the initial attachment to dental surface (Reference 20). It has been shown that a PA-knockout strain has a decreased antigenicity as compared to a strain with a normal expression of PA, and thus maintains a prolonged duration of bacteremia (Reference 21). The cell surface condition of the PA-knockout isogenic mutant strain generated from

MT8148 (MT8148PD, Reference 21) was measured. The mean value of the zeta potential of MT8148PD was much lower than that of MT8148 strain (FIG. 11b). The presence or absence of these molecules is considered to be involved in the determination of the surface ionic charge of the serotype k S. mutans.

The transmission electron microscopy observation (TEM, FIG. 11c) and SEM observation (FIG. 11d) showed a remarkable difference in the cell surface between MT8148 strain and a serotype k strain such as TW295 strain. The PA-knockout strain generated from MT8148 strain showed a quite similar surface condition to that of TW295 strain (FIG. 11c). Bacterial membrane structure observed by TEM using ultra-thin sections may change depending on the direction of observation. In addition, it is necessary to consider the thickness of the section in ultrastructure observation. Accordingly, the bacterial membranes were compared using three-dimensional images reconstructed from computerized tomography (CT) of TEM. The peptidoglycan layer of MT8148 was observed as a transparent and smooth layer in a three-dimensional TEM image (FIG. 13a), whereas the peptidoglycan layer of TW295 was opaque and its boundary was obscure (FIG. 13b). These results clearly indicate that the bacterial surface containing the peptidoglycan layer greatly differ depending on the deficiency or the presence, and this difference results in the condition of the ionically charged surface of serotype k S. mutans.

5. S. Mutans Strain Isolated from Human Stroke Patients

In order to prove the hypothesis that the infection of CBP gene-expressing S. mutans is a risk factor of stroke, the frequency of the occurrence of S. mutans carrying the collagen binding protein among stroke patients using oral cavity samples. The results are shown in Table 4.

TABLE 4
Detection frequency of CBP-carrying S. mutans in stroke patients.

| No. | Age | Gender | Diagnosis | Major Complications | S. mutans | cnm |
|---|---|---|---|---|---|---|
| 1 | 64 | M | Cerebral Hemorrhage | Hypertension, Hyperlipidemia | + | ND |
| 2 | 75 | M | Cerebral Hemorrhage | Hypertension, Angina | + | + |
| 3 | 58 | M | Infarction | Stomach cancer | ND | ND |
| 4 | 84 | M | Infarction | Anemia, Renal Failure | + | + |
| 5 | 67 | M | Cerebral Hemorrhage | Cerebral aneurysm | ND | ND |
| 6 | 83 | M | Infarction | Hypertension | ND | ND |
| 7 | 75 | M | Cerebral Hemorrhage | Hypertension | ND | ND |
| 8 | 58 | M | Infarction | Hypertension, Diabetes | + | ND |
| 9 | 63 | F | Infarction | Hyperlipidemia | + | ND |
| 10 | 80 | F | Infarction | Hypertension, Diabetes | + | ND |
| 11 | 81 | M | subarachnoid hemorrhage | | ND | ND |
| 12 | 76 | F | Infarction | Hypertension | + | + |
| 13 | 70 | F | Infarction | Hypertension, Diabetes | + | ND |
| 14 | 51 | F | Cerebral Hemorrhage | Hypertension, Hyperlipidemia | ND | ND |
| 15 | 67 | M | Cerebral Hemorrhage | Hypertension, Hyperlipidemia | + | + |
| 16 | 62 | M | Infarction | Hypertension | + | ND |
| 17 | 49 | M | Cerebral Hemorrhage | | + | + |

ND: not detected because of lower than detection limit (10 CFU/ml).

Among 17 cases of stroke patients, S. mutans was isolated from the patients in 11 cases. 5 cases among those were infected with CBP gene-expressing S. mutans (5/11, 45.5%, Table 4). This is much higher than the frequency of detecting collagen binding protein-carrying S. mutans in healthy subjects (10%). These results suggest that the infection with CBP gene-expressing S. mutans is likely to be a risk factor of stroke.

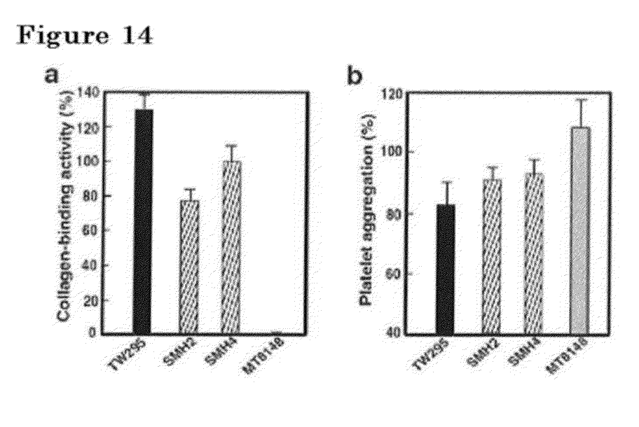

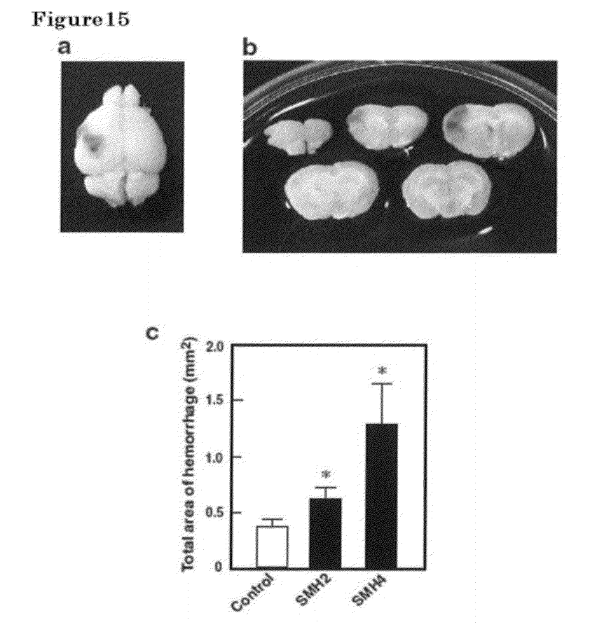

Furthermore, the virulence of isolated CBP gene-expressing S. mutans was examined in mice. Among the CBP-expressing S. mutans strains isolated from stroke patients, two strains (SMH4 and SMH6, FIGS. 14a and 14b) in which both collagen binding activity and platelet aggregation inhibitory activity are higher as compared to those of wild-type MT8148 strain were investigated in a mouse cerebral hemorrhage model. In a mouse to which either SMH4 or SMH6 strain had been administrated, a dramatic aggravation of cerebral hemorrhage was observed as compared to the control (wild-type MT8148 strain) (FIGS. 15a, 15b and 15c). These results clearly indicate that the S. mutans strain isolated from a stroke patient is a risk factor that causes stroke.

### 6. Correlation Between the Frequency of Occurrence of PA and CBP-Carrying Strain and the Malignancy of Hemorrhage Aggravation.

Table 5 summarizes the results of the investigation of the frequency of carrying bacterial surface protein by S. mutans for 170 strains of S. mutans isolated from 170 child patients. Malignancy was estimated from the area of hemorrhage region caused by each bacterial strain in the mouse cerebral hemorrhage model.

TABLE 5
Expression frequency of cell surface proteins for S. mutans.

| Frequency | | | | |
|---|---|---|---|---|
| in the oral cavity | S. mutans | PA | CBP | malignancy |
| 1.8% | + | − | + | 100% |
| 1.2% | + | − | − | 50-70% |
| 8.2% | + | + | + | 40-60% |
| 88.8% | + | + | − | 0 |

Strains that do not express PA shared 3% of the overall, while strains that do not carry CBP occupy 90% of the total. Malignancy in cerebral hemorrhage was determined to be the highest in 1.8% of strains that do not carry PA and that carries CBP from the area of hemorrhage region caused by each bacterial strain, and which was defined as 100% malignancy. According to this definition, the malignancy of the strains that do not express PA and that do not carry CBP (frequency=about 1.2%) and the strains that express PA and that carry CBP (frequency=about 8.2%) were determined about 50 to 70% and about 40 to 60%, respectively. This result agrees to the experimental results using PA and CBP gene knockout strains described above.

### Discussion.

In the present study, it is first shown that a CBP-expressing and/or PA-deficient S. mutans is potential risk factor of a disease associated with hemorrhage, especially hemorrhagic stroke.

In the present study, an aggravation of cerebral hemorrhage by serotype S. mutans strain was confirmed. Furthermore, since infectious bacteria were detected only in the vessel-damaged hemisphere but not in the contralateral hemisphere, it was shown that the interaction between the serotype k S. mutans and the damaged vessel is an important event in the onset of cerebral hemorrhage. These strains show the expression of the collagen binding protein (CBP) and/or the deficiency in the protein antigen (PA) as a common protein expression pattern, which are shown to be important in aggravation of cerebral hemorrhage (FIG. 7). The hypothesis by the inventors that the collagen binding protein of a serotype k S. mutans is involved in the onset of cerebral hemorrhage is supported by the present result that the collagen binding protein-deficient mutant strain TW295CND did not induce an aggravation of cerebral hemorrhage. The highly virulent strains TW295, TW871, SA53 and LJ32 all express on their surface the collagen binding protein, and have a potent collagen binding property. Accordingly, due to the accumulation of a bacterial strain having a potent collagen binding property to the denuded collagen layer, activating MMP-9 and inhibiting platelet aggregation, further bleeding is brought about. Therefore, a strain having the collagen binding protein should be considered as a highly virulent strain of cerebral hemorrhage. In fact, an in vivo SEM imaging of damaged vessel in a mouse brain demonstrated that although there were infectious bacteria, no platelet aggregation had been occurred.

Another potential virulent factor of cerebral hemorrhage is the deficiency in protein antigen (PA) expression. The highly virulent strains TW295, SA53 and LJ32 all were shown to be deficient in PA expression. On the other hand, TW871 expresses PA antigen (FIG. 7 and Table 1), and therefore the cerebral hemorrhage area in TW871 strain-treated mouse was much smaller than the cerebral hemorrhage area in mice treated with other highly virulent strains (FIG. 4c). Moreover, PA-knockout strain derived from the S. mutans standard strain MT8148 (MT8148PD) showed cerebral hemorrhage aggravation as compared to the control (FIG. 8b). Furthermore, the platelet aggregation rate was significantly lower in strains showing no PA expression as compared to in strains showing PA expression (data not shown). These results indicate the relevance of PA deficiency to cerebral hemorrhage aggravation by a highly virulent strain expressing the collagen binding protein.

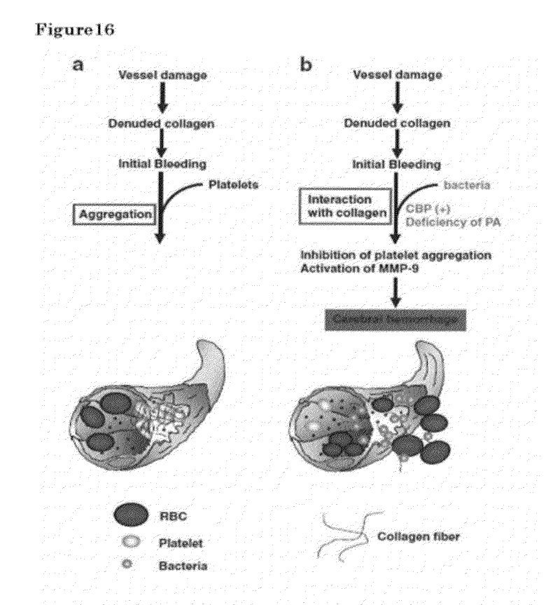

In general, collagen is cationic under physiological conditions, and therefore the ionic properties of bacterial surface are considered to be important in their interaction with denuded collagen fibers. In fact, PA-deficient isogenic mutant shows the lowest zeta potential value, and other PA-knockout strains also tend to have a low zeta potential value. This indicates that PA influences zeta potential value. Because there was a positive correlation between the zeta potential value and collagen-induced platelet aggregation rate, a strain having a low zeta potential value can also be categorized as a highly virulent strain. From these results, it can be considered that a strain expressing S. mutans collagen binding protein possesses a high affinity to denuded collagen fibers, and a low level expression of PA in S. mutans inclines the cell surface condition to be anionic, which further increases the affinity with cationic collagen fibers. The synergic effect of the presence of the collagen binding protein and the deficiency in 190 kDa protein results in a strong bound to collagen fibers and an accumulation of highly virulent bacteria to collagen-denuded vessels. Bacterial accumulation subsequently leads the activation of MMP-9 and inhibition of platelet aggregation in the damaged vessels, resulting in an acceleration of hemorrhage and hemorrhagic infarction (FIG. 16).

Among the patients infected with S. mutans, the rate of those who has been infected with strains expressing collagen binding protein is estimated to be 8 to 10% (Reference 16, 22). On the other hand, PA is normally expressed in most strains, and the strains as little as 4% do not express it (Reference 21). Accordingly, a S. mutans strain that expresses collagen binding protein and that is deficient in PA expression, i.e., a strain with an extremely high virulence is quite rare, and a limited number of strains become a potential risk factor of cerebral hemorrhage aggravation due to S. mutans bacteremia. Because the therapeutic approaches for cerebral hemorrhage are limited after its onset, prophylaxis is considered to be the most important approach (Reference 23). Accordingly, it is important to identify a patient who has been infected with a highly virulent S. mutans strain for the prevention of cerebral hemorrhage. In fact, the inventors has isolated CBP-expressing, highly virulent

TW295-type S. mutans from stroke patients with an extremely high frequency. Moreover, some of such strains also induced cerebral hemorrhage aggravation in a mouse model of hemorrhagic infarction, which indicates the relevance of a highly virulent S. mutans in the onset of hemorrhagic stroke.

From these results, it can be concluded that infection by a highly virulent, stroke-inducing S. mutans is a potential risk factor of stroke. Two important virulent factors of cerebral hemorrhage are the presence of collagen binding protein and the deficiency in PA expression, which are the common features shared by many of clinically isolated serotype k strains. Accordingly, the possession or deficiency of PA and/or CBP by a S. mutans strain can be an index for the determination of the risk at the hemorrhage in a carrier, which can be useful in prevention of cerebral hemorrhage.

### Detection Example 1

### Detection of Streptococcus Mutans Having a Cell Surface Layer Structure which may Becomes a Risk at Hemorrhage

### Materials and Methods

Tested Bacteria: Following Bacteria were Used in the Establishment of the Detection System.

| | |
|---|---|
| S. mutans | MT8148 strain (PA+/CBP−)/TW295 strain (PA−/CBP+) |
| S. sobrinus | B13 strain/6715 strain |
| S. sanguinis | ATCC10556 strain |
| S. oralis | ATCC10557 strain |
| S. gordonii | ATCC10558 strain |
| S. salivarius | HHT strain |

### Analysis 1. Method for Culturing S. Mutans (Mutans Streptococci)

(operation time: about 5 minutes, waiting time (such as during culturing of a bacterium):2 days)

Culturing of S. Mutans Employs Following Things:
spitz for collecting saliva (not particularly limited as long as it is sterilized and suitable for collecting and seeding)
a special dropper capable of collecting saliva of 10 μl
Special Medium A (agar medium) (24-well plate (it is not particularly limited as long as it is a plate of about 24-well, e.g., 24 well with Lid MICROPLATE (IWAKI)) coated with MSB agar medium e.g., Mitis-salivariusagar medium (Difco Laboratories) is supplemented with bacitracin (100 unit/ml; SIGMA-ALDRICH) and 15% sucrose (Wako Pure Chemical Industries, Ltd.). It is preferred to be provided with Anaero Pack®.)
a sterilized toothpick and the like for picking up bacterial colonies
Special Medium B (liquid medium) (sterilized Brain Heart Infusion (BHI) liquid medium (Difco Laboratories) contained in a disposal test tube)

Culturing of S. Mutans is Carried Out as Follows:

The saliva of the subject is collected in a small amount using the spitz for collecting saliva. 10 μl of the saliva is taken from the spitz using the special dropper, plated onto Special Medium A, then cultured at 37° C. for 48 hours, preferably in an anaerobic condition. After culturing, the presence of bacterial colonies is confirmed on gloss, colonies (rough colonies are desirable) are picked up and added into Special Medium B, cultured at 37° C. for 18 hours, and used in following Analyses 2, 3 and 4. Cultures of S. sobrinus, S. sanguinis, S. oralis, S. gordonii, and S. salivarius are used as controls, and in Analysis 1, it is confirmed that no bacterium other than S. mutans and S. sobrinus grows.

### Analysis 2. Method for Detecting S. Mutans (Mutans Streptococci)

(operation time: about 15 minutes, waiting time (such as during culturing of a bacterium):about 3 hours)

Although the method of culturing mutans streptococci of above Analysis 1 is provided with conditions in which the mutans streptococci group (S. mutans/S. sobrinus) can preferably grow, a bacterium having bacitracin-resistance other than mutans streptococci may grow. Therefore, confirmation is done in this step.

Detection Employs Following Things:
a special dropper capable of collecting bacterial solution of 10 μl
Special Medium C (96-well plate (e.g., MULTI WELL PLATE for ELISA (SUMIRON)) containing 100 μl of BHI solution containing 1% sucrose (Wako Pure Chemical Industries, Ltd.))
Wash Buffer A (PBS solution)
Buffer 1(a solution in which 0.5% crystal violet (Wako Pure Chemical Industries, Ltd.) is added to sterile distilled water)
Buffer 2 (7% acetate (Wako Pure Chemical Industries, Ltd.) solution)

Detection is Carried out as Follows:

10 μl of the bacterial solution cultured according to the method of Analysis 1 is added to Special Medium C, incubated at 37° C. for 3 hours. The Special Medium C is washed 3 times with Wash Buffer A, then left still for approximately 15 minutes after the last Wash Buffer A is added. Wash Buffer A is removed, and the Special Medium C is washed once again with the Wash Buffer A, then 100 μl Buffer 1 is added to the Special Medium C, left still for 1 minute. This is washed 3 times with Wash Buffer A, and 200 μl of Buffer 2 is added thereto.

It is determined to be S. mutans-positive if the color of the medium is changed, S. mutans-negative if the color of the medium is unchanged.

### Analysis 3. Method for Detecting PA-Deleted S. Mutans

(operation time: about 30 minutes, waiting time (such as during culturing of a bacterium): about 11 hours and 30 minutes)

Detection of PA-Deleted S. Mutans Employs Following Things:
Special Plate (96-well plate; MICROTEST U-Bottom (BECTON DICKINSON))
Wash Buffer B (a PBST solution in which 0.05% of Triton X-100 (Wako Pure Chemical Industries, Ltd.) is added to Wash Buffer A used in Analysis 2)
Buffer 3 (a mixture of Tris buffered saline, pH6.8, 100 mM dithiothreitol (Wako Pure Chemical Industries, Ltd.) and 20% glycerin (Wako Pure Chemical Industries, Ltd.))
Buffer 4 (a PBST solution supplemented with 5% skimmed milk (BECTON DICKINSON))
Buffer 5 (a PBST solution supplemented with 0.1% rabbit anti-PA antiserum (stored in our laboratory))
Buffer 6 (a PBST solution supplemented with 0.1% porcine anti-rabbit immunoglobulin antibody (Dakopatts))
Buffer 7 (a solution in which AP (100 mM 2-amino-2-hydroxymethyl-1,3-propanediol, 5 mM magnesium chloride, 100 mM sodium chloride) buffer is supplemented with NBT solution (Wako Pure Chemical Industries, Ltd.) at 0.6% final concentration and BCIP solution (Wako Pure Chemical Industries, Ltd.) at 0.33% final concentration.)

Detection of PA-Deleted S. Mutans is Carried Out as Follows:

### (1) Sample Preparation

To 100 μl of the bacterial solution cultured according to the method of Analysis 1 above, Buffer 3 is added, and immersed in boiling water for 10 minutes, and frozen if it is to be stored.

### (2) Detection of PA-Deleted S. Mutans

1) (1) 100 μl of the sample prepared as above is added to the Special Plate, left still overnight at 4° C.
2) The Special Plate was washed 3 times in Wash Buffer B, then 100 μl of Buffer 4 is added thereto, left still at room temperature for 1 hour.
3) The Special Plate was washed 3 times in Wash Buffer B, then 100 μl of Buffer 5 is added thereto, reacted at room temperature for 1 hour.
4) The Special Plate was washed 3 times in Wash Buffer B, then 100 μl of Buffer 6 is added thereto, reacted at room temperature for 1 hour.
5) The Special Plate was washed 3 times in Wash Buffer B, then 100 μl of Buffer 7 is added thereto, and after 15 minutes changes in the color of the solution are observed. It is determined to be PA-positive if the color of the solution is changed, PA-negative if the color of the solution is not changed. Cultures of S. sobrinus, S. sanguinis, S oralis, S gordonii, and S. salivarius are used as controls, and in Analysis 3, it is confirmed that no bacterium other than PA-carrying S. mutans shows a positive reaction.

### Analysis 4. Detection Method of CBP-Carrying S. Mutans

(operation time: about 30 minutes, waiting time (such as during culturing of a bacterium): about 3 hours and 30 minutes)

Detection of CBP-Carrying S. Mutans Employs the Followings:
Special Medium D (the Special Plate used in Analysis 3, to which a mixed solution of sterile distilled water supplemented with 0.6% acetate and Type I collagen (Sigma) in 9:1 ration was added.)
Wash Buffer A (the same buffer as that used in above Analysis 2 (detection method of S. mutans))
Buffer 8 (Wash Buffer A supplemented with 5% bovine albumin (Sigma))
Wash Buffer C (Wash Buffer A which is a PBST solution supplemented with 0.01% Tween 20 (Wako Pure Chemical Industries, Ltd.))
Buffer 9 (sterile distilled water supplemented with 25% formaldehyde (Wako Pure Chemical Industries, Ltd.))
Buffer 1 (the same buffer as that used in above Analysis 2)
Buffer 2 (the same buffer as that used in above Analysis 2)

Detection of CBP-Carrying S. Mutans is Carried Out as Follows:
(1) Special Medium D is washed three times with Wash Buffer A, then 200 μl of Buffer 8 is added thereto, and left still at 37° C. for 1 hour.
(2) Washed three times with Wash Buffer C, then 200 μl of the bacterial solution cultured according to the method of 1 described above is added thereto, and incubated at 37° C. for 2 hours.
(3) Washed three times with Wash Buffer A, then 200 μl of Buffer 9 is added thereto, and left still at room temperature for 30 minutes.
(4) Washed three times with Wash Buffer A, then 200 μl of Buffer 1 is added to the 96-well plate, and left still for 1 minute.
(5) Washed three times with Wash Buffer A, then 200 μl of Buffer 2 is added thereto.

It is determined to be CBP-positive if the color of the solution is changed, CBP-negative if the color of the solution is not changed. Cultures of S. sobrinus, S. sanguinis, S. oralis, S. gordonii, and S. salivarius are used as controls, and in Analysis 4, it is confirmed that no bacterium other than CBP-carrying S. mutans shows a positive reaction.

### Analysis Example 1

FIG. 11 is an example of the result of an analysis on whether the S. mutans in saliva samples (A, B and C) collected from 3 subjects are PA and/or CBP-carrying strains. As results of culturing saliva samples in Special Medium A (bacitracin-selection agar medium) in steps in Analysis 1, colony formation was confirmed in all of A, B and C. Formed colonies are picked up and cultured in Special Medium B at 37° C. for 18 hours. Moreover, cultures of S. sobrinus, S. sanguinis, S. oralis, S. gordonii, and S. salivarius were cultured similarly as controls, and in Analysis 1, it was confirmed that no bacterium other than S. mutans and S. sobrinus grew.

Subsequently, in steps in Analysis 2, the bacterial solution cultured in Analysis 1 was added to Special Medium C, incubated at 37° C. for 3 hours, washed with Wash Buffer A, then stained with Buffer 1 containing crystal violet. Since the buffer was changed to blue-violet in the medium in which samples A and B has been cultured, the presence of S. mutans was determined. As the buffer remained transparent in the medium in which sample C has been cultured, no presence of S. mutans was determined.

In steps in Analysis 3, Buffer 3 was added to each of the bacterial solutions of the samples A and B cultured in Analysis 1 and boiled for 10 minutes, and stored frozen. This was added to Special Plate (96-well plate MICROTEST U-Bottom(BECTON DICKINSON)), left still overnight at 4° C. After washing with Wash Buffer B, Buffer 4 was added and blocked at room temperature for 1 hour, then Buffer 5 containing rabbit anti-PA antiserum was added and reacted at room temperature for 1 hour. After washing with Wash Buffer B, Buffer 6 containing porcine anti-rabbit immunoglobulin antibody was added and reacted at room temperature for 1 hour. After washing with Wash Buffer B, Buffer 7 which contained an alkaline phosphatase reaction-detecting reagent was added, and after 15 minutes changes in the color of the solution were observed. Since the solution was changed to pink in the plate of the sample A, the presence of PA-carrying S. mutans was determined. As the color of the solution remained transparent for the sample B, no presence of PA-carrying S. mutans was determined. Similar analysis was performed using cultures of S. sobrinus, S. sanguinis, S. oralis, S. gordonii, and S. salivarius as controls, confirming that no bacterium other than the PA-carrying S. mutans showed a positive reaction.

In steps in Analysis 4, Buffer 8 containing 5% bovine albumin was added to the Special Medium D coated with Type I collagen (Sigma), and left still at 37° C. for 1 hour. After washing with Wash Buffer C, bacterial solution cultured in Analysis 1 was added and incubated at 37° C. for 2 hours. After washing with Wash Buffer A, Buffer 9 containing 25% formaldehyde was added, left still at room temperature for 30 minutes. After washing with Wash Buffer A, Buffer 1 was added and left still for 1 minute. After washing with Wash Buffer A, Buffer B was added and changes in the color of the solution were observed. Since the color of the solution remained transparent in the plate containing the sample A, no presence of CBP-carrying S. mutans was determined. As the color of the solution changed to blue-violet in the plate containing the sample B, the presence of CBP-carrying S. mutans was determined. Similar analysis was performed using cultures of S. sobrinus, S. sanguinis, S. rails, S. gordonii, and S. salivarius as controls, confirming that no bacterium other than the CBP-carrying S. mutans showed a positive reaction.

### Example 4

### Optimal Conditions for Culturing S. Mutans

In order to obtain a determination with higher accuracy in Analyses 2 to 4 above, it is considered to be important to culture S. mutans as many as possible in Analysis 1 and to ensure the contamination of bacteria other than S. mutans as little as possible. As conditions for culturing, (1) culturing in an aerobic condition/anaerobic condition, (2) antibiotics (bacitracin) concentration, and (3) nutrient (sucrose) concentration were investigated. FIG. 12 is a graph showing the percentage of S. mutans to total bacteria isolated when bacitracin was added to the MSB medium at (a) 1 eq. or (b) 5 eq. (assuming the amount of bacitracin in a conventional MSB medium is 1 eq.) and sucrose was added to the MSB medium at 1 to 4 eq.(assuming the amount of sucrose in a conventional MSB medium is 1 eq.). It was shown that S. mutans could be isolated at the highest concentration in an anaerobic condition, when 1 eq. of bacitracin and 1 eq. of sucrose were used. Accordingly, it was shown that in order to obtain a determination with higher accuracy, it is necessary to culture in a sealable container in an anaerobic condition (e.g., in a sealed pack to which Anaero Pack® is attached) in a medium supplemented with bacitracin and sucrose at the same concentration (approx. 100 unit/ml and 15%, respectively) contained in a conventional MSB medium.

### Example 5

### Stock Period of the Sample

We investigated the stock period of saliva usable for detection of a virulent S. mutans under the optimal conditions shown in Example 4 using saliva that has been kept for a certain time after being sampled.

FIG. 13 is a graph showing the separation rate of S. mutans when the saliva that had been kept for 0 to 6 months after being sampled was used to perform Analysis 1, assuming the separation rate of S. mutans that could be separated when a saliva serially diluted with a sterile physiological saline on the day of being collected was plated onto a MSB agar medium is 100%. Saliva that had been stored frozen at −20° C. after being sampled was used. Sample number: N=8, except the 1 to 2 months-aged sample (N=6). The result shows that it is desired to use the saliva as a sample preferably within 3 months, preferably within 2 months most preferably within 1 month.

The sequences of the protein, polypeptide and nucleic acid used herein are described in the attached sequence listings, as follows:

TABLE 6
Table of sequences

| SEQ ID No. | Species or strain | content of the sequence |
|---|---|---|
| 1 | S. mutans MT8148 | PA-amino acid |
| 2 | S. mutans MT8148 | PA-DNA |
| 3 | TW295 | CBP-amino acid |
| 4 | TW295 | CBP-DNA-ORF |
| 5 | S. mutans TW295 | CBD-amino acid |
| 6 | S. mutans TW295 | CBD-DNA |
| 7 | S. mutans TW871 | CBP-amino acid |
| 8 | S. mutans TW871 | CBP-DNA-ORF |
| 9 | S. mutans TW871 | CBD-amino acid |
| 10 | S. mutans TW871 | CBD-DNA |
| 11 | Artificial | S. mutans-primer F |
| 12 | Artificial | S. mutans-primer R |
| 13 | Artificial | S. mutans-CBD-primer F (cnm1F) |
| 14 | Artificial | S. mutans-CBP-primer R (cnm1R) |
| 15 | Artificial | S. mutans-PAC-primerF (pac-F) |
| 16 | Artificial | S. mutans-PAC-primer R (pac-R) |
| 17 | S. mutans LJ23 | PA-amino acid |
| 18 | S. mutans LJ23 | PA-DNA |
| 19 | S. mutans SA98 | PA-amino acid |
| 20 | S. mutans SA98 | PA-DNA |
| 21 | S. mutans | antigenI/II-amino acid |
| 22 | S. mutans | antigenI/II gene (spa)-DNA |
| 23 | Neisseria meningitidis | iron-binding protein-amino acid |
| 24 | Neisseria meningitidis | iron-binding protein gene (fbp) |
| | | DNA |
| 25 | S. mutans SA53 | CBP-amino acid |
| 26 | S. mutans SA53 | CBP-DNA-ORF |
| 27 | S. mutans SA53 | CBD-amino acid |
| 28 | S. mutans SA53 | CBD-DNA |
| 29 | S. mutans LJ32 | CBP-amino acid |
| 30 | S. mutans LJ32 | CBP-DNA-ORF |
| 31 | S. mutans LJ32 | CBD-amino acid |
| 32 | S. mutans LJ32 | CBD-DNA |

TABLE 7
References

| | |
|---|---|
| 1. | Murray. C. J. & Lopez A. D. Mortality by cause for eight regions of |
| | the world: Global Burden of Disease Study. Lancet 349, |
| | 1269-1276 (1997). |
| 2. | Donnan. G. A., Fisher. M., Macleod. M. & Davis. S. M. Stroke. |
| | Lancet 371, 1612-1623 (2008). |
| 3. | Broderick, J. et al. A guideline from the American Heart |
| | Association. American Stroke Association Stroke Council. |
| | High Blood Pressure Research Council, and the Quality of |
| | Care and Outcomes in Research Interdisciplinary Working |
| | Group. Stroke 38, 2001-2023 (2007). |
| 4. | Woo. D. et al. Genetic and environmental risk factors for |
| | intracerebral hemorrhage: preliminary results of a population- |
| | based study. Stroke 33, 1190-1195 (2002). |
| 5. | Emsley. H. C. & Tyrrell. P. J. Inflammation and infection in |
| | clinical stroke. J. Cereb. Blood Flow. Metab. 22, 1399-1419 |
| | (2002). |
| 6. | Moreillon, P. & Que, Y. A. Infective endocarditis. Lancet |
| | 363, 139-149 (2004). |
| 7. | Mylonakis. E. & Calderwood. S. B. Infective endocarditis |
| | in adults. N. Engl. J. Med. 345, 1318-1330 (2001). |
| 8. | Bayer. A. S. et al. Diagnosis and management of infective |
| | endocarditis and its complications. Circulation 98, |
| | 2936-2948 (1998). |
| 9. | Nakano. K.. Nomura. R.. Nakagawa. I.. Hamada, S. and |
| | Ooshima. T. Demonstration of Streptococcus mutans with a |
| | cell wall polysaccharide specific to a new serotype, k, in the |
| | human oral cavity. J. Clin. Microbiol. 42, 198-202 (2004). |
| 10. | Eishi, K. et al. Surgical management of infective |
| | endocarditis associated with cerebral complications. |
| | Multi-center retrospective study in Japan. J. Thorac. |
| | Cardiovasc. Surg. 110, 1745-1755 (1995). |
| 11. | Fujiwara. T. et al. Biochemical and genetic |
| | characterization of serologically untypable Streptococcus |
| | mutans strains isolated from patients with bacteremia. |
| | Eur. J. Oral Sci. 109, 330-334 (2001). |

TABLE 8

| | |
|---|---|
| 12. | Zhao. B. Q. et al. Essential role of endogenous tissue plaminogen |
| | activator through matrix metalloprotease 9 induction and expression |
| | on heparin-produced cerebral hemorrhage after cerebral |
| | ischemia in mice. Blood 103, 2610-2616 (2004). |
| 13. | Gursoy-Ozdemir. Y. et al. Cortical spreading depression |
| | activates and upregulates MMP-9. J. Clin. Invest. 113, |
| | 1447-1455 (2004). |
| 14. | Sato. Y. et al. Streptococcus mutans strains harboring |
| | collagen-binding adhesin. J. Dent. Res. 83, 534-539 (2004). |
| 15. | Nakano. K. et al. Detection of novel serotype k |
| | Streptococcus mutans in infective endocarditis patients. |
| | J. Med. Microbiol. 56, 1413-1415 (2007). |
| 16. | Nakano. K. et al. Streptococcus mutans clonal variation |
| | revealed by multilocus sequence typing. J. Clin. Microbiol. |
| | 45, 2616-2625 (2007). |
| 17. | Hampton. J. R. & Mitchell. J. R. A. Effect of aggregating |
| | agents on the electrophoretic mobility of human platelets. |
| | Br. Med. J. 1, 1074-1077 (1966). |
| 18. | Hampton. J. R. & Mitchell. J. R. A. Modification of the |
| | electrokinetic response of blood platelets to aggregating |
| | agents. Nature 210, 1000-1002 (1966). |
| 19. | Boisseau. M. R.. Lorient. M. F.. Born. G. V. R. & Michal. F. |
| | Change in electrophoretic mobility associated with the |
| | shape change of human blood platelets. Proc. R. Soc. Load. |
| | B Biol. Sci. 196, 471-474 (1977). |
| 20. | Okabashi. N.. Sasakawa. C.. Yoshikawa. M.. Hamada. |
| | S. & Koga. T. Cloning of a surface protein antigen gene |
| | from serotype c Streptococcus mutans. Mol. Microbiol. 3, |
| | 221-228 (1989). |
| 21. | Nakano. K.. Tsuji. M.. Nishimura. K.. Nomura. R. & |
| | Ooshima T. Contribution of cell surface protein antigen |
| | PAc of Streptococcus mutans to bacteremia. Microbes |
| | Infect. 8, 114-121 (2006). |
| 22. | Nomura. R. et al. Molecular and clinical analyses of |
| | the gene encoding the collagen-binding adhesin of |
| | Streptococcus mutans. J. Med. Microbiol. 58, 469-475 |
| | (2009). |

TABLE 9

| | |
|---|---|
| 23. | Ariesen. M. J.. Claus. S. P.. Rinkel. G. J. & Algra. A. Risk factors |
| | for intracerebral hemorrhage in the general population: a systematic |
| | review. Stroke 34, 2060-2065 (2003). |
| 24. | Ooshima. T.. Izumitani. A.. Sobue. S.. Okahashi. N. & Hamada. |
| | S. Non-cariogenicity of the disaccharide palatinose in experimental |
| | dental caries of rats. Infect. Immun. 39, 43-49 (1983). |
| 25. | Nakano. K. et al. Protein antigens in serotype k Strepococcus mutans |
| | clinical isolates. J. Dent. Res. 87, 964-968 (2008). |
| 26. | Nakano. K. et al. Molecular characterization of Streptococcus mutans |
| | strains containing the cnm gene encoding a collagen- |
| | binding adhesin. Arch. Oral Biol. (in press). |
| 27. | Waterhouse. J. C. & Russell R. R. B. Dispensable genes and |
| | foreign DNA in Streptococcus mutans. Microbiology 152, |
| | 1777-1788 (2006). |
| 28. | Suzuki. Y.. Nagai. N. & Collen. D. Comparative effects of |
| | microplasmin and tissue-type plasminogen activator (tPA) on |
| | cerebral hemorrhage in a middle cerebral artery occlusion model |
| | in mice. J. Thromb. Haemost. 2, 1617-1621 (2004). |
| 29. | Nakano K, Nomura R, Taniguchi N, Lapirattanakul J, Kojima A, |
| | Naka S, Senawongse P, Srisatjaluk R, Gr?nroos L, Alaluusua S, |
| | Matsumoto M, Ooshima T. Molecular characterization of |
| | Streptococcus mutans strains containing the cnm gene encoding |
| | a collagen-binding adhesin. Arch Oral Biol. 55(1): 34-9, 2010. |

1. A method of detecting a hemorrhage-aggravating oral bacterium, comprising a step of detecting Collagen Binding Protein (CBP) and/or cell surface charge of oral bacteria in a sample and determining the hemorrhage aggravating oral bacterium is present if CBP is detected and/or the cell surface charge is negative.

2. A method of screening a subject at a high risk of hemorrhage aggravation, comprising a step of determining the subject is at a high risk of hemorrhage aggravation if the hemorrhage aggravating oral bacterium is detected in a biological sample obtained from a subject by the method according to claim 1.

3. A method of judging the risk of hemorrhage aggravation in a subject, comprising a step of determining the subject is at a high risk of hemorrhage aggravation if the hemorrhage aggravating oral bacterium is detected in a biological sample obtained from a subject by the method according to claim 1.

4. The method according to claim 1, wherein the hemorrhage is hemorrhage by diabrosis.

5. The method according to claim 1, wherein the oral bacterium is Streptococcus mutans.

6. (canceled)

7. (canceled)

8. The method according to claim 1, wherein CBP is selected from the group consisting of:
(1) a polypeptide comprising an amino acid sequence according to SEQ ID NO. 5, 9, 27 or 31;
(2) a polypeptide comprising one or more mutations in the polypeptide of (1), but having an equal function to the polypeptide of (1);
(3) a polypeptide comprising an amino acid sequence encoded by a nucleic acid sequence that hybridizes with a nucleic acid sequence according to SEQ ID NOs. 6, 10, 28 or 32 or its complementary sequence or its fragment under stringent condition, and having an equal function as the polypeptide of (1); and
(4) a polypeptide comprising an amino acid sequence having 70% or more homology with an amino acid sequence according to SEQ ID NO. 5, 9, 27 or 31, and having an equal function to the polypeptide of (1).

9. The method according to claim 8, wherein CBP comprises a polypeptide consisting of an amino acid sequence according to SEQ ID NO. 5, 9, 27 or 31.

10. A reagent for the detection of a hemorrhage-aggravating oral bacterium, comprising an oral bacterial Collagen Binding Protein (CBP)-detecting agent.

11. (canceled)

12. A kit for the detection of a hemorrhage-aggravating oral bacterium in a subject, comprising at least:
a Collagen Binding Protein (CBP)-detecting agent.

13. (canceled)

14. (canceled)

15. An inhibitor of hemorrhage aggravation comprising a substance that binds to an oral bacterial Collagen Binding Protein (CBP) or to a nucleic acid encoding the CBP protein.

16. An agent for the detection of collagen-denuded site in tissue, comprising an oral bacterial Collagen Binding Protein (CBP).

17. A carrier for delivering a substance to the collagen-denuded site, comprising an oral bacterial Collagen Binding Protein (CBP).

18. A therapeutic agent for hemorrhage comprising an oral bacterial Collagen Binding Protein (CBP) and a hemostatic agent.

19. The therapeutic agent for hemorrhage according to claim 18, for a subject having low sensitivity of platelet to collagen.

20. A prophylactic agent for hemorrhage aggravation comprising an oral bacterium-removing agent.

21. A method according to claim 1, wherein a step further comprises detecting Protein Antigen (PA) of oral bacteria in a sample and determining as the hemorrhage aggravating oral bacterium is presented if PA is not detected.


SEQUENCE LISTING (32 sequences)

SEQ ID NO: 1 - protein, 1565 aa, Streptococcus mutans
MKVKKTYGFRKSKISKTLCGAVLGTVAAVSVAGQKVFADETTTTSDVDTKVVGTQTGNPATNLPEAQGSASKEAEQSQTKLERQMVHTIEVPKTDLDQAAKDAKSAGVNVVQDADVNKGTVKTPEEAVQKETEIKEDYTKQAEDIKKTTDQYKSDVAAHEAEVAKIKAKNQATKEQYEKDMAAHKAEVERINAANAASKTAYEAKLAQYQADLAAVQKTNAANQAAYQKALAAYQAELKRVQEANAAAKAAYDTAVAANNAKNTEIAAANEEIRKRNATAKAEYETKLAQYQAELKRVQEANAANEADYQAKLTAYQTELARVQKANADAKATYEAAVAANNAKNAALTAENTAIKQRNENAKATYEAALKQYEADLAAVKKANAANEADYQAKLTAYQTELARVQKANADAKAAYEAAVAANNAANAALTAENTAIKKRNADAKADYEAKLAKYQADLAKYQKDLADYPVKLKAYEDEQTSIKAALAELEKHKNEDGNLTEPSAQNLVYDLEPNANLSLTTDGKFLKASAVDDAFSKSTSKAKYDQKILQLDDLDITNLEQSNDVASSMELYGNFGDKAGWSTTVSNNSQVKWGSVLLERGQSATATYTNLQNSYYNGKKISKIVYKYTVDPKSKFQGQKVWLGIFTDPTLGVFASAYTGQVEKNTSIFIKNEFTFYHEDEKPINFDNALLSVTSLNREHNSIEMAKDYSGKFVKISGSSIGEKNGMIYATDTLNFKQGEGGSRWTMYKNSQAGSGWDSSDAPNSWYGAGAIKMSGPNNHVTVGATSATNVMPVSDMPVVPGKDNTDGKKPNIWYSLNGKIRAVNVPKVTKEKPTPPVKPTAPTKPTYETEKPLKPAPVAPNYEKEPTPPTRTPDQAEPNKPTPPTYETEKPLEPAPVEPSYEAEPTPPTRTPDQAEPNKPTPPTYETEKPLEPAPVEPSYEAEPTPPTPTPDQPEPNKPVEPTYEVIPTPPTDPVYQDLPTPPSDPTVHFHYFKLAVQPQVNKEIRNNNDINIDRTLVAKQSVVKFQLKTADLPAGRDETTSFVLVDPLPSGYQFNPEATKAASPGFDVTYDNATNTVTFKATAATLATFNADLTKSVATIYPTVVGQVLNDGATYKNNFTLTVNDAYGIKSNVVRVTTPGKPNDPDNPNNNYIKPTKVNKNENGVVIDGKTVLAGSTNYYELTWDLDQYKNDRSSADTIQKGFYYVDDYPEEALELRQDLVKITDANGNEVTGVSVDNYTNLEAAPQEIRDVLSKAGIRPKGAFQIFRADNPREFYDTYVKTGIDLKIVSPMVVKKQMGQTGGSYENQAYQIDFGNGYASNIVINNVPKINPKKDVTLTLDPADTNNVDGQTIPLNTVFNYRLIGGIIPANHSEELFEYNFYDDYDQTGDHYTGQYKVFAKVDITLKNGVIIKSGTELTQYTTAEVDTTKGAITIKFKEAFLRSVSIDSAFQAESYIQMKRIAVGTFENTYINTVNGVTYSSNTVKTTTPEDPADPTDPQDPSSPRTSTVIIYKPQSTAYQPSSVQETLPNTGVTNNAYMPLLGIIGLVTSFSLLGLKAKKD

SEQ ID NO: 2 - DNA, 4698 bp, Streptococcus mutans
atgaaagtcaaaaaaacttacggttttcgtaaaagtaaaattagtaaaacactgtgtggtgctgttctaggaacagtagcagcagtctctgtagcaggacaaaaggtttttgccgatgaaacgaccactactagtgatgtagatactaaagtagttggaacacaaactggaaatccagcgaccaatttgccagaggctcaagggagtgcgagtaaggaagctgaacaaagtcaaaccaagctggagagacaaatggttcataccattgaagtacctaaaactgatcttgatcaagcagcaaaagatgctaagtctgctggtgtcaatgttgtccaagatgccgatgttaataaaggaactgttaaaacacctgaagaagcagtccaaaaagaaactgaaattaaagaagattacacaaaacaagctgaggatattaagaagacaacagatcaatataaatcggatgtagctgctcatgaggcagaagttgctaaaatcaaagctaaaaatcaggcaactaaagaacagtatgaaaaagatatggcagctcataaagccgaggttgaacgcattaatgctgcaaatgctgccagtaaaacagcttatgaagctaaattggctcaatatcaagcagatttagcagccgttcaaaaaaccaatgctgccaatcaagcagcctatcaaaaagcccttgctgcttatcaggctgaactgaaacgtgttcaggaagctaatgcagccgccaaagccgcttatgatactgctgtagcagcaaataatgccaaaaatacagaaattgccgctgccaatgaagaaattagaaaacgcaatgcaacggccaaagctgaatatgagactaagttagctcaatatcaagctgaactaaagcgtgttcaggaagctaatgccgcaaacgaagcagactatcaagctaaattgaccgcctatcaaacagagcttgctcgtgttcaaaaagccaatgcggatgctaaagcgacctatgaagcagctgtagcagcaaataatgccaaaaatgcggcactcacagctgaaaatactgcaattaagcaacgcaatgagaatgctaaggcgacttatgaagctgcactcaagcaatatgaggccgatttggcagcggtgaaaaaagctaatgccgcaaacgaagcagactatcaagctaaattgaccgcctatcaaacagagctcgctcgcgttcaaaaagccaatgcggatgctaaagcggcctatgaagcagctgtagcagcaaataatgccgcaaatgcagcgctcacagctgaaaatactgcaattaagaagcgcaatgcggatgctaaagctgattacgaagcaaaacttgctaagtatcaagcagatcttgccaaatatcaaaaagatttagcagactatccagttaagttaaaggcatacgaagatgaacaaacttctattaaagctgcactggcagaacttgaaaaacataaaaatgaagacggaaacttaacagaaccatctgctcaaaatttggtctatgatcttgagccaaatgcgaacttatctttgacaacagatgggaagttccttaaggcttctgctgtggatgatgcttttagcaaaagcacttcaaaagcaaaatatgaccaaaaaattcttcaattagatgatctagatatcactaacttagaacaatctaatgatgttgcttcttctatggagctttatgggaattttggtgataaagctggctggtcaacgacagtaagcaataactcacaggttaaatggggatcggtacttttagagcgcggtcaaagcgcaacagctacatacactaacctgcagaattcttattacaatggtaaaaagatttctaaaattgtctacaagtatacagtggaccctaagtccaagtttcaaggtcaaaaggtttggttaggtatttttaccgatccaactttaggtgtttttgcttctgcttatacaggtcaagttgaaaaaaacacttctatttttattaaaaatgaattcactttctatcacgaagatgaaaaaccaattaattttgataatgcccttctctcagtgacttctcttaaccgtgaacataactctattgagatggctaaagattatagtggtaaatttgtcaaaatctctggttcatctattggtgaaaagaatggcatgatttatgctacagatactcttaactttaaacagggtgaaggtggctctcgctggactatgtataaaaatagtcaagctggttcaggatgggatagttcagatgcgccgaattcttggtatggagcaggggctattaaaatgtctggtccgaataaccatgttactgtaggagcaacttctgcaacaaatgtaatgccagtttctgacatgcctgttgttcctggtaaggacaatactgatggcaaaaaaccaaatatttggtattctttaaatggtaaaatccgtgcggttaatgttcctaaagttactaaggaaaaacccacacctccggttaaaccaacagctccaactaaaccaacttatgaaacagaaaagccattaaaaccggcaccagtagctccaaattatgaaaaggagccaacaccgccgacaaggacaccggatcaagcagagccaaacaaacccacaccgccgacctatgaaacagaaaagccgttggagccagcacctgttgagccaagctatgaagcagagccaacaccgccgacaaggacaccggatcaggcagagccaaataaacccacaccgccgacctatgaaacagaaaagccgttggagccagcacctgttgagccaagctatgaagcagagccaacgccaccgacaccaacaccagatcaaccagaaccaaacaaacctgttgagccaacttatgaggttattccaacaccgccgactgatcctgtttatcaagatcttccaacacctccatctgatccaactgttcatttccattactttaaactagctgttcagccgcaggttaacaaagaaattagaaacaataacgatattaatattgacagaactttggtggctaaacaatctgttgttaagttccagctgaagacagcagatctccctgctggacgtgatgaaaccacttcctttgtcttggtagatcccctgccatctggttatcaatttaatcctgaagctacaaaagctgcaagccctggctttgatgtcacttatgataatgcaactaatacagtcaccttcaaggcaactgcagcaactttggctacgtttaatgctgatttgactaagtcagtggcaacgatttatccaacagtggtcggacaagttcttaatgatggcgcaacttataagaataatttcacgctcacagtcaatgatgcttatggcattaaatccaatgttgttcgggtgacaactcctggtaaaccaaatgatccagataatccaaataataattatattaaaccaactaaggttaataaaaacgaaaatggcgttgttattgatggtaaaacagttcttgccggttcaacgaattattatgagctaacttgggatttggatcaatataaaaacgaccgctcttcagcagataccattcaaaaaggattttactatgtagatgattatccagaagaagcgcttgaattgcgtcaggatttagtgaagattacagatgctaatggtaatgaagttactggtgttagtgtggataattatactaatcttgaagcagcccctcaagaaattagagatgttctttctaaggcaggaattagacctaaaggtgctttccaaattttccgtgccgataatccaagagaattttatgatacttatgtcaaaactggaattgatttgaagattgtatcaccaatggttgttaaaaaacaaatgggacaaacaggcggcagttatgaaaatcaagcttaccaaattgactttggtaatggttatgcatcaaatatcgttatcaataatgttcctaagattaaccctaagaaagatgtgaccttaacacttgatccggctgatacaaataatgttgatggtcagactattccacttaatacagtctttaattaccgtttgattggtggcattatccctgcaaatcactcagaagaactctttgaatacaatttctatgatgattatgatcaaacaggagatcactatactggtcagtataaagtttttgccaaggttgatatcactcttaaaaacggtgttattatcaagtcaggtactgagttaactcagtatacgacagcggaagttgataccactaaaggtgctatcacaattaagttcaaggaagcctttctgcgttctgtttcaattgattcagccttccaagctgaaagttatatccaaatgaaacgtattgcggttggtacttttgaaaatacctatattaatactgtcaatggggtaacttacagttcaaatacagtgaaaacaactactcctgaggatcctgcagaccctactgatccgcaagatccatcatcaccgcggacttcaactgtaattatctacaaacctcaatcaactgcttatcagccaagctctgttcaagaaacattaccaaatacgggagtaacaaacaatgcttatatgcctttacttggtattattggcttagttactagttttagtttgcttggtttaaaggctaagaaagattga

SEQ ID NO: 3 - protein, 555 aa, Streptococcus mutans
MKRKGLRRLLKFFGTVAIILPMFFIALTKAQASDVSSNISSLTVSPTQINDGGKTTVRFEFDEHAQNIKAGDTITVNWQNSGTVRGTGYTKTIKLEVQGKYVGDLVVTQDKAVVTFNDSITGLQNITGWGEFEIEGRNFTDTTTGNTGSFQVTSGGKTAEVTVVKSASGTTGVFYYKTGDMQTDDTNHVRWFLNINNENAYVDSDIRIEDDIQSGQTLDIDSFDITVNGSESYHGQEGINQLAQRYGATISADPASGHISVYIPQGYASLNRFSIMYLTKVDNPDQKTFENNSKAWYKENGKDAVDGKEFNHSVANVNAAGGVDGRTTTTTEKPTTTTEAPTTTETPTTTEAPTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEVSSETTKAEETTTKVKEPEKTTTSVPAGTTSNKPNKPSGKQGAGTKGLPSTGEESGIVLSLLGLATVSVTGLVYRKYHS

SEQ ID NO: 4 - DNA, 1668 bp, Streptococcus mutans
atgaaaagaaaaggtttacgaagactattaaagttttttggaaccgttgccatcattttgccaatgtttttcatagctttaacgaaagctcaggcaagtgatgtcagcagtaacatttcatcgctgacggtatcaccgactcagattaatgatggcggtaagaccaccgttcgctttgagtttgatgagcatgctcaaaatattaaagcaggcgacaccattactgttaactggcagaattcaggaacagtcagaggaacaggttatacgaaaaccattaagctggaggttcagggcaagtatgttggtgatttggtagttacgcaagacaaagcagttgttactttcaatgacagtattactggcttgcagaatatcaccggctggggtgaatttgaaatcgaaggccggaattttactgacactactaccggaaatactggcagcttccaagttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcacggtcaagaaggtattaatcagcttgcccaaagatatggtgcaactatttcagctgatccggctagtggccatatcagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaatgttaatgccgccggcggtgtggacggaagaacaaccactactacagaaaagccaacaacgacgacagaggctccaacaacaacggaaactccaacgacaacagaggctccaacaacggaagctccaacgacaacagaggctccaacgacaacagaggctccaacaacaacggaagctccaacgacaacagaagctccaacaacaacggaagctccaacgacaacagaggctccaacgacaacagaggctccaacaacaacggaagctccaacgacaacagaggctccaacaacaacggaagctccaacgacaacagaagctccaacaacaacggaagctccaacgacaacagaggctccaacaacaacggaagctccaacgacaacagaggctccaacaacaacggaagctccaacgacaacagaggctccaacaacaacggaagctccaacaacaacggaagctccaacaacaacggaagtatcttcagaaacaactaaagctgaagaaacaactactaaagttaaggaaccagaaaaaacaacgacatcagttccagcaggtacaacttcaaacaaacctaataagccatcaggcaaacaaggtgctggtaccaagggacttccaagcacaggcgaagaaagcggtattgttttgtcacttctcggtcttgcaactgtctcagtgactggtctagtttaccgtaaatatcatagctga

SEQ ID NO: 5 - protein, 165 aa, Streptococcus mutans
VTSGGKTAEVTVVKSASGTTGVFYYKTGDMQTDDTNHVRWFLNINNENAYVDSDIRIEDDIQSGQTLDIDSFDITVNGSESYHGQEGINQLAQRYGATISADPASGHISVYIPQGYASLNRFSIMYLTKVDNPDQKTFENNSKAWYKENGKDAVDGKEFNHSVAN

SEQ ID NO: 6 - DNA, 495 bp, Streptococcus mutans
gttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcacggtcaagaaggtattaatcagcttgcccaaagatatggtgcaactatttcagctgatccggctagtggccatatcagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaat

SEQ ID NO: 7 - protein, 549 aa, Streptococcus mutans
MKRKGLRRLLKFFGTVAIILPMFFIALTKAQASDVSSNISSLTVSPTQINDGGKTTVRFEFDEHAQNIKAGDTITVNWQNSGTVRGTGYTKTIKLEVQGKYVGDLVVTQDKAVVTFNDSITGLQNITGWGEFEIEGRNFTDTTTGSTGSFQVTSGGKTAEVTVVKSASGTTGVFYYKTGDMQTDDTNHVRWFLNINNENAYVDSDIRIEDDIQSGQTLDIDSFDITVNGSESYHGQEGINQLAQRYGATISADPASGHNSVYIPQGYASLNRFSIMYLTKVDNPDQKTFENNSKAWYKENGKDAVDGKEFNHSVANVNAAGGVDGRTTTTTEKPTTTTEAPTTTETPTTTEAPTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTEVSSETTKAEETTTKVKEPEKTTTSVPAGTTSNKPNKPSGKQGAGTKGLPSTGEESGIVLSLLGLATVSVTGLVYRKYHS

SEQ ID NO: 8 - DNA, 1650 bp, Streptococcus mutans
atgaaaagaaaaggtttacgaagactattaaagttttttggaaccgttgccatcattttgccaatgtttttcatagctttaacgaaagctcaggcaagtgatgtcagcagtaacatttcatcgctgacggtatcaccgactcagattaatgatggcggtaagaccaccgttcgctttgagtttgatgagcatgctcaaaatattaaagcaggcgacaccattactgttaactggcagaattcaggaacagtcagaggaacaggttatacgaaaaccattaagctggaggttcagggcaagtatgttggtgatttggtagttacgcaagacaaagcagttgttactttcaatgacagtattactggcttgcagaatatcaccggctggggtgaatttgaaatcgaaggccggaattttactgacactactaccggaagtactggcagcttccaagttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcacggtcaagaaggtattaatcagcttgcccaaagatatggtgcaactatttcagctgatccggctagtggccataacagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaatgttaatgccgccggcggtgtggacggaagaacaaccactactacagaaaagccaacaacgacgacagaggctccaacaacaacggaaactccaacgacaacagaggctccaacaacggaagctccaacgacaacagaggctccaacaacaacggaagctccaacgacaacagaagctccaacaacaacggaagctccaacgacaacagaggctccaacaacaacggaagctccaacgacaacagaagctccaacaacaacggaagctccaacgacaacagaggctccaacaacaacggaagctccaacgacaacagaagctccaacgacaacagaggctccaacgacaacagaagctccaacaacaacggaagctccaacgacaacagaggctccaacaacaacggaagctccaacgacaacagaggctccaacaacaacggaagctccaacaacaacggaagtatcttcagaaacaactaaagctgaagaaacaactactaaagttaaggaaccagaaaaaacaacgacatcagttccagcaggtacaacttcaaacaaacctaataagccatcaggcaaacaaggtgctggtaccaagggacttccaagcacaggcgaagaaagcggtattgttttgtcacttctcggtcttgcaactgtctcagtgactggtctagtttaccgtaaatatcatagctga

SEQ ID NO: 9 - protein, 165 aa, Streptococcus mutans
VTSGGKTAEVTVVKSASGTTGVFYYKTGDMQTDDTNHVRWFLNINNENAYVDSDIRIEDDIQSGQTLDIDSFDITVNGSESYHGQEGINQLAQRYGATISADPASGHNSVYIPQGYASLNRFSIMYLTKVDNPDQKTFENNSKAWYKENGKDAVDGKEFNHSVAN

SEQ ID NO: 10 - DNA, 495 bp, Streptococcus mutans
gttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcacggtcaagaaggtattaatcagcttgcccaaagatatggtgcaactatttcagctgatccggctagtggccataacagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaat

SEQ ID NO: 11 - DNA, 27 bp, Artificial
  S.mutans specific primer F
ggcaccacaacattgggaagctcagtt

SEQ ID NO: 12 - DNA, 25 bp, Artificial
  S.mutans specific primer R
ggaatggccgctaagtcaacaggat

SEQ ID NO: 13 - DNA, 20 bp, Artificial
  S.mutans CBP primer (cnm1F)
gacaaagaaatgaaagatgt

SEQ ID NO: 14 - DNA, 20 bp, Artificial
  S.mutans CBP primer (cnm1R)
gcaaagactcttgtccctgc

SEQ ID NO: 15 - DNA, 30 bp, Artificial
  S.mutans PAC primer (pac-F)
gcgcgcatgctttattcagatttggaggat

SEQ ID NO: 16 - DNA, 30 bp, Artificial
  S.mutans PAC primer (pac-R)
gcgaaagcgcatgctgtgatttatcgcttc

SEQ ID NO: 17 - protein, 1566 aa, Streptococcus mutans
MKVKKTYGFRKSKISKTLCGAVLGTVAAVSVAGQKVFADETTTTSDVDTKVVGTQTGNPATNLPEAQGSASKEAEQSQNQAGETNGSIPVEVPKTDLDQAAKDAKSAGVNVVQDADVNKGTVKTAEEAVQKETEIKEDYTKQAEDIKKTTDQYKSDVAAHEAEVAKIKAKNQATKEQYEKDMAAHKAEVERINAANAASKTAYEAKLAQYQADLAAVQKTNAANQAAYQKALAAYQAELKRVQEANAAAKAAYDTAVAANNAKNTEIAAANEEIRKRNATAKAEYETKLAQYQAELKRVQEANAANEADYQAKLTAYQTELARVQKANADAKAAYEAAVAANNAKNAALTAENTAIKQRNENAKATYEAALKQYEADLAAVKKANAANEADYQAKLTAYQTELARVQKANADAKAAYEAAVAANNAANAALTAENTAIKKRNADAKADYEAKLAKYQADLAKYQKDLADYPVKLKAYEDEQASIKAALAELEKHKNEDGNLTEPSAQNLVYDLEPNANLSLTTDGKFLKASAVDDAFSKSTSKAKYDQKILQLDDLDITNLEQSNDVASSMELYGNFGDKAGWSTTVSNNSQVKWGSVLLERGQSATATYTNLQNSYYNGKKISKIVYKYTVDPKSKFQGQKVWLGIFTDPTLGVFASAYTGQVEKNTSIFIKNEFTFYDEDGKPIDFDNALLSVASLNREHNSIEMAKDYSGKFVKISGSSIGEKNGMIYATDTLNFKQGEGGSRWTMYKNSQAGSGWDSSDAPNSWYGAGAIKMSGPNNHVTVGATSATNVMPVSDMPVVPGKDNTDGKKPNIWYSLNGKIRAVNVPKVTKEKPTPPVKPTAPTKPTYETEKPLKPAPVAPNYEKEPTPPTRTPDQAEPNKPTPPTYETEKPLEPAPVEPSYEAEPTPPTRTPDQAEPNKPTPPTYETEKPLEPAPVEPSYEAEPTPPTPTPDQPEPNKPVEPTYEVIPTPPTDPVYQDLPTPPSVPTVHFHYFKLAVQPQVNKEIRNNNDVNIDRTLVAKQSVVKFQLKTADLPAGRDETTSFVLVDPLPSGYQFNPEATKAASPGFDVAYDNATNTVTFKATAATLATFNADLTKSVATIYPTVVGQVLNDGATYKNNFTLTVNDAYGIKSNVVRVTTPGKPNDPDNPNNNYIKPTKVNKNENGVVIDGKTVLAGSTNYYELTWDLDQYKNDRSSADTIQQGFYYVDDYPEEALELRQDLVKITDANGNEVTGVSVDNYTSLEAAPQEIRDVLSKAGIRPKGAFQIFRANNPREFYDTYVKTGIDLKIVSPMVVKKQMGQTGGSYENQAYQIDFGNGYASNIVINNVPKINPKKDVTLTLDPADTNNVDGQTIPLNTVFNYRLIGGIIPANHSEELFEYNFYDDYDQTGDHYTGQYKVFAKVDITLKNGVIIKSGTELTQHTTAEVDTTKGAITIKFKEAFLRSVSIDSAFQAESYIQMKRIAVGTFENTYINTVNGVTYSSNTVKTTTPEDPTDPTDPQDPSSPRTSTVINYKPQSTAYQPSSVQKTLPNTGVTNNAYMPLLGIIGLVTSFSLLGLKAKKD

SEQ ID NO: 18 - DNA, 4701 bp, Streptococcus mutans
atgaaagtcaaaaaaacttacggttttcgtaaaagtaaaattagtaaaacactgtgtggtgctgttctaggaacagtagcagcagtctctgtagcgggacaaaaggtttttgccgatgaaacgaccactactagtgatgtagatactaaagtagttggaacacaaactggaaatccagcgaccaatttgccagaggctcaagggagtgcgagtaaggaagctgaacaaagtcaaaaccaagctggagagacaaatggttcaataccagttgaagtacctaaaactgatcttgatcaagcagcaaaagatgctaagtctgctggtgtcaatgttgtccaagatgccgatgttaataaaggaactgttaaaacagctgaagaagcagtccaaaaagaaactgaaattaaagaagattacacaaaacaagctgaggatattaagaagacaacagatcaatataaatcggatgtagctgctcatgaggcagaagttgctaaaatcaaagctaaaaatcaggcgactaaagaacagtatgaaaaagatatggcagctcataaagccgaggttgaacgcattaatgctgcaaatgctgccagtaaaacagcttatgaagctaaattggctcaatatcaagcagatttagcagccgttcaaaaaaccaatgctgccaatcaagcagcctatcaaaaagcccttgctgcctatcaggctgaactgaaacgtgttcaggaagctaatgcagccgccaaagctgcttatgatactgctgtagcagcaaataatgccaaaaatacagaaattgccgctgccaatgaagaaattagaaaacgcaatgcaacggccaaagctgaatatgagactaagttagctcaatatcaagctgaactaaagcgtgttcaggaagctaatgccgcaaacgaagcagactatcaagctaaattgaccgcctatcaaacagagctcgctcgcgttcaaaaggctaatgcggatgctaaagcggcctatgaagcagctgtagcagcaaataatgccaaaaatgcggcactcacagctgaaaatactgcaattaagcaacgcaatgagaatgctaaggcgacttatgaagctgcactcaagcaatatgaggccgatttggcagcggtgaaaaaagctaatgccgcaaacgaagcagactatcaagctaaattgaccgcctatcaaacagagctcgctcgcgttcaaaaagccaatgcggatgctaaagcggcctatgaagcagctgtagcagcaaataatgccgcaaatgcagcgctcacagctgaaaatactgcaattaagaagcgcaatgcggatgctaaagctgattacgaagcaaaacttgctaagtatcaagcagatcttgccaaatatcagaaagatttagcagactatccagttaagttaaaggcatacgaagatgaacaagcttctattaaagctgcactggcagaacttgaaaaacataaaaatgaagacggaaacttaacagaaccatctgctcaaaatttggtctatgatcttgagccaaatgcgaacttatctttgacaacagatgggaagttccttaaggcttctgctgtggatgatgcttttagcaaaagcacttcaaaagcaaaatatgaccaaaaaattcttcaattagatgacctagatataactaacttagaacaatctaatgatgttgcttcttctatggagctttatgggaattttggtgataaagctggctggtcaacaacagtaagcaataactcacaggttaaatggggatcggtacttttagagcgcggtcaaagcgcaacagctacatacactaacctgcagaattcttattacaatggtaaaaagatttctaaaattgtctacaagtatacagtggaccctaagtccaagtttcaaggtcaaaaggtttggttaggtatttttaccgatccaactttaggtgtttttgcttccgcttatacaggtcaagttgaaaaaaacacttctatttttattaaaaatgaattcactttctatgacgaagatggaaaaccaattgattttgataatgcccttctctcagtagcttctcttaaccgtgaacataactctattgagatggctaaagattatagtggtaaatttgtcaaaatctctggttcatctattggtgaaaagaatggcatgatttatgctacagatactcttaactttaaacagggtgaaggcggctctcgctggactatgtataaaaatagtcaagctggttcaggatgggatagttcagatgcgccgaattcttggtatggagcaggggctattaaaatgtctggtccgaataaccatgttactgtaggagcaacttctgcaacaaatgtgatgccagtttctgacatgcctgttgttcctggtaaggacaatactgatggcaaaaaaccaaatatttggtattctttaaatggtaaaatccgtgcggttaatgttcctaaagttactaaggaaaaacccacacctccggttaaaccaacagctccaactaaaccaacttatgaaacagaaaagccattaaaaccggcaccagtagctccaaattatgaaaaggagccaacaccgccgacaaggacaccggatcaagcagagccaaataaacccacaccgccgacctatgaaacagaaaagccgttggagccagcacctgttgagccaagctatgaagcagagccaacaccgccgacaaggacaccggatcaggcagagccaaataaacccacaccgccgacctatgaaacagaaaagccgttggagccagcacctgttgagccaagctatgaagcagagccaacgccaccgacaccaacaccagatcaaccagaaccaaacaaacctgttgagccaacttatgaggttattccaacaccgccgactgatcctgtttatcaagatcttccaacacctccatctgtaccaactgttcatttccattactttaaactagctgttcagccgcaggttaacaaagaaattagaaacaataacgatgttaatattgacagaactttggtggctaaacaatctgttgttaagttccagctgaagacagcagatctccctgctggacgtgatgaaacaacttcctttgtcttggtagatcccctgccatctggttatcaatttaatcctgaagctacaaaagctgccagccctggctttgatgtcgcttatgataatgcaactaatacagtcaccttcaaggcaactgcagcaactttggctacgtttaatgctgatttgactaagtcagtggcaacgatttatccaacagtggtcggacaagttcttaacgatggcgcaacttataagaataatttcacactcacagtcaatgatgcttatggcattaaatccaatgttgttcgggtgacaactcctggtaaaccaaatgatccagataacccaaataataattatattaaaccaactaaggttaataaaaacgaaaatggcgttgttattgatggtaaaacagttcttgccggttcaacgaattattatgagctaacttgggatttggatcaatataagaacgaccgctcttcagcagataccattcaacaaggattttactatgtagatgattatccagaagaagcgcttgaattgcgtcaggatttagtgaagattacagatgctaatggtaatgaagttactggtgttagtgtggataattatactagtcttgaagcagcccctcaagaaattagagatgttctttctaaggcaggaattagacctaaaggtgctttccaaattttccgtgccaataatccaagagaattttatgatacttatgtcaaaactggaattgatttgaagattgtatcaccaatggttgttaaaaaacaaatgggacaaacaggtggcagttatgaaaatcaagcttaccaaattgactttggtaatggttatgcatcaaatatcgttatcaataatgttcctaagattaaccctaagaaagatgtgaccttaacacttgatccggctgatacaaataatgttgatggtcagactattccacttaatacagtctttaattaccgtttgattggtggcattatccctgcaaatcactcagaagaactctttgaatacaatttctatgatgattatgatcaaacaggagatcactatactggtcagtataaagtttttgccaaggttgatatcactcttaaaaacggtgttattattaagtcaggtactgaattgactcagcatacgacagcggaagttgataccactaaaggtgctatcacaattaagttcaaggaagcctttctgcgttctgtttcaattgattcagccttccaagctgaaagttatatccaaatgaaacgtattgcggttggtacttttgaaaatacttatattaatactgtcaatggggtaacttacagttcaaatacagtgaaaacaactactcctgaggatcctacagaccctactgatccgcaagatccatcatcaccgcggacttcaactgtaattaactacaaacctcaatcaactgcttatcaaccaagctctgtccaaaaaacgttaccaaatacgggagtaacaaacaatgcttatatgcctttacttggtattattggcttagttactagttttagtttgcttggcttaaaggctaagaaagattga

SEQ ID NO: 19 - protein, 1564 aa, Streptococcus mutans
MKVKKTYGFRKSKISKTLCGAVLGTVAAVSVAGQKVFADETTTTSDVDTKVVGTQTGNPATNLPEAQGSASKEAEQSQNQAGETNGSIPIEVPKTDLDQTAKDAKSAGVNVVQDADVNKGTVKTAEAAVQKETEIKEDYTKQAEDIKKTTDQYKSDVAAHEAEVAKIKAKNQATKEQYEKDMAAHKAEVERINAANAASKTAYEAKLAQYQADLAAVQKTNAANQAAYQKALAAYQAELKRVQEANAAAKAAYDTAVAANNAKNTEITAANEEIRKRNATAKAEYETKLAQYQAELKRVQEANAANEADYQAKLTAYQTELARVQKANADAKAAYEAAVAANNAKNAALTAENTAIKQRNENAKATYEAALKQYEADLAAAKKANAANEADYQAKLTAYQTELARVQKTNADAKAAYEAAVAANNAANAALTAENTAIKKRNADAKADYEAKLAKYQADLAKYQKDLADYPVKLKAYEDEQASIKAALAELEKHKNEDGNLTEPSAQNLVYDLEPNANLSLTTDGKFLKASAVDNAFKQDTNQYSKKNLQLDNLNVKYLENAGATASSMELYGNIGDKSSWTTNVGNKTEVKWGSVLLERGQSATATYTNLQNSYYNGKKISKIVYKYTVDPKSKFQGQKVWLGIFTDPTLGVFASAYTGQVEKNTSIFIKNEFTFYDEDGKPIDFDNALLSVASLNREHNSIEMAKDYSGKFVKISGSSIGEKNGMIYATDTLNFKQGEGGSRWTMYKNSQAGSGWDSSDAPNSWYGAGAIKMSGPNNHVTVGATSATNVMPVSDMPVVPGKDNTDGKKPNIWYSLNGKIRAVNVPKVTKEKPTPPVKPTAPTKPTYETEKPLKPAPVAPNYEKEPTPPTRTPDQAEPNKPTPPTYETEKPLEPAPVEPSYEAEPTPPTRTPDQAEPNKPTPPTYETEKPLEPAPVEPSYEAEPTPPTPTPDQPEPNKPVEPTYEVIPTPPTDPVYQDLPTPPSVPTVHFHYFKLAVQPQVNKEIRNNNDVNIDRTLVAKQSVVKFQLKTADLPAGRDETTSFVLVDPLPSGYQFNPEATKAASPGFDVTYDNATNTVTFKATAATLATFNADLTKSVATIYPTVVGQVLNDGATYKNNFTLTVNDAYGIKSNVVRVTTPGKPNDPDNPNNNYIKPTKVNKNENGVVIDGKTVLAGSTNYYELTWDLDQYKNDRSSADTIQKGFYYVDDYPEEALELRQDLVKITDANGNEVTGVSVDNYTSLEAAPQEIRDVLSKAGIRPKGAFQIFRADNPREFYDTYVKTGIDLKIVSPMVVKKQMGQTGGSYENQAYQIDFGNGYASNIVINNVPKINPKKDVTLTLDPADTNNVDGQTIPLNTVFNYRLIGGIIPANHSEELFEYNFYDDYDQTGDHYTGQYKVFAKVDITFKDGSIIKSGAELTQYTTAEVDTTKGAITIKFKEAFLRSVSIDSVFQAESYIQMKRIAVGTFENTYINTVNGVTYSSNTVKTTTPEDPTDPTDPQDPASPRTSTVINYKPQSTAYQPSSVQKTLPNTGVTNNAYMPLLGIIGLVTSFSLLGLKAKKD

SEQ ID NO: 20 - DNA, 4695 bp, Streptococcus mutans
atgaaagtcaaaaaaacttacggttttcgtaaaagtaaaattagtaaaacactgtgtggtgctgttctaggaacagtagcagcagtctctgtagcaggacaaaaggtttttgccgatgaaacgaccactactagtgatgtagatactaaagtagttggaacacaaactggaaatccagcgaccaatttgccagaggctcaagggagtgcgagtaaggaagctgaacaaagtcaaaaccaagctggagagacaaatggttcaataccaattgaagtacctaaaactgatcttgatcaaacagcaaaagatgctaagtctgctggtgtcaatgttgtccaagatgccgatgttaataaaggaactgttaaaacagctgaagcagcagtccaaaaagaaactgaaattaaagaagattacacaaaacaagctgaggatattaagaagacaacagatcaatataaatcggatgtagctgctcatgaggcagaagttgctaaaatcaaagctaaaaatcaggcaactaaagaacagtatgaaaaagatatggcagctcataaagccgaggttgaacgcattaatgctgcaaatgctgccagtaaaacagcttatgaagctaaattggctcaatatcaagcagatttagcagccgttcaaaaaaccaatgctgccaatcaagcagcctatcaaaaagcccttgctgcttatcaggctgaactgaagcgtgttcaggaagctaatgcagccgccaaagccgcttatgatactgctgtagcagcaaataatgccaaaaatacagaaattaccgctgccaatgaagaaattagaaaacgcaatgcaacggccaaagctgaatatgagactaagttagctcaatatcaagctgaactaaagcgtgttcaggaagctaatgcagcaaacgaagcagactatcaagctaaattgactgcttatcaaacagagctcgctcgcgttcaaaaggccaatgcggatgctaaagcggcctatgaagcagctgtagcagcaaataatgccaaaaatgcggcactcacagctgaaaatactgcaattaagcaacgcaatgagaatgctaaggcgacttatgaagctgcactcaagcaatatgaggccgatttggcagcagcgaaaaaagctaatgcagcaaacgaagcagactatcaagctaaattgaccgcttatcaaacagagctcgctcgcgttcaaaagaccaatgcggatgctaaagcggcctatgaagcagctgtagcagcaaataatgccgcaaatgcagcgctcacagctgaaaatactgcaattaagaagcgcaatgcggatgctaaagctgattacgaagcaaaacttgctaagtatcaagcagatcttgccaaatatcaaaaagatttagcagactatccagttaagttaaaggcatacgaagatgaacaagcttctattaaagctgcactggcagaacttgaaaaacataaaaatgaagacggaaacttaacagaaccatctgctcaaaatttggtctatgatcttgagccaaatgcgaacttatctttgacaacagatgggaagttccttaaggcttctgctgtggataacgcatttaagcaagatacaaatcaatatagtaaaaagaaccttcaattagataaccttaatgttaaatatctagaaaacgcaggagccactgcctcatctatggaattatacggaaatataggtgataaatcgagttggacaacaaatgtaggcaacaaaacagaagttaaatggggatcggtacttttagagcgcggtcaaagcgcaacagctacatacactaacctgcagaattcttattacaatggtaaaaagatttctaaaattgtctacaagtatacagtggaccctaagtccaagtttcaaggtcaaaaggtttggttaggtatttttaccgatccaactttaggtgtttttgcttccgcttatacaggtcaagttgaaaaaaacacttctatttttattaaaaatgaattcactttctatgacgaagatggaaaaccaattgattttgataatgcccttctctcagtagcttctcttaaccgtgaacataactctattgagatggctaaagattatagtggtaaatttgtcaaaatctctggttcatctattggtgaaaagaatggcatgatttatgctacagatactcttaactttaaacagggtgaaggcggctctcgctggactatgtataaaaatagtcaagctggttcaggatgggatagttcagatgcgccgaattcttggtatggagcaggggctattaaaatgtctggtccgaataaccatgttactgtaggagcaacttctgcaacaaatgtgatgccagtttctgacatgcctgttgttcctggtaaggacaatactgatggcaaaaaaccaaatatttggtattctttaaatggtaaaatccgtgcggttaatgttcctaaagttactaaggaaaaacccacacctccggttaaaccaacagctccaactaaaccaacttatgaaacagaaaagccattaaaaccggcaccagtagctccaaattatgaaaaggagccaacaccgccgacaaggacaccggatcaagcagagccaaataaacccacaccgccgacctatgaaacagaaaagccgttggagccagcacctgttgagccaagctatgaagcagagccaacaccgccgacaaggacaccggatcaggcagagccaaataaacccacaccgccgacctatgaaacagaaaagccgttggagccagcacctgttgagccaagctatgaagcagagccaacgccaccgacaccaacaccagatcaaccagaaccaaacaaacctgttgagccaacttatgaggttattccaacaccgccgactgatcctgtttatcaagatcttccaacacctccatctgtaccaactgttcatttccattactttaaactagctgttcagccgcaggttaacaaagaaattagaaacaataacgatgttaatattgacagaactttggtggctaaacaatctgttgttaagttccagctgaagacagcagatctccctgctggacgtgatgaaacaacttcctttgtcttggtagatcccctgccatctggttatcaatttaatcctgaagctacaaaagctgcaagccctggctttgatgtcacttatgataatgcaactaatacagtcaccttcaaggcaactgcagcaactttggctacgtttaatgctgatttgactaagtcagtggcaacgatttatccaacagtggtcggacaagttcttaatgatggcgcaacttataagaataatttcacgctcacagtcaatgatgcttatggcattaaatccaatgttgttcgggtgacaactcctggtaaaccaaatgatccagataatccaaataataattatattaaaccaactaaggttaataaaaacgaaaatggcgttgttattgatggtaaaacagttcttgccggttcaacgaattattatgagctaacttgggatttggatcaatataaaaacgaccgctcttcagcagataccattcaaaaaggattttactatgtagatgattatccagaagaagcgcttgaattgcgtcaggatttagtgaagattacagatgctaatggtaatgaagttactggtgttagtgtggataattatactagtcttgaagcagcccctcaagaaattagagatgttctttctaaggcaggaattagacctaaaggtgctttccaaattttccgtgccgataatccaagagaattttatgatacttatgtcaaaactggaattgatttgaagattgtatcaccaatggttgttaaaaaacaaatgggacaaacaggcggcagttatgaaaatcaagcttaccaaattgactttggtaatggttatgcatcaaatatcgttatcaataatgttcctaagattaaccctaagaaagatgtgaccttaacacttgatccggctgatacaaataatgttgatggtcagactattccacttaatacagtctttaattaccgtttgattggtggcattatccctgcaaatcactcagaagaactctttgaatacaatttctatgatgattatgatcaaacaggagatcactatactggtcagtataaagtttttgccaaggttgatatcacttttaaagacggttctattatcaagtcaggtgctgagttaactcagtatacgacagcggaagttgataccactaaaggtgctatcacaattaagttcaaggaagcctttctgcgttctgtttcaattgattcagtcttccaagctgaaagttatatccaaatgaaacgtattgcggttggtacttttgaaaatacttatattaatactgtcaatggggtaacttacagttcaaatacagtgaaaacaactactcctgaggatcctacagaccctactgatccgcaagatccagcatcaccgcggacttcaactgtaattaactacaaacctcaatcaactgcttatcaaccaagctctgtccaaaaaacgttaccaaatacgggagtaacaaacaatgcttatatgcctttacttggtattattggcttagttactagttttagtttgcttggcttaaaggctaagaaagattga

SEQ ID NO: 21 - protein, 1561 aa, Streptococcus mutans
MKVKKTYGFRKSKISKTLCGAVLGTVAAVSVAGQKVFADETTTTSDVDTKVVGTQTGNPATNLPEAQGSASKQAEQSQTKLERQMVHTIEVPKTDLDQAAKDAKSAGVNVVQDADVNKGTVKTAEEAVQKETEIKEDYTKQAEDIKKTTDQYKSDVAAHEAEVAKIKAKNQATKEQYGKDMVAHKAEVERINAANAASKTAYEAKLAQYQADLAAVQKTNAANQASYQKALAAYQAELKRVQEANAAAKAAYDTAVAANNAKNTEIAAANEEIRKRNATAKAEYETKLAQYQAELKRVQEANAANEADYQAKLTAYQTELARVQKANADAKAAYEAAVAANNAKNAALTAENTAIKQRNENAKATYEAALKQYEADLAAVKKANAANEADYQAKLTAYQTELARVQKANADAKAAYEAAVAANNAANAALTAENTAIKKRNADAKADYEAKLAKYQADLAKYQKDLADYPVKLKAYEDEQASIKAALAELEKHKNEDGNLTEPSAQNLVYDLEPNANLSLTTDGKFLKASAVDDAFSKSTSKAKYDQKILQLDDLDITNLEQSNDVASSMELYGNFGDKAGWSTTVSNNSQVKWGSVLLERGQSATATYTNLQNSYYNGKKISKIVYKYTVDPKSKFQGQKVWLGIFTDPTLGVFASAYTGQVEKNTSIFIKNEFTFYDEDGKPINFDNALLSVASLNRENNSIEMAKDYTGKFVKISGSSIGEKNGMIYATDTLNFRQGQGGARWTMYTRASEPGSGWDSSDAPNSWYGAGAIRMSGPNNSVTLGAISSTLVVPADPTMAIETGKKPNIWYSLNGKIRAVNLPKVTKEKPTPPVKPTAPTKPTYETEKPLKPAPVAPNYEKEPTPPTRTPDQAEPKKPTPPTYETEKPLEPAPVEPSYEAEPTPPTRTPDQAEPNKPTPPTYETEKPLEPAPVEPSYEAEPTPPTPTPDQPEPNKPVEPTYEVIPTPPTDPVYQDLPTPPSIPTVHFHYFKLAVQPQVNKEIRNNNDVNIDRTLVAKQSVVKFQLKTADLPAGRDETTSFVLVDPLPSGYQFNPEATKAASPGFDVAYDNATNTVTFKATAATLATFNADLTKSVATIYPTVVGQVLNDGATYKNNFSLTVNDAYGIKSNVVRVTTPGKPNDPDNPNNNYIKPTKVNKNENGVVIDGKTVLAGSTNYYELTWDLDQYKNDRSSADTIQQGFYYVDDYPEEALELRQDLVKITDANGNEVTGVSVDNYTSLEAAPQEIRDVLSKAGIRPKGAFQIFRADNPREFYDTYVKTGIDLKIVSPMVVKKQMGQTGGSYEDQAYQIDFGNGYASNIVINNVPKINPKKDVTLTLDPADTNNVDGQTIPLNTVFNYRLIGGIIPANHSEELFEYNFYDDYDQTGDHYTGQYKVFAKVDITLKNGVIIKSGTELTQYTTAEVDTTKGAITIKFKEAFLRSVSIDSAFQAESYIQMKRIAVGTFENTYINTVNGVTYSSNTVKTTTPEDPADPTDPQDPSSPRTSTVIIYKPQSTAYQPSSVQKTLPNTGVTNNAYMPLLGIIGLVTSFSLLGLKAKKD

SEQ ID NO: 22 - DNA, 4865 bp, Streptococcus mutans
atttcagcaaaaattgacaaatcaaatcaattatattacaattttttaacgtatattacaaaaatatatttggaagatttattcagatttggaggatttatgaaagtcaaaaaaacttacggttttcgtaaaagtaaaattagtaaaacactgtgtggtgctgttctaggaacagtagcagcagtctctgtagcaggacaaaaggtttttgccgatgaaacgaccactactagtgatgtagatactaaagtagttggaacacaaactggaaatccagcgaccaatttgccagaggctcaaggaagtgcgagtaagcaagctgaacaaagtcaaaccaagctggagagacaaatggttcataccattgaagtacctaaaactgatcttgatcaagcagcaaaagatgctaagtctgctggtgtcaatgttgtccaagatgccgatgttaataaaggaactgttaaaacagctgaagaagcagtccaaaaagaaactgaaattaaagaagattacacaaaacaagctgaggatattaagaagacaacagatcaatataaatcggatgtagctgctcatgaggcagaagttgctaaaatcaaagctaaaaatcaggcaactaaagaacagtatggaaaagatatggtagctcataaagccgaggttgaacgcattaatgctgcaaatgctgccagtaaaacagcttatgaagctaaattggctcaatatcaagcagatttagcagccgttcaaaaaaccaatgctgccaatcaagcatcctatcaaaaagcccttgctgcttatcaggctgaactgaaacgtgttcaggaagctaatgcagccgccaaagccgcttatgatactgctgtagcagcaaataatgccaaaaatacagaaattgccgctgccaatgaagaaattagaaaacgcaatgcaacggccaaagctgaatatgagactaagttagctcaatatcaagctgaactaaagcgtgttcaggaagctaatgccgcaaacgaagcagactatcaagctaaattgaccgcctatcaaacagagcttgctcgcgttcagaaagccaatgcagatgctaaagcggcctatgaagcagctgtagcagcaaataatgccaaaaatgcggcacttacagctgaaaatactgcaattaagcaacgcaatgagaatgctaaggcgacttatgaagctgcactcaagcaatatgaggctgatttggcagcggtgaaaaaagctaatgccgcaaacgaagcagactatcaagctaaattgaccgcctatcaaacagagctcgctcgcgttcaaaaggccaatgcggatgctaaagcggcctatgaagcagctgtagcagcaaataatgccgcaaatgcagcgctcacagctgaaaatactgcaattaagaagcgcaatgcggatgctaaagctgattacgaagcaaaacttgctaagtatcaagcagatcttgccaaatatcaaaaagatttagcagactatccagttaagttaaaggcatacgaagatgaacaagcttctattaaagctgcactggcagaacttgaaaaacataaaaatgaagacggaaacttaacagaaccatctgctcaaaatttggtctatgatcttgagccaaatgcgaacttatctttgacaacagatgggaagttccttaaggcttctgctgtggatgatgcttttagcaaaagcacttcaaaagcaaaatatgaccaaaaaattcttcaattagatgatctagatatcactaacttagaacaatctaatgatgttgcttcttctatggagctttatggcaattttggtgataaagctggctggtcaacgacagtaagcaataactcacaggttaaatggggatcggtacttttagagcgcggtcaaagcgcaacagctacatacactaacctgcagaattcttattacaatggtaaaaagatttctaaaattgtctacaagtatacagtggaccctaagtccaagtttcaaggtcaaaaggtttggttaggtatttttaccgatccaactttaggtgtttttgcttccgcttatacaggtcaagttgaaaaaaacacttctatttttattaaaaatgaattcactttctatgacgaagatggaaaaccaattaattttgataatgcccttctatcagtagcttctcttaaccgagaaaataattctattgagatggccaaagattatacgggtaaatttgtcaaaatctctggatcatctatcggtgaaaagaatggcatgatttatgctacagatactctcaactttaggcagggtcaaggtggtgctcgttggaccatgtataccagagctagcgaaccgggatctggctgggatagttcagatgcgcctaactcttggtatggtgctggtgctatccgcatgtctggtcctaataacagtgtgactttgggtgctatctcatcaacacttgttgtgcctgctgatcctacaatggcaattgaaaccggcaaaaaaccaaatatttggtattcattaaatggtaaaatccgtgcggttaatcttcctaaagttactaaggaaaaacccacacctccggttaaaccaacagctccaactaaaccaacttatgaaacagaaaagccattaaaaccggcaccagtagctccaaattatgaaaaggagccaacaccaccgacaagaacaccggatcaagcagagccaaagaaacccactccgccgacctatgaaacagaaaagccgttggagccagcacctgttgagccaagctatgaagcagagccaacaccgccgacaaggacaccggatcaggcagagccaaataaacccacaccgccgacctatgaaacagaaaagccgttggagccagcacctgttgagccaagctatgaagcagagccaacgccaccgacaccaacaccagatcaaccagaaccaaacaaacctgttgagccaacttatgaggttattccaacaccgccgactgatcctgtttatcaagatcttccaacacctccatctataccaactgttcatttccattactttaaactagctgttcagccgcaggttaacaaagaaattagaaacaataacgatgttaatattgacagaactttggtggctaaacaatctgttgttaagttccagctgaagacagcagatctccctgctggacgtgatgaaacaacttcctttgtcttggtagatcccctgccatctggttatcaatttaatcctgaagctacaaaagctgccagccctggctttgatgtcgcttatgataatgcaactaatacagtcaccttcaaggcaactgcagcaactttggctacgtttaatgctgatttgactaagtcagtggcaacgatttatccaacagtggtcggacaagttcttaatgatggcgcaacttataagaataatttctcgctcacagtcaatgatgcttatggcattaaatccaatgttgttcgggtgacaactcctggtaaaccaaatgatccagataacccaaataataattacattaagccaactaaggttaataaaaatgaaaatggcgttgttattgatggtaaaacagttcttgccggttcaacgaattattatgagctaacttgggatttggatcaatataaaaacgaccgctcttcagcagataccattcaacaaggattttactatgtagatgattatccagaagaagcgcttgaattgcgtcaggatttagtgaagattacagatgctaatggcaatgaagttactggtgttagtgtggataattatactagtcttgaagcagcccctcaagaaattagagatgttctttctaaggcaggaattagacctaaaggtgctttccaaattttccgtgccgataatccaagagaattttatgatacttatgtcaaaactggaattgatttgaagattgtatcaccaatggttgttaaaaaacaaatgggacaaacaggcgggagttatgaagatcaagcttaccaaattgactttggtaatggttatgcatcaaatatcgttatcaataatgttcctaagattaaccctaagaaagatgtgaccttaacacttgatccggctgatacaaataatgttgatggtcagactattccacttaatacagtctttaattaccgtttgattggtggcattatccctgcaaatcactcagaagaactctttgaatacaatttctatgatgattatgatcaaacaggagatcactatactggtcagtataaagtttttgccaaggttgatatcactcttaaaaacggtgttattatcaagtcaggtactgagttaactcagtatacgacagcggaagttgataccactaaaggtgctatcacaattaagttcaaggaagcctttctgcgttctgtttcaattgattcagccttccaagctgaaagttatatccaaatgaaacgtattgcggttggtacttttgaaaatacctatattaatactgtcaatggggtaacttacagttcaaatacagtgaaaacaactactcctgaggatcctgcagaccctactgatccgcaagatccatcatcaccgcggacttcaactgtaattatctacaaacctcaatcaactgcttatcaaccaagctctgtccaaaaaacgttaccaaatacgggagtaacaaacaatgcttatatgcctttacttggtattattggcttagttactagttttagtttgcttggcttaaaggctaagaaagattgacagcatagatattacattagaattaaaaagtgagatgaagcgataaatcacagattgagcttttatctcattttttgatt

SEQ ID NO: 23 - protein, 330 aa, Neisseria meningitidis
MKTSIRYALLAAALTAATPALADITVYNGQHKEAAQAVADAFTRATGIKVKLNSAKGDQLAGQIKEEGSRSPADVFYSEQIPALATLSAANLLEPLPASTINETRGKGVPVAAKKDWVALSGRSRVVVYDTRKLSEKDLEKSVLNYATPKWKNRIGYAPTSGAFLEQVVAIVKLKGEAAALKWLKALKEYGKPYAKNSVALQAVENGEIDAALINNYYWHAFAREKGVQNVHTRLNFVRHRDPGALVTYSGAVLKSSQNKDEAKKFVAFLAGKEGQRALTAVRAEYPLNPHVVSTFNLEPIAKLEAPQVSATTVSEKEHATRLLEQAGMK

SEQ ID NO: 24 - DNA, 1072 bp, Neisseria meningitidis
atgaaaacatctatccgatacgcactgcttgccgcagccctgaccgccgcaacccccgcgctggcagacattaccgtgtacaacggccaacacaaagaagcagcacaagccgttgcagatgcctttacccgggctaccggcatcaaagtcaaactcaacagtgccaaaggcgaccagcttgccggtcaaatcaaagaagaaggcagccgaagccccgccgacgtattctattccgaacaaatcccggcactcgccaccctttccgcagccaacctcctagagcccctgcccgcctccaccatcaacgaaacacgcggcaaaggcgtgccggttgccgccaaaaaagactgggtggcactgagcggacgttcgcgcgtcgtcgtttacgacacccgcaaactgtctgaaaaagatttggaaaaatccgtcctgaattacgccacgccgaaatggaaaaaccgcatcggttacgcccccacttccggcgcgttcttggaacaggttgtcgccatcgtcaaactgaaaggcgaagcggccgcattgaaatggctcaaagcactgaaagaatacggcaagccttacgctaaaaactccgtcgcccttcaagcggttgaaaacggcgaaatcgatgccgccctcatcaacaactactactggcacgctttcgcgcgtgaaaaaggcgtacaaaatgtccacacccgcctgaatttcgtccgccacagagatcccggcgcactcgttacctattccggcgcagtgttaaaatcctcccaaaacaaggatgaggcgaaaaaattcgtcgccttcctcgccggcaaggaaggacagcgcgccctgaccgccgtccgtgccgaatatcctttgaatccgcacgtggtatccactttcaatttggaacccatcgccaagttggaagcaccccaagtgtccgccaccactgtttccgaaaaagaacacgccacccggctgcttgagcaagccggtatgaaataagccgttttcggattgtcaaacgggtggacatttatactgtccgcccgttttgccgatgaaaaacactatgtctcctaaa

SEQ ID NO: 25 - protein, 556 aa, Streptococcus mutans
MKRKGLRRLLKFFGTVAIILPMFFIALTKAQASDVSSNISSLTVSPTQINDGGKTTVRFEFDEHAQNIKAGDTITVNWQNSGTVRGTGYTKTIKLEVQGKYVGDLVVTQDKAVVTFNDSITGLQNITGWGEFEIEGRNFTDTTTGNTGSFQVTSGGKTAEVTVVKSASGTTGVFYYKTGDMQTDDTNHVRWFLNINNENAYVDSDIRIEDDIQSGQTLDIDSFDITVNGSESYHGQEGINQLAQRYGATISADPASGHISVYIPQGYASLNRFSIMYLTKVDNPDQKTFENNSKAWYKENGKDAVDGKEFNHSVANVNAAGGVDGRTTTTTEKPTTTTEAPTTTETPTTTEAPTTTEAPTTTETPTTTEAPTTEAPTTTEAPTTTEAPTTTEAPTTTEAPTTTEAPTTTEAPTTTEAPTTTEAPTTTEAPTTTEAPTTTEAPTTTEAPTTTEAPTTTEAPTTTGAPTTTEAPTTTEASSETTKAEEKTTEVKEPEKTTTTAPAGKTSNKPNKPSGKQNAGAKGLPSTGEESGTVLSLLGLAAVSMTGLFYYRKHHN

SEQ ID NO: 26 - DNA, 1782 bp, Streptococcus mutans
tacaaagaaatgaaagatgttataatagatttgtaatattcttgttacaagaaaggactaaaaatatgaaaagaaaaggtttacgaagactattaaagttttttggaaccgttgccatcattttgccaatgtttttcatagctttaacgaaagctcaggcaagtgatgtcagcagtaacatttcatcgctgacggtatcaccgactcagattaatgatggcggtaagaccaccgttcgctttgagtttgatgagcatgctcaaaatattaaagcaggcgacaccattactgttaactggcagaattcaggaacagtcagaggaacaggttatacgaaaaccattaagctggaggttcagggcaagtatgttggtgatttggtagttacgcaagacaaagcagttgttactttcaatgacagtattactggcttgcagaatatcaccggctggggtgaatttgaaatcgaaggccggaattttactgacactactaccggaaatactggcagcttccaagttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcacggtcaagaaggtattaatcagcttgcccaaagatatggtgcaactatttcagctgatccggctagtggccatatcagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaatgttaatgccgccggcggtgtggacggaagaacaaccactactacagaaaagccaacaacgacgacagaggctccaacaacaacggaaactccaacgacaacagaggctccaacgacgacagaggctccaacaacaacggaaactccaacgacaacagaggctccaacaacggaagctccaacgacaacagaggctccaacgacaacagaggctccaacaacaacggaagctccaacgacaacagaagctccaacgacaacagaagctccaacaacaacggaagctccaacgacaacagaggctccaacgacaacagaagctccaacaacaacggaagctccaacgacaacagaggctccaacaacaacggaagctccaacaacaacggaagctccaacaacaacagaggctccaacaacgacggaagctccaacgacaacaggggctccaacaacaacggaagctccaacgacgacagaggcatcttcagaaacaacaaaagctgaagaaaagactactgaagttaaggaaccagaaaaaacaacgacaacagctccagcaggtaagacttcaaacaaacctaataagccatcaggcaaacaaaatgctggtgctaagggacttccaagcacaggcgaagaaagcggcactgttttgtcacttctcggtcttgcagctgtctcaatgactggtctattctattaccgtaaacatcataactgatattgattaaaaataggatgaaagaggcagggacaagagtctttgc

SEQ ID NO: 27 - protein, 165 aa, Streptococcus mutans; identical to SEQ ID NO: 5

SEQ ID NO: 28 - DNA, 495 bp, Streptococcus mutans; identical to SEQ ID NO: 6

SEQ ID NO: 29 - protein, 538 aa, Streptococcus mutans
MKRKGLRRLLKFFGTVAIILPMFFIALTKAQASDVSSNISSLTVSPTQINDGGKTTVRFEFDEHAQNIKAGDTITVNWQNSGTVRGTGYTKTIKLEVQGKYVGDLVVTQDKAVVTFNDSITGLQNITGWGEFEIEGRNFTDTTTGNTGSFQVTSGGKTAEVTVVKSASGTTGVFYYKTGDMQTDDTNHVRWFLNINNENAYVDSDIRIEDDIQSGQTLDIDSFDITVNGSESYRGQEGINQLAQRYGATISADPASGHISVYIPQGYASLNRFSIMYLTKVDNPDQKTFENNSKAWYKENGKDAVDGKEFNHSVANVNAAGGVDGRTTTTTEKPTTTTEAPTTTETPTTTEAPTTTESPTTTEAPTTTEAPTTTEAPTTTEAPTTTEAPTTTEAPTTTEAPTTTEAPTTTEAPTTTEAPTTTEAPTTTEAPTTTEAPTTTEAPTTTESPTTTEAPTTTEVSSETTKAEETTTKVKEPEKTTTSVPAGTTSNKPNKPSGKQGAGTKGLPSTGEESGIVLSLLGLATVSVTGLVYRKYHS

SEQ ID NO: 30 - DNA, 1617 bp, Streptococcus mutans
atgaaaagaaaaggtttacgaagactattaaagttttttggaaccgttgccatcattttgccaatgtttttcatagctttaacgaaagctcaggcaagtgatgtcagcagtaacatttcatcgctgacggtatcaccgactcagattaatgatggcggtaagaccaccgttcgctttgagtttgatgagcatgctcaaaatattaaagcaggcgacaccattactgttaactggcagaattcaggaacagtcagaggaacaggttatacgaaaaccattaagctggaggttcagggcaagtatgttggtgatttggtagttacgcaagacaaagcagttgttactttcaatgacagtattactggcttgcagaatatcaccggctggggtgaatttgaaatcgaaggccggaattttactgacactactaccggaaatactggcagcttccaagttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcgcggtcaagaaggtattaatcagcttgcccaaagatatggtgcaactatttcagctgatccggctagtggccatatcagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaatgttaatgccgccggcggtgtggacggaagaacaaccactactacagaaaagccaacaacgacgacagaggctccaacaacaacggaaactccaacgacaacagaggctccaacgacaacagagtctccaacaacaacggaagctccaacgacaacagaagctccaacaacaacggaagctccaacgacaacagaggctccaacgacaacagaggctccaacaacaacggaagctccaacgacaacagaggctccaacaacaacggaagctccaacgacaacagaagctccaacgacaacagaggctccaacaacaacggaagctccaacgacaacagaagctccaacgacaacagaagctccaacaacaacggaagctccaacgacaacagagtctccaacaacaacggaagctccaacaacaacggaagtatcttcagaaacaactaaagctgaagaaacaactactaaagttaaggaaccagaaaaaacaacgacatcagttccagcaggtacaacttcaaacaaacctaataagccatcaggcaaacaaggtgctggtaccaagggacttccaagcacaggcgaagaaagcggtattgttttgtcacttctcggtcttgcaactgtctcagtgactggtctagtttaccgtaaatatcatagctga

SEQ ID NO: 31 - protein, 165 aa, Streptococcus mutans
VTSGGKTAEVTVVKSASGTTGVFYYKTGDMQTDDTNHVRWFLNINNENAYVDSDIRIEDDIQSGQTLDIDSFDITVNGSESYRGQEGINQLAQRYGATISADPASGHISVYIPQGYASLNRFSIMYLTKVDNPDQKTFENNSKAWYKENGKDAVDGKEFNHSVAN

SEQ ID NO: 32 - DNA, 495 bp, Streptococcus mutans
gttaccagcggcggcaagacagctgaggttactgtcgttaaatctgcttcagggactaccggcgttttctactataagactggggatatgcagacagatgacaccaatcatgtgcgctggtttttgaatatcaacaatgagaatgcttatgtagacagtgatattcgtattgaagatgacattcagtctggtcaaactttggatatagacagttttgatattactgtaaatggcagtgagtcttatcgcggtcaagaaggtattaatcagcttgcccaaagatatggtgcaactatttcagctgatccggctagtggccatatcagtgtttatattcctcaaggctatgcttctttgaatcgctttagcatcatgtacttgactaaagttgacaatcctgatcaaaagacgtttgaaaataacagtaaggcttggtataaggaaaacggtaaagatgctgttgatggtaaggaatttaaccattctgtagctaat